(19) United States
(12) Patent Application Publication
(10) Pub. No.: US 2026/0334782 A1
(43) Pub. Date: Oct. 29, 2026

(54) SYSTEM AND METHODS FOR CAPTURING AND PROCESSING IMAGES OF A TARGET BODY PART FOR DIAGNOSTIC PURPOSES

(71) Applicant: InstantDetect Solutions Inc., Sheridan, WY (US)

(72) Inventors: Paul Pacun, New York, NY (US); Michael Ames, Rancho Mission Viejo, CA (US); Steven Martin Cohen, New York, NY (US); Ryan Law, Longmont, CO (US)

(73) Assignee: InstantDetect Solutions Inc., Sheridan, WY (US)

(21) Appl. No.: 19/645,657

(22) Filed: Apr. 13, 2026

Related U.S. Application Data
(60) Provisional application No. 63/790,856, filed on Apr. 18, 2025.

(51) Int. Cl. A61B 3/14; A61B 3/00; A61B 3/11; A61B 3/113; A61B 5/00

(52) U.S. Cl. CPC A61B 3/14; A61B 3/0041; A61B 3/112; A61B 3/113; A61B 5/7267; A61B 5/7415; A61B 5/742

(57) ABSTRACT

A system and method are disclosed for capturing and processing diagnostic images of a target body part using a computing device, thereby transforming the computing device into a calibrated medical imaging device. In at least one embodiment, a three-dimensional audio feedback subsystem guides a user to position the computing device camera normal to the target and capture diagnostic photographs of the target, which are then are evaluated for focus quality, with in-focus portions of the photographs being composited using a focus locus boundary algorithm to produce a fully in-focus composite image. The composite image undergoes illumination and camera color-correction followed by condition-specific color-conditioning to maximize diagnostic discrimination. The conditioned image is analyzed by a machine learning classifier comparing it against labeled condition-positive and condition-negative datasets, yielding a diagnostic result with an associated confidence level, which is encrypted, stored, and transmitted to authorized recipients.

### RELATED APPLICATIONS

This application claims priority and is entitled to the filing date of U.S. provisional application Ser. No. 63/790,856, filed on Apr. 18, 2025. The contents of the aforementioned application are incorporated herein by reference.

### BACKGROUND

The subject of this provisional patent application relates generally to diagnostics, and more particularly to a system and associated methods for capturing and processing images of a target body part for diagnostic purposes.

Applicant hereby incorporates herein by reference any and all patents and published patent applications cited or referred to in this application.

By way of background, capturing and analyzing images of the eye using smartphones can have a multitude of uses. Using smartphones in mobile health (“mHealth”) and smartphones as a medical device (“SaMD”) can be an extremely valuable first line diagnostic tool, especially where early detection can provide greater opportunities for treatment, recovery, or the prevention or mitigation of the severity of disease, an illness, as well as detection of substance impairment, or other detectable health status.

Current smartphone image capturing processes utilize the forward-facing camera, if someone is photographing another person, or the rearward-facing (or “selfie”) camera if someone is photographing themself. Smartphones typically capture higher quality images when the camera is forward-facing and lower quality images when taking a selfie. Further, current smartphone image capturing processes typically focus on the positioning of the eye, getting it as centered as possible within the camera; however this positioning is only part of the solution and a portion of the process. Images might not be clear enough for diagnostic use. Variations in imaging techniques, camera technology, eye angles, and other parameters could lead to inconsistencies in the captured data, multiplying the challenge of comparing and analyzing images across different platforms and settings. Trustworthiness of captured image data is key because the captured image is the start of the diagnostic process and starting with the best possible images will improve reliability. Technological processing advances can improve image trustworthiness when coupled with extended image pre-processing, mathematical transforms of the images obtained, and customization of image base data for the specific pathology or condition being detected.

While it's interesting to note that the general field of photography does not have standardized tests of methodology for determining image clarity, the elements of measurement do exist. These elements include, but are not limited to, image quality, lighting conditions, focal length, camera resolution, field of view, and imaging protocols to name a few. These are key elements to consider when determining clarity.

However, there remains a need for a system capable of capturing and reliably processing images of a target body part in a consistent, repeatable manner for diagnostic purposes. Aspects of the present invention fulfill these needs and provide further related advantages as described in the following summary.

It should be noted that the above background description includes information that may be useful in understanding aspects of the present invention. It is not an admission that any of the information provided herein is prior art or relevant to the presently claimed invention, or that any publication specifically or implicitly referenced is prior art.

### SUMMARY

Aspects of the present invention teach certain benefits in construction and use which give rise to the exemplary advantages described below.

The present invention solves the problems described above by providing a system and associated methods for capturing and processing diagnostic images of a target body part using a computing device such as a smartphone, thereby transforming the computing device into a calibrated medical imaging device. In at least one embodiment, a three-dimensional audio feedback subsystem guides a user to position the smartphone camera normal to the target and at a correct focal length using at least one angular alignment sound proportional to horizontal and vertical displacement, and a focal distance sound proportional to distance from an optimal focal length. One or more diagnostic photographs are evaluated for focus quality, and in-focus portions of multiple photographs are composited using a focus locus boundary algorithm to produce a fully in-focus composite image. The composite image undergoes illumination and camera color-correction followed by condition-specific color-conditioning to maximize diagnostic discrimination. The conditioned image is analyzed by a machine learning classifier comparing it against labeled condition-positive and condition-negative datasets, yielding a diagnostic result with an associated confidence level, which is encrypted, stored, and transmitted to authorized recipients.

As trustworthiness increases, the use of smartphones in mHealth will become a more powerful tool for improving healthcare access, detection, delivery, and outcomes. Creating a standardized and more detailed process for image capture will not only pave the way for more and faster applications for prevention, it can also generate greater diagnostic trustworthiness and opportunities amongst healthcare professionals and end-users.

Using a smartphone as a medical device has unique benefits as well. The entire process of taking photographs and creating diagnostic-worthy images, coupled with pre- and post-image processing, and initiating diagnostic algorithms running on the smartphone not only improves performance because results are instantaneous, but enables the health professional or user to self-test without access to the internet. This is ideal in data dead zones or locations of limited bandwidth, or when time is critical and full duplex access to a host server is not possible.

Also, multiple tests can be performed on the same base image. Once an image has been captured and stored it can be used again in the future to test against other models when available. Several illnesses include but are not limited to covid, malaria, diabetic retinopathy, conjunctivitis, organ disease, and substance impairment to name a few.

Another significant benefit is the low cost, thus encouraging frequent testing. This technology produces no waste or hazardous medical waste or biohazards requiring special handling and disposal since only information is being processed, transmitted, received, and stored.

Other features and advantages of aspects of the present invention will become apparent from the following more detailed description, taken in conjunction with the accompanying drawings, which illustrate, by way of example, the principles of aspects of the invention.

### BRIEF DESCRIPTION OF THE DRAWINGS

The accompanying drawings illustrate aspects of the present invention. In such drawings:

FIG. 1A depicts a typical smartphone used to photograph an image or eye, and the X-Y-Z coordinates are defined, in accordance with at least one embodiment;

FIG. 1B depicts a side view of the smartphone of FIG. 1A, in accordance with at least one embodiment;

FIG. 1C depicts a top view of the smartphone of FIG. 1A, in accordance with at least one embodiment;

FIG. 2 depicts the coordinate system as viewing an eye, in accordance with at least one embodiment;

FIG. 3 depicts the image display area of a smartphone, in accordance with at least one embodiment;

FIG. 4 depicts the top views of three positions of a smartphone imaging an eye and the respective display area corresponding to each of the three respective positions, in accordance with at least one embodiment;

FIG. 5 depicts the side views of three positions of a smartphone imaging an eye and the respective display area corresponding to each of the three respective positions, in accordance with at least one embodiment;

FIG. 6A depicts the display area image of a foreshortened circular target as viewed by the camera when the smartphone is off axis to the right or to the left, in accordance with at least one embodiment;

FIG. 6B depicts the display area image of a target circle as viewed by the camera when the camera is aligned with and directly in front of and perpendicular to the target circle, in accordance with at least one embodiment;

FIG. 7 depicts a diagonally oriented ellipse in the first quadrant of the display area, thus revealing yaw, pitch, ΔXeo, and ΔYeo correction to center the ellipse at the origin of the display area, in accordance with at least one embodiment;

FIG. 8 is a flow diagram of an exemplary method for guiding a user via audio signals to move a phone to take a picture with the pupil centered about the origin, in accordance with at least one embodiment;

FIGS. 9A and 9B depict two photos, each of which has a portion of area in and out of focus, and the in-focus portions of both photos overlap to produce a composite image that is completely in focus as shown in FIGS. 9C and 9D, in accordance with at least one embodiment;

FIGS. 9C and 9D depict composite images of the overlapped in-focus portions of the photos depicted in FIGS. 9A and 9B, in accordance with at least one embodiment;

FIG. 9E depicts three photos, each of which has a portion of area in and out of focus, and the in-focus portions of all three photos overlap to produce a composite image that is completely in focus as shown in FIG. 9D, in accordance with at least one embodiment;

FIG. 9F depicts a single image P1i photo 1 in-focus with no further photos necessary, in accordance with at least one embodiment;

FIG. 10 is a flow diagram of an exemplary method for creating an in-focus image or composite image, in accordance with at least one embodiment, which comprises taking a first photo or frame in a video or image from an array of at least one photo or a multiplicity of photos and determining if the entire photo is in focus, and if only a portion of the photo is in focus defining the area that is in focus, and repeating the process with other photos and combining the in-focus portions of multiple photos to construct a composite image that is completely in focus;

FIG. 11A depicts an exemplary topology, in accordance with at least one embodiment, wherein an in-focus composite image composed of three in-focus sub-images are stitched together at boundaries, and each sub-image has different intensities at each respective boundary;

FIG. 11B depicts a further exemplary topology, in accordance with at least one embodiment, wherein an in-focus composite image composed of three in-focus sub-images are stitched together at boundaries, and each sub-image has different intensities at each respective boundary;

FIG. 11C depicts an exemplary donut topology of an in-focus composite image composed of two in-focus sub-images that are stitched together at a continuous boundary wherein area B is completely enclosed within area A, in accordance with at least one embodiment;

FIG. 12A depicts a closeup of the pixel clusters on each respective side of a generalized tiling boundary, the sum of which produce a single brightness value used to equalize the brightness at an interface boundary, in accordance with at least one embodiment;

FIG. 12B depicts a closeup view of a square pixel cluster comprised of 16 pixels, in accordance with at least one embodiment;

FIG. 12C depicts one possible 16 pixel example of the pixel clusters depicted in FIG. 12B where there are 4 rows and 4 columns for both the A and B sides, in accordance with at least one embodiment;

FIG. 12D depicts a closeup view of a rectangular pixel cluster comprised of 16 pixels, in accordance with at least one embodiment;

FIG. 12E depicts one possible 16 pixel example of the pixel clusters depicted in FIG. 12D where there are 8 rows and 2 columns for both the A and B sides, in accordance with at least one embodiment;

FIG. 12F depicts the full extent of the generalized tiling focus intersection boundary ab, showing the Aab edge of interface boundary in image A and the Bba edge of interface boundary in image B spanning all pixel clusters from the first to the Lth, and identifying the point of equality between the A-side and B-side edge values, and the differential delta values at the first and Lth pixel cluster locations, in accordance with at least one embodiment;

FIG. 12G shows four A image pixels adjoining four B image pixels along the ith row at the Tfbab generalized tiling focus intersection boundary, in accordance with at least one embodiment;

FIG. 12H depicts a typical representation of the values of eight pixels shown in FIG. 12G, in accordance with at least one embodiment;

FIG. 12I depicts the merging process of the two curves depicted in FIG. 12H, in accordance with at least one embodiment;

FIG. 12J shows how a quantified individual pixel value is established from a higher resolution theoretical target value, in accordance with at least one embodiment;

FIG. 13 depicts a closeup of the interface between image B which is completely enclosed with image A in a donut topology as depicted in FIG. 11C, in accordance with at least one embodiment;

FIG. 14A depicts a display screen displaying what the camera sees, and the display shows the zones affecting the sounds produced as a function of the phone camera's position relative to a target, in accordance with at least one embodiment;

FIG. 14B depicts a first sound which is a frequency change as the target or eye moves horizontally across from off the display screen to the left or west across the screen to off the display screen to the right or east, in accordance with at least one embodiment;

FIG. 14C depicts a second sound that is a pulse train which changes its period as a function of the vertical position, and this provides feedback to a user to center a phone's camera to be centered over a target or eye, in accordance with at least one embodiment;

FIG. 14D depicts a third sound that is used to provide guiding distance feedback after the camera is centered over the target or eye;

FIG. 15A depicts a CIE chromaticity chart, which depicts all colors perceivable by the human eye, in accordance with at least one embodiment;

FIG. 15B duplicates the triangle of colors that can be matched by R, B, G, and depicts the application of color correction to a camera's raw image or composite image, in accordance with at least one embodiment;

FIG. 15C depicts the application of color conditioning to increase the sensitivity of machine based discrimination between having and not having a condition of interest, in accordance with at least one embodiment;

FIG. 16A is a flow diagram of an exemplary method for generating three components of positional feedback to optimally position a phone camera and to take pictures of a target or eye, in accordance with at least one embodiment;

FIG. 16B is a flow diagram of an exemplary method for generating two components of positional feedback to optimally position a phone camera and to take pictures of a target or eye, in accordance with at least one embodiment;

FIG. 17 is a flow diagram of an exemplary method for positioning a camera with the aid of audio feedback to take a series of photos as depicted in FIGS. 16A and 16B, determining that at least one photograph or composite photograph is in focus, color correcting, and color conditioning as depicted in FIG. 10, then employing machine learning to compare at least one image to a first set of images possessing the shared characteristics of being representative of a condition and comparing that same image to a second set of images possessing a shared characteristic of not possessing the same condition to determine whether at least one image does or doesn't belong to the set possessing shared characteristics along with an associated confidence level for belonging to that set, in accordance with at least one embodiment; and

FIG. 18 is a flow diagram of an exemplary method for downloading and launching a software application onto a smart device, identification of the smart device parameters and aquation of meta data, the registration process, and training on the use of a smart device to take photographs useful for diagnostic purposes, in accordance with at least one embodiment.

The above described drawing figures illustrate aspects of the invention in at least one of its exemplary embodiments, which are further defined in detail in the following description. Features, elements, and aspects of the invention that are referenced by the same numerals in different figures represent the same, equivalent, or similar features, elements, or aspects, in accordance with one or more embodiments.

### DETAILED DESCRIPTION

Described herein are exemplary embodiments of an imaging system and associated methods for capturing and processing images of a target body part for diagnostic purposes. The imaging system employs end-to-end encryption of all diagnostic images, classification results, and associated personally identifiable information and protected health information (PHI) at every stage of storage and transmission. The encryption architecture is designed to comply with applicable healthcare data protection regulations including but not limited to the Health Insurance Portability and Accountability Act (HIPAA), the European General Data Protection Regulation (GDPR), and equivalent regulations in other jurisdictions. Encryption is applied at the earliest possible point in the data lifecycle, immediately after a diagnostic photograph or composite diagnostic photograph is generated and before any transmission to any external device, server, or cloud resource.

In one embodiment, each diagnostic image is encrypted using a symmetric encryption algorithm providing at least 256 bits of security, including but not limited to AES-256, using a per-image encryption key. The per-image encryption key is itself encrypted using an asymmetric key pair associated with the authorized recipient or recipients of the diagnostic data, such that only authorized recipients possessing the corresponding private key can decrypt the per-image encryption key and subsequently decrypt the diagnostic image. This hybrid encryption scheme provides the efficiency of symmetric encryption for large image payloads combined with the key management flexibility of asymmetric cryptography.

In another embodiment, the imaging system maintains a local encrypted database on the smart device storing diagnostic images, associated metadata including device identifiers, timestamp, GPS coordinates if available and authorized, user identifiers, and classification results with confidence levels. The local database is encrypted at rest using device-level encryption and additionally protected by application-level encryption. A cryptographic hash of each diagnostic image and its associated metadata is computed and stored in the local database at the time of image creation, enabling subsequent verification that the image has not been altered in transmission or storage.

In a further embodiment, the diagnostic data transmission protocol incorporates a mutual authentication handshake in which both the smart device and the receiving server verify each other's identity before any diagnostic data is exchanged. Following mutual authentication, a session key is established using a forward-secrecy protocol such that compromise of long-term key material does not expose past session data. Diagnostic data transmitted within the authenticated session is additionally signed with the user's private key so that the receiving server can verify the provenance of the data and attribute it to the registered user with non-repudiation.

In the following detailed description, the terms cellphone and smartphone are interchangeable, and the definitions herein apply. Specifically, a smartphone or smart device is at least one smart device, a smartphone or what is commonly referred to as a smartphone and can be any technology now known or unknown that can include but is not limited by any specific operating systems, known and unknow including windows, IOS (Apples—iOS, iPadOs, VisionOS, Sonoma), Android, Linux, Unix, typically running on Apple products or iPhone, an Android, any phone that has internet access and can perform WiFi or cellular communication or Bluetooth or nearfield communication or communication of any kind with another device or smart device within range such as wireless earbuds lighting controllers or items that can be incorporated within a servo loop or interactive communication or devices within what is commonly referred to as the “internet of things”. The term “smart device” is intended to include any type of computing or electronic device, now known or later developed, capable of substantially carrying out the functionality described herein-such as server computers, desktop computers, mobile phones, smartphones, laptop computers, tablet computers, personal data assistants, gaming devices, wearable devices, electronic readers, smart displays used in cars, airplanes, etc. As such, the system should not be read as being limited to use with any one particular type of computing or electronic device, even though certain exemplary computing devices may be mentioned or shown herein for illustrative purposes. It should also be noted that, for simplicity purposes, when discussing functionality and the various methods that may be carried out by the system herein, the terms “computing device,” “smart device,” “smartphone” and “user application” are intended to be interchangeable. A smart device can be and incorporate any number of smart devices that cooperatively work together to perform individual or distributed functions. A smart device can also incorporate and communicate with what is commonly referred to as the “cloud”.

It should also be noted that while the system and associated methods are primarily described herein as being used in connection with capturing and processing images of an eye for diagnostic purposes, the system and associated methods should not be so limited. Instead, in further embodiments, the system and associated methods may be utilized in connection with any other body type.

While the detailed description that follows uses the human eye as the primary example of a target body part, it is expressly understood that the imaging system is not limited to ocular imaging. The audio-guided positioning subsystem, focus-locus composite image construction process, color-correction pipeline, and machine learning diagnostic framework described herein are applicable to any target body part that can be photographed by a smartphone camera for diagnostic purposes. The following non-limiting examples illustrate the application of the imaging system to three additional target types.

In a first non-ocular embodiment, the target is a skin lesion or dermatological feature such as a mole, nevus, rash, wound, or pigmented lesion. The user positions the smartphone camera over the skin lesion using the three-dimensional audio feedback subsystem, which guides the camera to be centered over the lesion and at the correct focal distance. The focus-locus composite imaging process constructs a fully in-focus image of the lesion at macro focal length. A condition-specific color-conditioning transform optimized for dermatological features is applied to the composite image prior to machine learning classification. The machine learning classifier evaluates the processed image for malignancy indicators such as asymmetry, border irregularity, color variation, and diameter, enabling smartphone-based screening for melanoma and other skin conditions in settings where dermatological access is limited.

In a second non-ocular embodiment, the target is the interior of the oral cavity, including the throat, tonsils, buccal mucosa, tongue, and gingival tissue. The user employs the smartphone in a rear-facing camera configuration to photograph the oral cavity. A condition-specific color-conditioning transform optimized for oral mucosal tissue can be applied to enhance contrast relevant to streptococcal pharyngitis, oral candidiasis, aphthous ulcers, or early oral carcinoma. The machine learning classifier evaluates the conditioned image against condition-positive and condition-negative datasets specific to the oral target, enabling non-invasive, smartphone-based screening for conditions that currently require in-person clinical examination or throat culture.

In a third non-ocular embodiment, the target is a wound bed or chronic wound, such as a diabetic foot ulcer, pressure injury, or surgical incision site. The audio-guided positioning subsystem ensures that the smartphone is held perpendicular to the wound surface and at a consistent focal distance across serial photographs taken at different time points, enabling quantitative comparison of wound dimensions and tissue composition over time. The focus-locus composite imaging process is particularly valuable for wounds with irregular topography, where different portions of the wound surface are at different focal distances from the camera. The machine learning classifier evaluates the conditioned image to characterize wound tissue composition, detect necrotic tissue, slough, granulation tissue, or infection indicators, and estimate wound area and depth. Serial wound assessments can be transmitted to supervising wound care clinicians in a telehealth workflow.

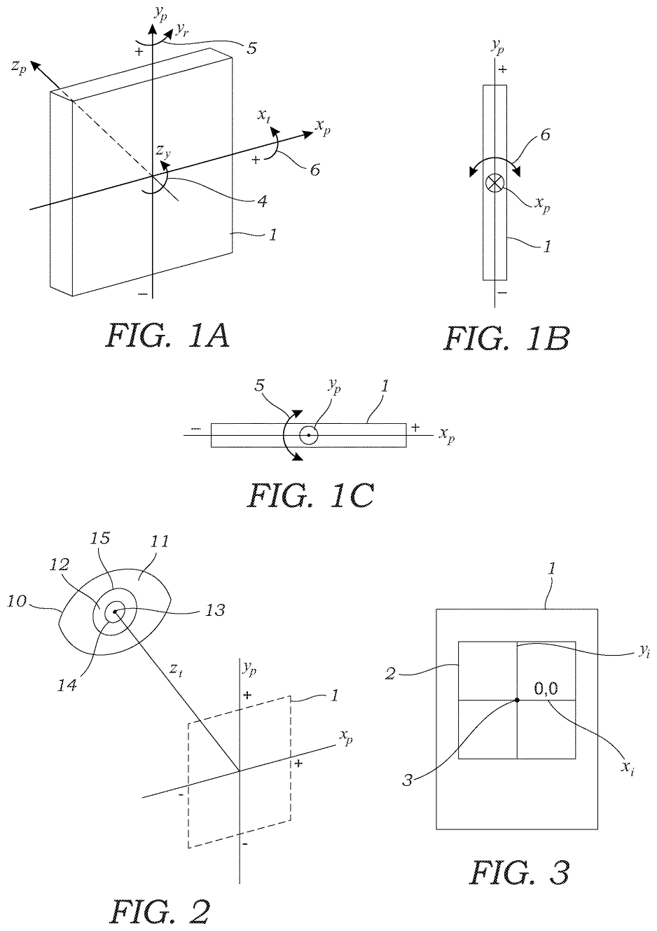

Referring to FIG. 1A, FIG. 1B, FIG. 1C, FIG. 2, and FIG. 3, the following terminology is used to explain how the cellphone 1 must be positioned to get a centered image of the eye 10 on the display area 2. Firstly, the phone axes require naming. When the cellphone 1 is vertically oriented (as one would normally take a picture) the vertical axis is Yp, where the subscript p indicates the phone. Xp is the horizontal axis, and Zt is the axis between the camera and the object being imaged, which is the eye 10, though in the context of the imaging system the object being imaged can be any object. All axes are perpendicular to one another. To establish a convention for rotation, the cellphone 1 can be likened to an airplane moving in the +Yp direction. Rotating cellphone 1 about the Zt axis is referred to as yaw 4. Actually, Zt would be emanating from the camera portion of the cellphone 1, though the figures for convenience show the axis as radiating from the center of the cellphone 1. This location will be different for each camera, but the positioning process will be the same regardless of where the camera happens to be mechanically located within the cellphone 1.

FIG. 1B is a side view of cellphone 1 showing the Yp axis and looking down into the Xp axis (going in and out of the page). The X in the circle indicates looking from the + to − direction down the Xp axis. Note that + indicates increasing values along the Yp axis and − indicates decreasing values along the Yp axis. Rotation of cellphone 1 about the Xp axis is referred to as pitch 6.

FIG. 1C is a top view of cellphone 1 showing the Xp axis (left to right) and looking down into the Yp axis (going in and out of the page). The dot in the circle indicates looking from the − to + direction up the Yp axis. Note that + indicates increasing values along the Xp axis and − indicates decreasing values along the Xp axis. Rotation of cellphone 1 about the Yp axis is referred to as roll 5. So there are three directions of translation of cellphone 1 and three possible rotations about each respective axis, totaling six orthogonal degrees of freedom defining the motion of a cellphone 1.

FIG. 2 depicts the coordinate system as viewing an eye 10. Shown is the vertical axis Yp, the horizontal axis Xp, and Zt, which is the axis to the center of pupil 13. The eye comprises the sclera 11, the iris 12, the iris-sclera boundary 15, which may or may not be round, the pupil 13, and the pupil-iris boundary 14, which is almost always round. It is the 10 pupil-iris boundary 14 and the iris-sclera boundary 15 which the centering process depends upon.

FIG. 3 depicts the image display area 2 of a cellphone 1. Within the display area 2 is the display horizontal axis Xi and display vertical axis Yi, both of which intersect at the origin 3 (0,0). The goal is to align the center of pupil 13 with the origin 3 of display area 2.

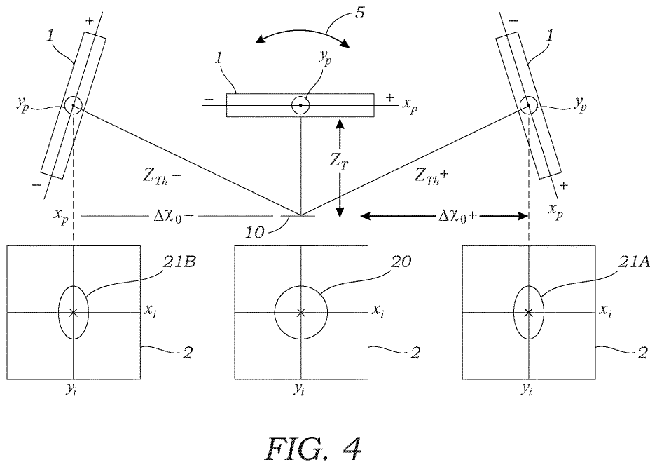

FIG. 4 depicts the top views of three positions of a cellphone 1 imaging an eye 10 and the respective display area 2 corresponding to each of the three respective positions of cellphone 1. Cellphone 1 in the centered position where Zt is perpendicular to both the Xp and Yp axes of the cellphone 1 and entering the center of the pupil 13 of the eye 10. This produces target circle 20, which is either formed by the pupil-iris boundary 14 or the iris-sclera boundary 15. Within the display area 2 is the display horizontal axis Xi and display vertical axis Yi, both of which intersect at the origin 3 (0,0), and this is where the target circle 20 is positioned and centered about. As the cellphone 1 moves horizontally off axis in the + direction a triangle is formed between Zth+, ΔXo+, and Zt. Zth+ is perpendicular to Xp and Yp. As ΔXo+ increases, cellphone 1 is executing roll 5 in the + direction and rotating about the Yp axis to produce the vertical major axis ellipse +21A centered about the origin 3, which is the foreshortening of the target circle 20.

Likewise, as the cellphone 1 moves horizontally off axis in the − direction a triangle is formed between Zth−, ΔXo−, and Zt. Zth− is perpendicular to Xp and Yp. As ΔXo− increases, cellphone 1 is executing roll 5 in the − direction and rotating about the Yp axis to produce the vertical major axis ellipse −21B centered about the origin 3, which is the foreshortening of the target circle 20. Note that for similar geometries vertical major axis ellipse −21B is identical to vertical major axis ellipse +21A, so similar foreshortenings occur when cellphone 1 is translated in either direction from the centered position that produces the target circle 20. This foreshortening will be used to create an audio cue to direct the user to center the cellphone 1.

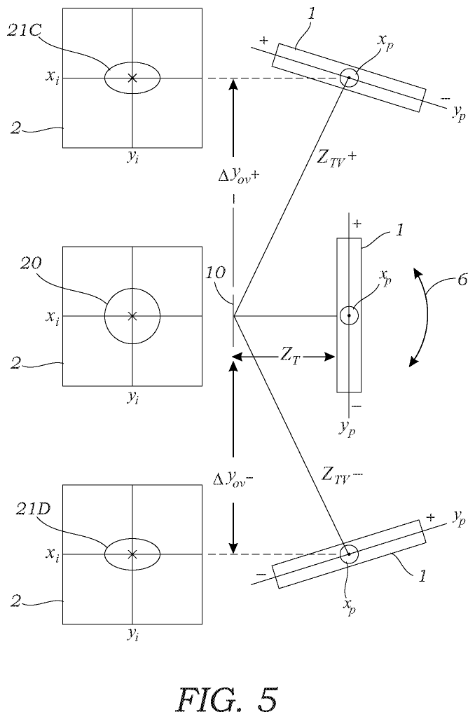

FIG. 5 depicts the side views of three positions of a cellphone 1 imaging an eye 10 and the respective display area 2 corresponding to each of the three respective positions of cellphone 1. Cellphone 1 in the centered position where Zt is perpendicular to both the Xp and Yp axes of the cellphone 1 and entering the center of the pupil 13 of the eye 10. This produces target circle 20, which is either formed by the pupil-iris boundary 14 or the iris-sclera boundary 15 as in FIG. 4. Within the display area 2 is the display horizontal axis Xi and vertical axis Yi, both of which intersect at the origin 3 (0,0), and this is where the target circle 20 is positioned. As the cellphone 1 moves vertically off axis in the + direction a triangle is formed between Ztv+, ΔYo+, and Zt. Ztv+ is perpendicular to Xp and Yp. As ΔYo+ increases, cellphone 1 is executing pitch 6 in the + direction and rotating about the Xp axis to produce the horizontal major axis ellipse +21C centered about the origin 3, which is the foreshortening of the target circle 20.

Likewise, as the cellphone 1 moves vertically off axis in the − direction a triangle is formed between Ztv−, ΔYo−, and Zt. Ztv− is perpendicular to Xp and Yp. As ΔYo− increases, cellphone 1 is executing pitch 6 in the − direction and rotating about the Xp axis to produce the horizontal major axis ellipse −21D centered about the origin 3, which is the foreshortening of the target circle 20. Note that for similar geometries horizontal major axis ellipse −21D is identical to horizontal major axis ellipse +21C, so similar foreshortenings occur when cellphone 1 is translated in either direction from the centered position. This foreshortening will be used to create another audio cue (separate and different than the audio cue above) to direct the user to center the cellphone 1. Two audio cues simultaneously produced will enable the user to center the image and correct for two translation and two rotations simultaneously, and this is one of the key features of the imaging system.

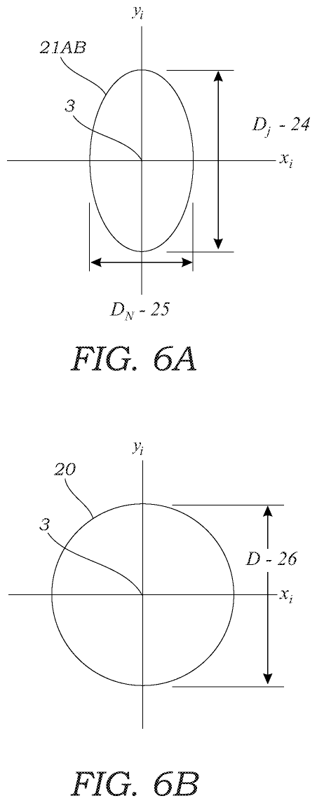

FIG. 6B depicts the display area 2 image of a target circle 20 as viewed by the camera when the camera is aligned with and directly in front of and perpendicular to the target circle 20. The display horizontal axis Xi and display vertical axis Yi. The center of target circle 20 is located at the origin 3, and the target circle diameter 26 is D.

FIG. 6A depicts the display area 2 image of the vertical major axis ellipse 21AB, which is the foreshortened target circle 20 as viewed by the camera when the camera is off axis to the right or to the left and correspondingly rotated in a roll 5 to keep the vertical major axis ellipse 21AB centered about the origin 3. The display horizontal axis Xi and display vertical axis Yi is shown. The vertical major axis ellipse 21AB has an ellipse major diameter 24, or Dj, and an ellipse minor diameter 25, or Dn. In this case the ellipse major diameter 24 is equal to the target circle diameter 26. As the camera moves further off axis the target circle diameter 26 becomes the ellipse minor diameter 25 along the horizontal axis Xi as depicted in FIG. 4. If the cellphone 1 were moved off axis vertically as depicted in FIG. 5 then the major axis and minor axis would switch places, and the major axis would be aligned with the horizontal axis Xi and the minor axis would be aligned with the vertical axis Yi.

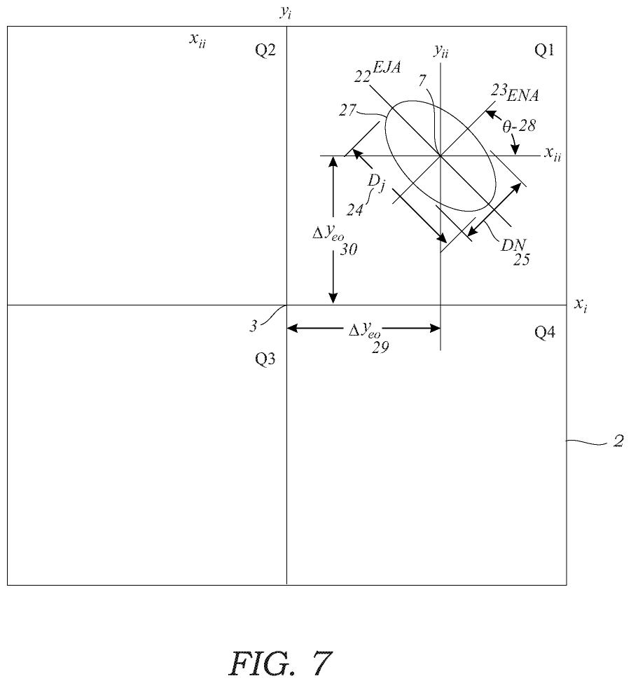

FIG. 7 depicts a diagonally oriented ellipse 27 in the first quadrant Q1 of the display area 2. To produce this the cellphone 1 must roll 5, pitch 6, translate along the Xi axis ΔXeo 29, and translate along the Yi axis ΔYeo 30 to move the cellphone 1 to the diagonally oriented ellipse origin 7 from the origin 3 of the display area 2. So to produce the diagonally oriented ellipse 27 two translations and rotations are necessary, and thus two types of sounds must be simultaneously generated to direct the user to translate and rotate the phone to center position to produce the target circle 20 of FIG. 6B. The diagonally oriented ellipse 27 has ellipse major axis 22 and ellipse minor axis 23 centered about diagonally oriented ellipse origin 7. To determine the angle of the major and minor axes relative to the display horizontal axis Xi and the display vertical axis Yi, a new set of axes are generated at the diagonally oriented ellipse origin 7. Ellipse center horizontal axis Xii is parallel to the display horizontal axis Xi and passes through the diagonally oriented ellipse origin 7 and ellipse center vertical axis Yii is parallel to the display vertical axis Yi and passes through the diagonally oriented ellipse origin 7. Angle θ is the angle between ellipse minor axis 23 and Xii 28. This angle θ is a component used to generate at least one audio tone to guide the user to position cellphone 1 to produce target circle 20.

All ellipses are a subset of the diagonally oriented ellipse 27 whether the ellipse minor axis 23 is on or parallel to the Xi axis or on or parallel to the Yi axis. The diagonally oriented ellipse 27 can be in any quadrant of the display area 2.

The display area 2 of the camera has a horizontal Xi axis and a vertical Yi axis such that the intersection of the Xi axis and the Yi axis is at the origin 3 (or 0, 0), which is the center of the visual display area 2 as seen by the user. When centered, the pupil in the center of the eye is at the origin 3 and the camera is vertically oriented. If the camera is not centered, the pupil 13 at the center of the iris 12 is at position Xii and Yii, where Xii is parallel to the Xi axis of the camera and Yii is parallel to the Yi axis of the camera. The horizontal offset displacement ΔXeo is therefore (Xii-Xi) and the vertical offset ΔYeo displacement is (Yii-Yi).

To produce feedback to the user two different types of audio sounds are simultaneously produced to guide the user to origin 3 the camera's display area 2. A first sound is generated proportional to the absolute value of the displacement ΔXeo 29, or (Xii-Xi) and the roll 5 and a second sound is generated proportional to the absolute value of the displacement ΔYeo 30, or (Yii-Yi) and the pitch 6.

For example only, as |ΔXeo| increases a frequency of an audio tone can increase, so the user will position the camera closer to the Yi axis to cause the frequency to decrease. An increasing frequency can be generated whether the camera is moving too far to the left or too far to the right because if the frequency is increasing the camera is being moved in the wrong direction, so in this manner the audio feedback to minimize the frequency will always guide the user to move the cellphone 1 toward the center the display area 2. Likewise, if a second sound, say a pulsed sound or double beep of one note of one frequency for a short duration and a second note of the same or different frequency of a second duration, is generated proportional to the vertical displacement |ΔYeo|, this will guide the user to position the camera closer to the Xi axis. The double pulse will increase in frequency if the camera is too far above or below the horizontal axis.

These two types of sounds are for example only and there are many sounds that can effectively guide the user to center the camera quickly. After a short learning period the corrective positioning process will become easier.

The perimeter of either the iris 12 or pupil 13 is established, and if out of round and elliptical, the best fit ellipse major axis 22 and best fit ellipse minor axis 23 of the best fit diagonally oriented ellipse 27 is determined. The ellipse minor axis 23 is a measure of the foreshortening of the circular iris 12 or the circular pupil 13, and the angle of the minor axis relative to the ellipse center vertical axis Yii and ellipse center horizontal axis Xii will determine how much angular correction of the cellphone 1 is necessary to produce a circular iris 12 or circular pupil 13. Rotation (roll 5) about the vertical axis Yp of the cellphone 1 produces horizontal motion of the diagonally oriented ellipse 27 and rotation (pitch 6) about the horizontal axis Xp of the cellphone 1 produces vertical motion of the diagonally oriented ellipse 27.

If the cellphone 1 is tilted such that the ellipse minor axis 23 is at an angle θ relative to the Xii and axis and at an angle 90°-θ relative to the Yii axis, then corrective rotation and translation can be affected by the user to produce target circle 20, which can be either the iris 12 or pupil 13 or both and averaged to produce a best fit diagonally oriented ellipse 27.

The goal is to transform the iris's ellipse or the pupil's ellipse into a centered circular image or best fit circular image at the origin 3. It should be understood that when referring to the iris 12 or pupil 13 or any circular image we are referring to the best fit of these shapes.

Rotation and translation of the cellphone 1 are coupled, so if looking only at X motion, cellphone 1 must be both rotated about the vertical axis (roll 5) and translated along the Xp horizontal axis to center the image at the origin 3 as depicted in FIG. 4, and likewise if looking only at Y motion, the phone must be both rotated about the horizontal axis (pitch 6) and translated along the Yp vertical axis to center the image at the origin 3 as depicted in FIG. 5.

The guiding corrective first sound 40 is therefore proportional to a first component of angle θ and to |Δx|, or (1.1) the corrective first sound ∝k1*f1(|Δx|)+k2*f2(θ), where f1 is a first mathematical function and k1 is the weighting factor of the first mathematical function component and f2 is a second mathematical function and k2 is the weighting factor of the second mathematical function component.

The guiding corrective second sound 41 is therefore proportional to a third component of angle θ and to |Δx|, or (1.2) the corrective second sound ∝k3*f3(|Δy|)+k4*f4(θ), where f3 is a third mathematical function and k3 is the weighting factor of the third mathematical function component and f4 is a fourth mathematical function and k4 is the weighting factor of the fourth mathematical function component.

In one embodiment and the simplest form: (2.1) The corrective first sound xx k1*(|Δx|)+k2*sin θ, and (2.2) the corrective second sound ∝k3*(|Δy|). In this embodiment the corrective first sound 40 can be proportional to a first scaling factor k1 multiplying the absolute value |Δx| of the X displacement+a second scaling factor k2 multiplying the sin of the angle θ, and the corrective second sound 41 can be proportional to a third scaling factor k3 multiplying the absolute value |Δy| of the Y displacement.

In a second embodiment and the simplest form: (3.1) The corrective first sound xx k1*(|Δx|), and (3.2) the corrective second sound ox k3*(|Δy|)+k4*sin θ. In this embodiment the corrective first sound 40 can be proportional to a first scaling factor k1 multiplying the absolute value |Δx| of the X displacement, and the corrective second sound 41 can be proportional to a third scaling factor k3 multiplying the absolute value |Δy| of the Y displacement+a fourth scaling factor k4 multiplying the sin of the angle θ.

In a third embodiment: (4.1) The corrective first sound xx k1*(|Δx|)+k2*sin θ, and (4.2) the corrective second sound ∝k3*(|Δy1)+k4*sin θ. In this embodiment the corrective first sound 40 can be proportional to a first scaling factor k1 multiplying the absolute value |Δx| of the X displacement+a second scaling factor k2 multiplying the sin of the angle θ, and the corrective second sound 41 can be proportional to a third scaling factor k3 multiplying the absolute value |Δy| of the Y displacement+a fourth scaling factor k4 multiplying the sin of the angle θ.

In the generalized form of equations 1.1 and 1.2 above the functions f1, f2, f3, and f4 as well as the scaling factors k1, k2, k3, and k4 can be any functions or values that produce the most efficacious corrective feedback sounds. These functions can be linear or non-linear or numerical functions or any combinations thereof.

Corrective sounds can change discretely in a portamento fashion, like running up the keys of a piano from one discrete frequency to the next or corrective sounds can continuously pitch shift up or down in a glissando fashion, like the continuous frequency change of a trombone or slide whistle. The rate of sound change can be proportional to the previous position samples. The frequency change can increase or decrease as a function of the change in position, the rate of change of position, or a combination. For instance, if the camera is taking pictures at 30 frames per second a position is determined every thirtieth of a second. The last sample is the Nth sample, the previous sample before that is the N−1 sample, and the sample before that is the N−2 sample. The present corrective frequency can change as a function of the extrapolation of the curve fit of the last and at least one previous sample. Taking the rate of change of position and angle into account, equations 1.1 and 1.2 become (1.1.1) the corrective first sound 40 ∝k1*f1(|Δx, Δx−1, Δx−2, Δx−3 . . . |)+k2*f2(θ, θ−1, θ−2, θ−3 . . . ), and (1.2.1) the corrective second sound 41 ∝k3*f3 (|Δy, Δy−1, Δy−2, Δy−3 . . . . |)+k4*f4 (θ, θ−1, θ−2, θ−3 . . . ), where Δx and Δy is the most recent position sample, the position sample one time step before that is Δx−1 and Δy−1, the position sample two time steps before is Δx−2 and Δy−2, and so on. Likewise, θ is the most recent angle sample, the angle sample one time step before that is θ−1, the angle sample two time steps before is θ−2, and so on. In this manner the corrective sounds are produced with a time lag of one photograph's delay, or in this case a thirtieth of a second, which would not be too disconcerting a delay. If the photograph rate were 20 frames per second, then there would be a 50 millisecond delay.

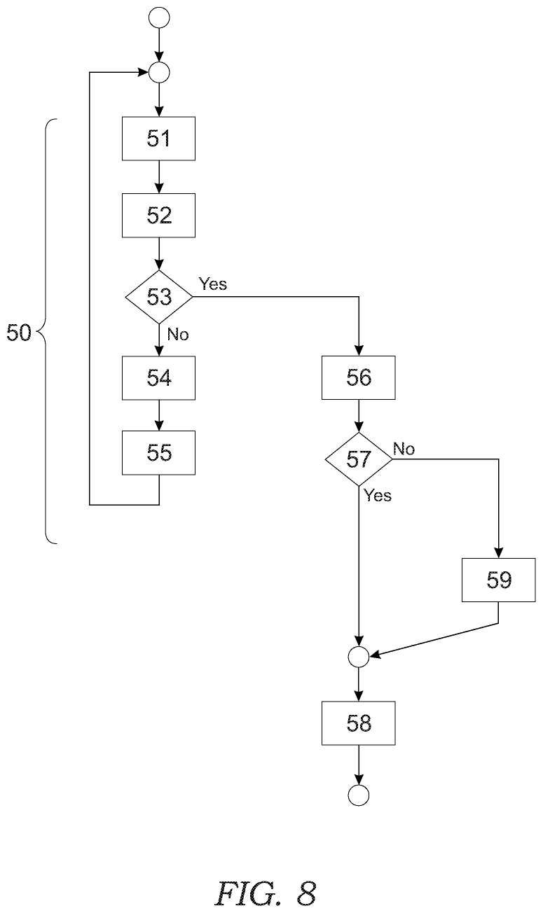

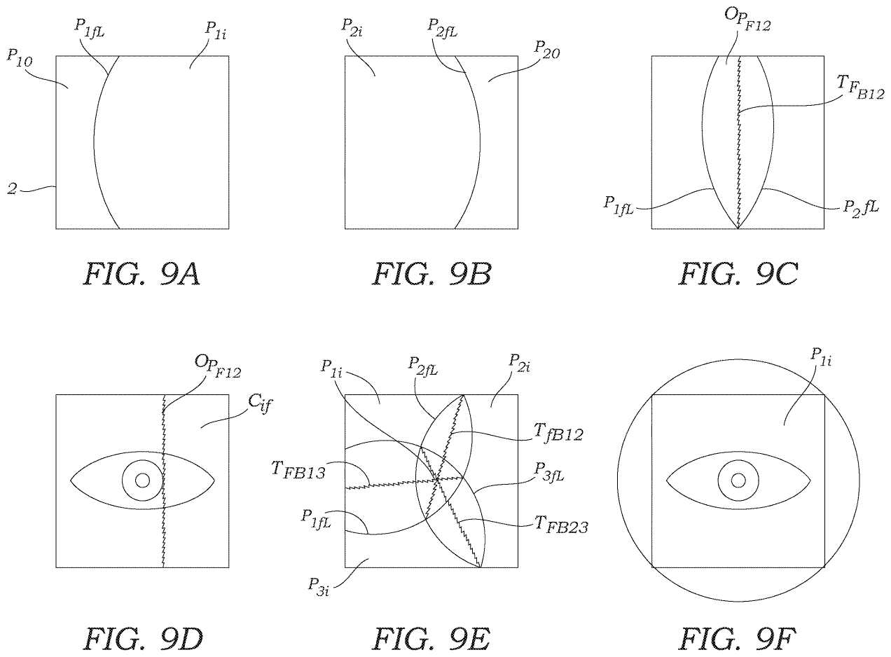

FIG. 8 depicts the image acquisition 50 process of guiding a user via audio signals to move a phone to take at least one photo 51 with the pupil 13 centered about the origin 3. If you're taking a picture of someone else's eye the photographic process is much easier because you're viewing and centering the image as you′d normally take a closeup picture. If you're taking a picture of yourself the process of centering is more complicated in the absence of visual feedback. In the image acquisition 50 mode the camera is either taking pictures continuously or processing the image to provide real time positional and angular feedback. The camera will take at least one photo 51, which is followed by the process to determine position and orientation parameters 52. If ‘no’ to the question Is the image centered at the origin? 53 the phone will generate or alter at least one audio signal 54. In one embodiment this can take the form of generating a tone of a particular frequency as defined above. This causes the user to reposition phone 55, which then leads back to take at least one photo 51 in a closed loop. If ‘yes’ to the question Is the image centered at the origin? 53 the camera will take at least one photo 56, followed by the determination is the photo in focus 57 (i.e., is the image of the target body part within the photo sufficiently sharp, clear, and distinct rather than blurry). If ‘yes’ then store this photo for analysis 58. If ‘no’ create composite picture 59, then store this photo for analysis 58. The process to determine locus of focus 57 as shown in FIG. 9.

FIGS. 9A, 9B, and 9C depict two photos, each of which has a portion of area in and out of focus, and the in-focus portions of both photos overlap to produce a composite image that is completely in focus as shown in FIGS. 9C and 9D. FIG. 9E depicts three photos, each of which has a portion of area in and out of focus, and the in-focus portions of all three photos overlap to produce a composite image that is completely in focus as shown in FIG. 9D.

FIG. 9F shows a single image P1i photo 1 in-focus with no further photos necessary. This is the preferred condition, to photograph the eye and have the entire visual field remain in focus, Failing that, FIG. 9A depicts a first photo, a portion of which is P1i photo 1 in focus, a portion of which is P10 photo 1 out of focus, and the in-focus and out of focus portions of photo 1 meet at P1fl photo 1 focus locus, or the boundary of where the in-focus meets the out of focus. FIG. 9B depicts a second photo, a portion of which is P2i photo 2 in focus, a portion of which is P20 photo 2 out of focus, and the in-focus and out of focus portions of photo 2 meet at P2fl photo 2 focus locus, or the boundary of where the in-focus meets the out of focus. FIG. 9C depicts the overlap of the in-focus portions of photo 1 and photo 2.

Shown is the in-focus portion of photo 1 P1i photo 1 in-focus and the P1fl photo 1 focus locus, or the boundary of where the in focus meets the out of focus. Also shown is the in-focus portion of photo 2 P2i photo 2 in-focus and the P2fl photo 2 focus locus, or the boundary of where the in-focus meets the out of focus. In the area between the P1fl photo 1 focus locus and the P2fl photo 2 focus locus is the Tfb12 tiling focus intersection boundary 12. This can be computed in any number of ways, but for example only this can be the halfway line between P1fl photo 1 focus locus and P2fl photo 2 focus locus, and this guarantees that the composite image is well within the in-focus portion of both photos. This Tfb12 tiling focus intersection boundary 12 is the demarcation between where the P10 photo 1 out of focus portion of photo 1 is truncated and where P20 photo 2 out of focus portion of photo 2 is truncated, thus leaving only the in-focus portions of photo 1 PC1i photo 1 in-focus cropped and photo 2 PC2i photo 2 in-focus cropped to create when logically ORed the entire Cif composite image in-focus filling display area 2 as shown in FIG. 9D.

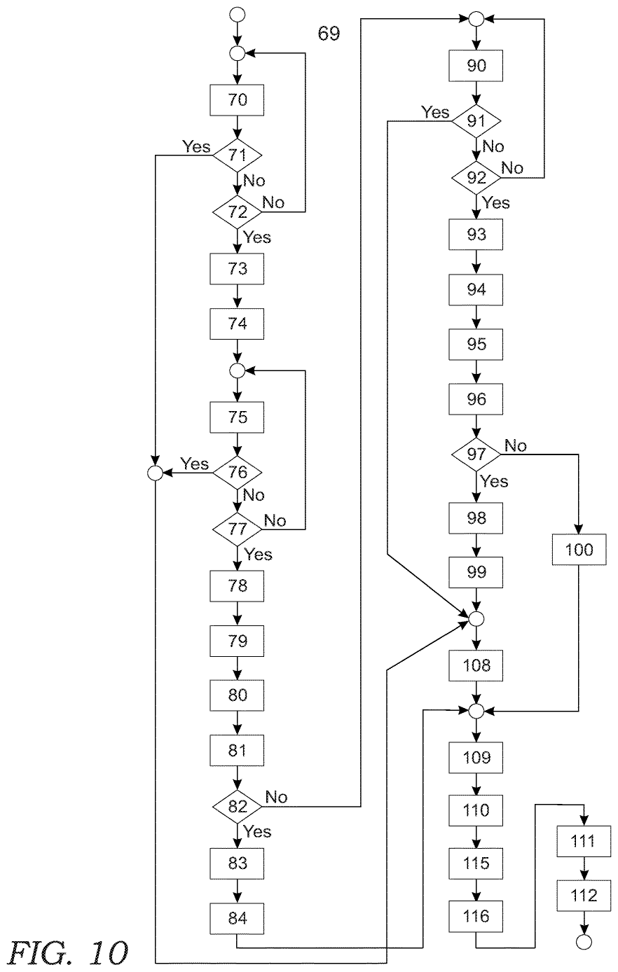

FIG. 9E depicts the in-focus portions of photo 1, photo 2, and photo 3, all of which will be combined to produce Cif composite image in-focus. Shown is the P1i photo 1 in-focus portion of photo 1 and the P1fl photo 1 focus locus, or the boundary of where the in-focus meets the out of focus (not shown) portion of photo 1. Also shown is the P2i photo 2 in-focus portion of photo 2 and the P2fl photo 2 focus locus, or the boundary of where the in-focus meets the out of focus (not shown) portion of photo 2. And also shown is the P3i photo 3 in-focus portion of photo 3 and the P3fl photo 3 focus locus, or the boundary of where the in-focus meets the out of focus (not shown) portion of photo 3. The P1fl photo 1 focus locus and P2fl photo 2 focus locus overlap, and the Tfb12 tiling focus intersection boundary 12 forms the in-focus boundary of the P1i photo 1 in-focus portion of photo 1 and the P2i photo 2 in-focus portion of photo 2. Likewise, the P1fl photo 1 focus locus and P3fl photo 3 focus locus overlap, and the Tfb13 tiling focus intersection boundary 13 forms the in-focus boundary of the P1i photo 1 in-focus portion of photo 1 and the P3i photo 3 in-focus portion of photo 3. And the P2fl photo 2 focus locus and P3fl photo 3 focus locus overlap, and the Tfb23 tiling focus intersection boundary 23 forms the in-focus boundary of the P2i photo 2 in-focus portion of photo 2 and the P3i photo 3 in-focus portion of photo 3. The intersection of Tfb12 tiling focus intersection boundary 12, Tfb13 tiling focus intersection boundary 13, and Tfb23 tiling focus intersection boundary 23 forms CT123 centroid of the tiling focus intersections of Tfb12, Tfb13, and Tfb23. The CT123 centroid of the tiling focus intersections of Tfb12, Tfb13, and Tfb23 and P1i photo 1 in-focus, P2i photo 2 in-focus, and P3i photo 3 in-focus is used to create three cropped and non-overlapping images from PC1i photo 1 in-focus cropped, PC2i photo 2 in-focus cropped, and PC3i photo 3 in-focus cropped. And thus the in-focus portions of three photos can completely fill display area 2. Because these are three separate images taken separately, the lighting at the photo intersections may be different and some blending may be necessary to create a truly seamless image to fill the display area. This process is further elucidated in the flowchart of FIG. 10.

FIG. 10 depicts a flowchart for creating an in-focus image or composite image 69, which comprises taking a first photo or frame in a video or image from an array of at least one photo or a multiplicity of photos and determining if the entire photo is in focus, and if only a portion of the photo is in focus defining the area that is in focus, and repeating the process with other photos and combining the in-focus portions of multiple photos to construct a composite image or photograph that is completely in focus.

In the following and this application a photo means a photograph or video frame or image already stored. It should be noted that the following process is not limited to photos for medical diagnostics of a pathological conditions or impairment due to substances. This process can be applied to photos possessing any visual subject matter.

The first step after positioning the phone is to take or examine photo 1 70. Then the decision must be made: is this photo completely in focus 71? If the photo is completely in focus, then scale to target iris OD 108 and perform a master crop 109. The master crop 109 separates the portion of the in-focus image, photo, or composite photo that is useful for diagnostic purposes from the unnecessary portion. This also reduces the stored data and required bandwidth to upload, download, encrypt, decrypt, and process. The master crop 109 can be a rectangular crop, circular, ellipse, or any bounding shape that normalizes and facilitates diagnostic speed and efficacy. The cropped in-focus photo is now a spatial image, and the next step is to save spatial image 110, which can be a left eye, a right eye, or some other body part. In the context of this patent a spatial image is an image comprising an array of pixels arranged in horizontal rows and vertical columns as pictures are typically stored, and the pixels can possess intensity values of a particular resolution and number of gray scales ranging from 0 to some maximum, which is typically some multiple of 2 raised to the power of N number of bits. The pixels can also comprise red, blue, and green values of particular resolutions, and the weighting of red, blue, and green determines the perceived color of the pixel or cluster of pixels in the image display. As the colors resolvable can only reside within the triangle bounded by the red, blue, and green frequencies located on the CIE color chart, other colors can be used to increase the color space resolvable in future cameras, so this applies to any photographic technology now known or unknown without departing from the spirit of the imaging system.

Supplemental information can be derived from the spatial image via at least one mathematical area transform including but not limited to an FFT, DFT, Laplace transform, wavelet transform or other transform now known or unknown that can be applied to the spatial image or a portion of the spatial image. This step in FIG. 10 is to create at least one transformed version of saved spatial image 111, and then to store at least one transformed image of spatial image 112.

If after take or examine photo 1 70 the photo is not completely in focus 71, then the system must determine if photo 1 is partially in focus 72. If there is no portion of the photo in focus then photo 1 is discarded and another photo 1 is taken and the process repeated. If a portion of the image is in focus then the photo must scale photo 1 to target iris OD 73. Then the system must determine P10, or what portion of photo 1 is out of focus and P1i, or what portion of photo 1 is in focus. Then P1fl, or the in-focus locus will be determined, which is the photo 1 focus locus boundary of where the in-focus portion meets the out of focus portion. This will enable other in-focus portions of photos to be overlaid to produce a composite photo that is completely in focus.

Next the system takes or examines photo 2 75 and determine if it is completely in focus 76. If photo 2 is completely in focus, then scale to target iris OD 108 and perform a master crop 109. Since photo 1 is no longer necessary it is discarded. The same steps as previously performed are now initiated for this spatial image, namely save spatial image 110, create at least one transformed version of saved spatial image 111, then store at least one transformed image of spatial image 112. If photo 2 is not completely in-focus then the system determines whether any portion of photo 2 is partially in focus 77. If no portion of photo 2 is partially in focus the system takes another photo 2 and repeat the above steps. If a portion of photo 2 is partially in-focus then scale photo 2 to target iris OD or feature match 78. Then determine P2i and P2fl photo 2 focus locus 79. This entails determining P20, or what portion of photo 2 is out of focus and P2i, or what portion of photo 2 is in-focus. P2fl, or the in-focus locus will be determined, which is the photo 2 focus locus boundary of where the in-focus portion meets the out of focus portion. This will enable the in-focus portion of photo 1 or other in-focus portions of photos to be overlaid to produce a composite photo that is completely in-focus.

Knowing P1fl and P2fl the system can determine Tfb12 tiling focus intersection boundary 12 from P1fl and P2fl 80 as shown in FIGS. 9C and 9E. Now create PC1i photo 1 in-focus cropped and PC2i photo 2 in-focus cropped 81, and the test must be performed: does PC1i A PC2i cover the full display area? 82. If yes, the system can create Cif composite image in-focus 83 and clean Cif composite image in-focus at tiling interface boundary 84. Then, as before, the system performs a master crop 109, save spatial image 110, create at least one transformed version of saved spatial image 111, and store at least one transformed image of spatial image 112. If however PC1i A PC2i does not cover the full display area, then the system must take or examine photo 3 90 and keep sculpting a composite in-focus image that can produce the most reliable diagnostic results.

Take or examine photo 3 90 and determine if photo 3 is completely in focus 91. If photo 3 is completely in focus, then scale to target iris OD 108 and perform a master crop 109. Since photo 1 and photo 2 are no longer necessary they are both discarded. The same steps as previously performed are now initiated for this spatial image, namely save spatial image 110, create at least one transformed version of saved spatial image 111, then store at least one transformed image of spatial image 112. If photo 3 is not completely in-focus then the system determines whether any portion of photo 3 is partially in focus 92. If no portion of photo 3 is partially in focus the system takes another photo 3 and repeat the above steps. If a portion of photo 3 is partially in-focus then scale photo 3 to target iris OD or feature match 93. Then determine P3i and P3fl photo 3 focus locus 94. This entails determining P30, or what portion of photo 3 is out of focus and P3i, or what portion of photo 3 is in-focus. P3fl, or the in-focus locus will be determined, which is the photo 3 focus locus boundary of where the in-focus portion meets the out of focus portion. This will enable the in-focus portion of photo 1 and photo 2 or other in-focus portions of photos to be overlaid to produce a composite photo that is completely in-focus.

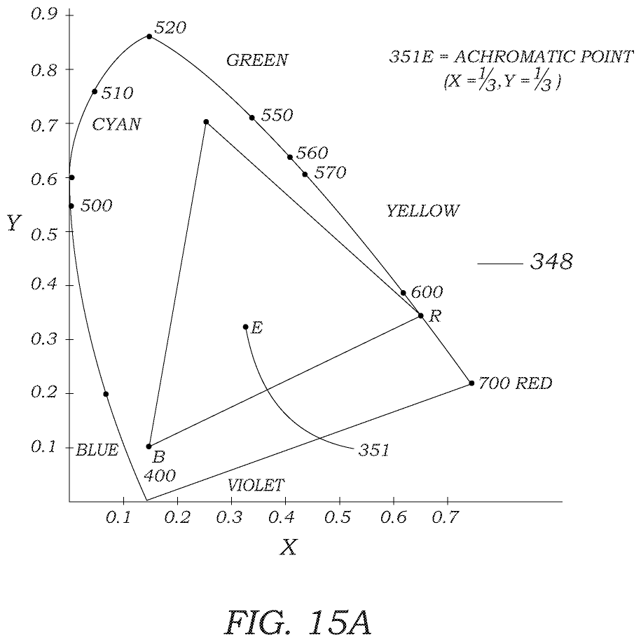

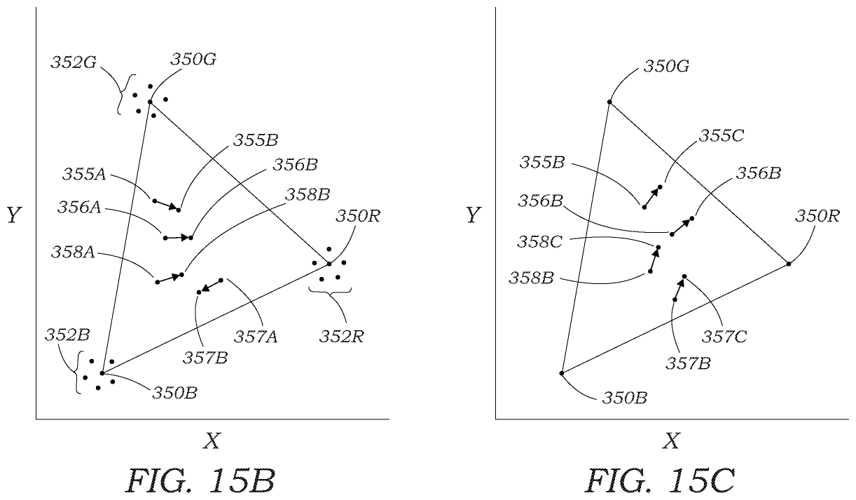

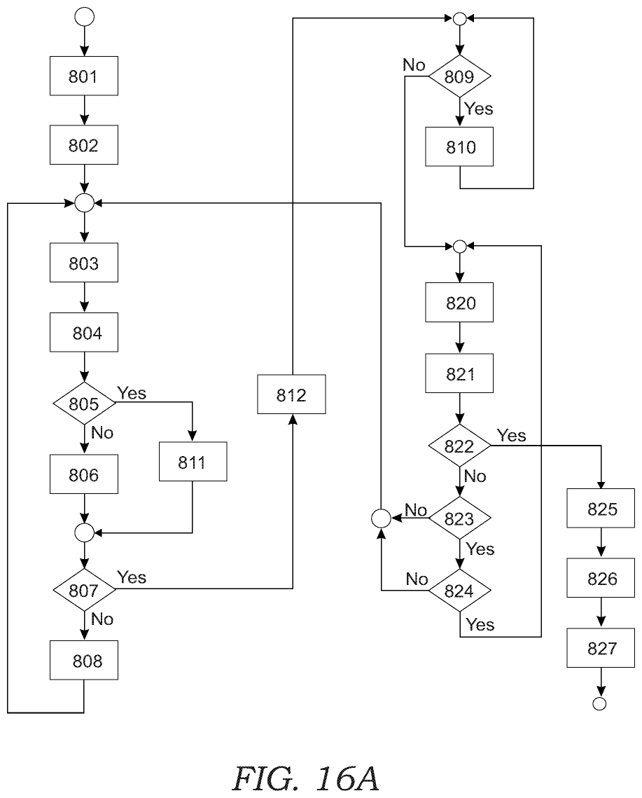

Knowing P1fl, P2fl, and P3fl the system can determine Tfb13 tiling focus intersection boundary 13 from P1fl and P3fl and determine Tfb23 tiling focus intersection boundary 23 from P2fl and P3fl 95 as shown in FIG. 9E. Now create PC3i photo 3 in-focus cropped 96, and in conjunction with PC1i photo 1 and in-focus cropped and PC2i photo 2 in-focus cropped, the test must be performed: does PC1i A PC2i A PC3i cover the full display area? 97. If yes, the system can create Cif composite image in-focus 98 and clean Cif composite image in-focus at tiling interface boundaries 99. Then, as before, the system performs a master crop 109, save spatial image 110, apply a color correction transform 115, which is further elucidated in FIG. 15, apply a color conditioning transform 116, which is further elucidated in FIG. 15, create at least one transformed version of saved spatial image 111, and store at least one transformed image of spatial image 112. If however PC1i A PC2i A PC3i does not cover the full display area, then the system must take yet another photo and repeat generalized steps 90 through 99 incrementing index each pass as many times as necessary to create a clean Cif composite image in-focus 100. Then, as before, the final steps are to perform a master crop 109, save spatial image 110, apply a color correction transform 115, apply a color conditioning transform 116, create at least one transformed version of saved spatial image 111, and store at least one transformed image of spatial image 112. The color correction transform 115 when applied to at least one diagnostic photograph or at least one composite diagnostic photograph can also be referred to as the process of illumination and camera color-correction. The color conditioning transform 116 when applied to at least one diagnostic photograph or at least one composite diagnostic photograph can also be referred to as the process of condition-specific color-correction.

Reference is made to cropping the in-focus and out of focus portions of a photo. One way to crop can be masking the unwanted portions of an image by multiplying the wanted sections or pixels by 1 and the unwanted sections or pixels by 0.

When one photo is tiled next to another photo to maintain focus of the composite image, there may be intensity and color discontinuities. If in-focus image 1 abuts in-focus image 2 along a boundary, the adjoining pixels should be of equal brightness and equal RBG values, but different photos may have different color and intensity values due to the quantization process and variable lighting conditions for the two separate photos even when taken with the same camera. There could therefore be a step variation or delta intensity at the zone of transition from one cropped image to the next. Both images can therefore be blended so a smooth pixel transition can be made. Computationally this might entail gradually altering the pixel values of both pictures, gradually raising the numerical values of bordering pixels of a first photo approaching the transition line while gradually lowering the numerical values of bordering pixels of a second photo approaching the transition line from the opposite direction.

For instance and for demonstrative purposes only, if the following transition occurs between a first photo and a second photo where the pixel values should be the same because the same features are being photographed in both images:
first photo second photo
. . . 50, 50, 50, 50, 50, 50, 50, 50, 61, 61, 61, 61, 61, 61, 61, 61 . . .
   and the value of the transition is to be smoothed, the sequence can be altered as follows:
. . . 50, 50, 50, 51, 52, 53, 54, 55, 56, 57, 58, 59, 60, 61, 61, 61 . . .
   and this smooths the transition over a five-pixel range into the first photo from the transition and five-pixel range into the second photo from the transition. The number of pixels into each range can be preset or can be adaptive, and best results for diagnostic purposes can be experimentally determined.

Another method is to define one photo as the master and all adjacent pictures are completely altered so that all pixels are proportionally raised or lowered in intensity value or RBG levels to create a smooth transition between the master photo and all subordinate photos. This can create a singularity problem if there are three photos, A, B, and C, and A is determined to be the master. In this case photo B is altered to match photo A at the AB boundary, and photo C is altered to match photo A at the AC boundary, but the photo B to photo C boundary may then have a discontinuity, in which case an iterative process can be initiated where all three are altered such that all boundaries have 0 delta intensity and color discontinuity.

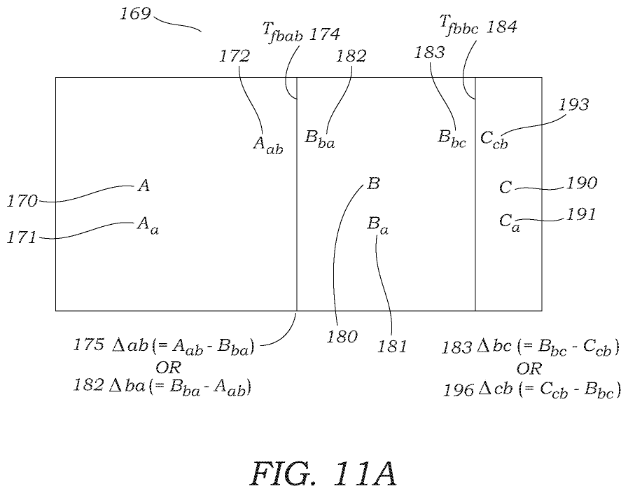

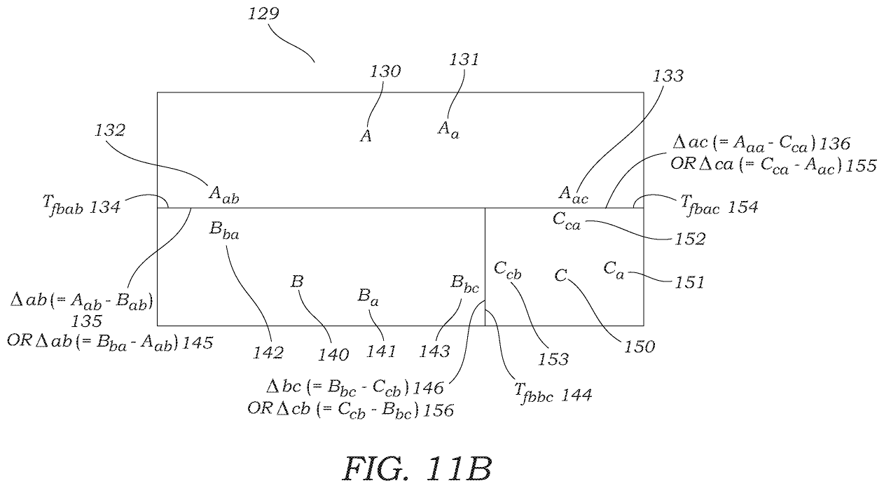

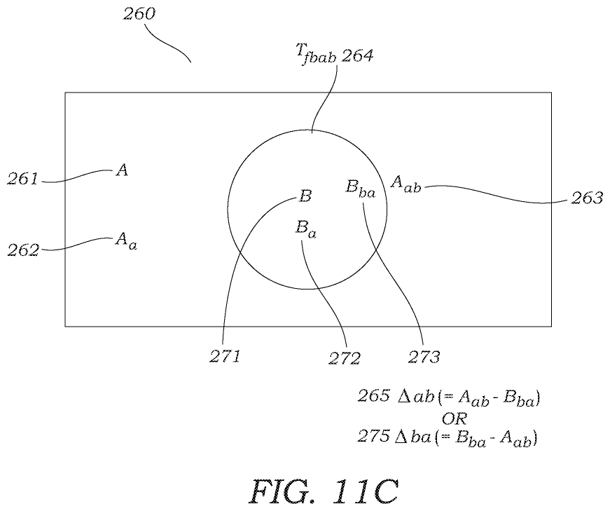

FIG. 11A depicts a topology wherein an in-focus composite image 169 composed of three in-focus sub-images are stitched together at boundaries, and each sub-image has different intensities or color values at each respective boundary. In this topology image A 170 abuts with image B 180 at Tfbab tiling focus intersection boundary ab 174 and image B 180 abuts with image C 190 at Tfbbc tiling focus intersection boundary bc 184, but no portion of image A 170 abuts with image C 190.

Aab value of A at the ab border 172 is the average intensity value of the pixels within image A 170 and can be composed of a multiplicity of pixels of sufficient number as to yield a reliable average intensity. This can also apply to the color value which averages a number of red pixel values, a number of blue pixel values, and a number of green pixel values to produce an average RBG value representing the color.

Bba value of B at the ba border 182 is the average intensity value of the pixels within image B 180 and can be composed of a multiplicity of pixels of sufficient number as to yield a reliable average intensity. As with image A 170, this also applies to color. For the following discussion and for the sake of brevity only intensity will be referred to in the border blending process but it is understood that this equally applies to color as well. Due to the fact that at the Tfbab tiling focus intersection boundary ab 174 the Aab value of A at the ab border 172 may not be equal to Bba value of B at the ba border 182 because they are two separate photographs being mated together. Δab average pixel value difference between Aab and Bba 175 will be equal in magnitude to Δba average pixel value difference between Bba and Aab 185 but opposite in sign.

Similarly, Ccb value of C at the cb border 193 is a numerical average of a multiplicity of representative pixels. Within image B 180, Bbc value of B at the bc border 183 is a numerical average of a multiplicity of representative pixels. As in Tfbab tiling focus intersection boundary ab 174 above, Ccb value of C at the cb border 193 may not be equal to Bbc value of B at the bc border 183 because they are two separate photographs being mated together. Δbc average pixel value difference between Bbc and Ccb 186 will be equal in magnitude to Δcb average pixel value difference between Ccb and Bbc 196 but opposite in sign.

The following example demonstrates how image A 170, image B 180, and image C 190 are adjusted in intensity so that there is no intensity step between the mating boundaries. For the following discussion Aa area of A 171 has an area of 3 units of area, Ba area of B 181 has an area of 2 units of area, and Ca area of C 191 has an area of 1 unit of area, so the total area of the in-focus composite image 169 is Aa+Ba+Ca, or 3+2+1, or 6 total units of area.

For illustrating the intensity boundary equalization of this topology the following values are assigned for illustration only:
Aab value of A at the ab border 172=155
Bba value of B at the ba border 182=145
Bbc value of B at the bc border 183=75
Ccb value of C at the cb border 193=90

With the above values Δab average pixel value difference between Aab and Bba 175 would be 155-145, or 10 intensity units, and Aab would therefore have to decrease in value and Bba would have to increase in value so they can meet somewhere in the middle to eliminate the step in intensity value. And Δbc average pixel value difference between Bbc and Ccb 186 would be 75-90, or −15 intensity units and Bbc would have to increase in value and Ccb would have to decrease in value so they can meet somewhere in the middle to eliminate the step in intensity value.

For the first calculation in the border equalization process, the Aa area of image A 171 has an area of 3 units of area and the Ba area of image B 181 has an area of 2 area units, or a combined total area of Aa+Ba, or 3+2, which equals 5 units of area.

Image A 170 therefore comprises ⅗ or 60% of the combined A-B area, and image B 180 comprises ⅖ or 40% of the A-B combined area of 5 units. Stated generally, A's fraction of the area is Aa/(Aa+Ba) and B's fraction of area is Ba/(Aa+Ba). Because image A 170 has a larger area it will be altered by B's proportion of the combined area, or 40%, or 0.4×10=4 intensity units, and 155−4=151. Because image B 180 has the smaller area it will be altered and raised by A's proportion of the combined area, or 60%, or 0.6×10=6 intensity units, and 145+6=151, so 151 becomes the new intensity value of both Aab and Bba. Because the value of Aab lowered in value, all the pixels within image A 170 also lower, and because the value of Bba has raised in value, all the pixels within image B 180 also raises, and Bbc will also raise by some level of intensity, though not necessarily 4 intensity units as per the color mapping function. For illustrative purposes it will be assumed that the value of Bbc raises by 4 intensity units.

Before any operations, the starting conditions with subscript i=0 are Aab0, Bba0, Bbc0, and Ccb0, and |Δab0|=|Δba0|, and |Δbc0|=|Δcb0|.

So using the above numbers, the starting conditions are:
Aab0=155
Bba0=145
Bbc0=75
Ccb0=90
   |Δab0|=|Δba0|=10
   |Δbc0|=|Δcb0|=15
   After the first operation i=1:
Aab1=151
Bba1=151
Bbc1=75+4=79
Ccb1=Ccb0=90 (unchanged)
   |Δab1|=|Δba1|=0
   |Δbc1|=|Δcb1|=|79-90|=11, the new Δ as a result of the first operation.

For the second operation in the border equalization process, the total area of image A 170, image B 180, and image C is 3+2+1=6 units of area. Aa area of image A 171 has an area of 3 units of area and the Ba area of image B 181 has an area of 2 area units, or a combined total area of Aa+Ba, or 3+2, which equals 5 units of area. Ca area of C 191 has an area of 1 unit of area. The total area of image A 170, image B 180, and image C is 3+2+1=6 units of area. Image A 170+image B therefore comprises ⅚ or 83.33% of the combined A-B-C area, and image C 190 comprises ⅙ or 16.66% of the A-B-C combined area of 6 units. Stated generally, A and B's fraction of the area is (Aa+Ba)/(Aa+Ba+Ca) and C's fraction of area is Ca/(Aa+Ba+Ca). As above, |Δbc1|=|Δcb1|=|79-90|=11, or the Δ as a result of the first operation. A+B's intensity will be altered by C's percentage of the total area and C will be altered by A+B's percentage of the total area, or stated generally:
Bbc2=Bbc1+|Δbc1|×Ca/(Aa+Ba+Ca), or Bbc2=79+11×0.166=80.833, and
Ccb2=Ccb1−|Δcb1|×(Aa+Ba)/(Aa+Ba+Ca), or Ccb2=90−11×0.833=80.833, and thus the combined area of A and B has raised in intensity in the direction of C and C has lowered in intensity in the direction of the combined area of A and B. As a result of this second operation Aab2=Bba2=Aab1+|Δbc1|×Ca/(Aa+Ba+Ca)=151+1.833=152.833, and consequently all pixels within A and B have raised in intensity and all pixels within C have lowered in intensity.

So for the final in-focus composite image 169, Aab2=Bba2, Bbc2=Ccb2, and there is no intensity step at either border.

It should be noted that an intensity change at one location in an image may not produce the same numerical intensity at another location.

There are a few color standards and methods for defining color used in both cameras and image display systems.

The CIE color space was defined by the International Commission on Illumination in 1931. This defined three primary colors of stimulation: red with a wavelength of 700.0 nanometers (nm), green with a wavelength of 546.1 nm, and blue with a wavelength of 435.8 nm. The CIE color space is a two-dimensional representation of color in the first quadrant of a graph, with an X horizontal component ranging from the 0 origin to 0.8 on the right, and a Y vertical component ranging from the 0 origin to 0.9 at the top. The graph represents wavelengths of visible light around the perimeter of the graph, with red on the right, blue on the lower left, and red at the top. Viewable colors can be produced by mixing some combination of red, blue, and green sources of light. The red, blue, and green form the vertices of a triangle, and only colors within the triangle can be produced. Contained within this triangle is what is known as CIE white, which is a combination of different percentages of red, blue, and green.

Digital cameras can employ the YCbCr system, where Y represents the luminosity, or luma, which is brightness; Cb represents the chroma blue component or (blue—luma); and Cr represents the chroma red component or (red—luma). This is represented in a two dimensional graph with Cb on the horizontal axis with the scale going from −1 on the left to 0 at the center to +1 on the right, and Cr on the vertical axis with −1 at the bottom to 0 at the center to +1 at the top. In this YCbCr graphic representation, blue is to the right, green is to the lower left, and red to the upper right.

YUV is another color format wherein the palette of colors displays on a two dimensional map. This is represented in a two dimensional graph with U on the horizontal axis with the scale going from −0.5 on the left to 0 at the center to +0.5 on the right, and V on the vertical axis with −0.5 at the bottom to 0 at the center to +0.5 at the top. In this graphic representation purple is in the first quadrant to the upper right, light blue is in the second quadrant to the lower right, green is in the third quadrant to the lower left, and red is in the fourth quadrant to the upper left.

For the purposes of diagnostics any format of color quantization can be used, though some may be more efficacious than others for discriminating between a condition and non-condition.

If an image is composed of 8 bits of red, 8 bits of blue, and 8 bits of green, there are 24 bits of color information per pixel where a pixel of the camera actually comprises 3 pixels, or one for each color. If an entire picture were one solid color where
R=100
G=110
B=120
   could you change manually or by automatic globally applied process it to:
R=150 (Δ=+50)
G=165 (Δ=+55)
B=183 (Δ=+63)
   and have that new setting become the new color of the mono color image? And how is this done? Can you globally change the pixel values according to a formula? For instance, if a color value were 128 (half scale), and you shifted it to 138 (Δ+10), in some other part of the picture were the value 231, the correct delta would not be 10 (or 241), but only 4 because it was closer to full scale saturation. So there would be a transform for R, G, and B where
Prbg1=old value, and
Prbg2=new value
Prbg2=Prbg1+/−(Δrbg)×F(Prbg1),
   where F(Prbg) is a color mapping function applied to ALL the pixel values in the entire image.

In calculating any new color value derived from a A deviation from an old color value, the color mapping function must also include a test to prevent the lowest pixel intensity from being less than 0 and the maximum pixel intensity from being greater than the maximum value, which for the above intensity quantization of 8 bits would be 255. Stated more rigorously,
if Pr1−Δr or Pb1−Δb or Pg1−Δg<0 then Pr2 or Pb2 or Pg2=0, and
if Pr1+Δr or Pb1+Δb or Pg1+Δg>255 then Pr2 or Pb2 or Pg2=255

As future cameras improve the color quantization will resolve beyond 8 bit resolution, thus increasing both resolution, signal to noise ratio, and diagnostic discrimination.

FIG. 11B depicts an in-focus composite image 129 composed of three in-focus sub-images, image A 130 with Aa area of A 131, image B 140 with Ba area of B 141, and image C 150 with Ca area of C 151 that are stitched together at boundaries. Image A 130 borders image B 140 at Tfbab tiling focus intersection boundary ab 134. Image A 130 borders image C 150 at Tfbac tiling focus intersection boundary ac 154. Image B 140 borders image C 150 at Tfbbc tiling focus intersection boundary bc 144. Aab value of A at the ab border 132 is the average intensity value of the pixels within image A 130 and can be composed of a multiplicity of pixels of sufficient number as to yield a reliable average intensity. This can also apply to the color value which averages a number of red pixel values, a number of blue pixel values, and a number of green pixel values to produce an average RBG value representing the color.

Bba value of B at the ba border 142 is the average intensity value of the pixels within image B 140 and can be composed of a multiplicity of pixels of sufficient number as to yield a reliable average intensity. As with area A 130, this also applies to color. For the following discussion and for the sake of brevity only intensity will be referred to in the border blending process but it is understood that this equally applies to color as well. Due to the fact that at Tfbab tiling focus intersection boundary ab 134 Aab value of A at the ab border 132 may not be equal to Bba value of B at the ba border 142 because they are two separate photographs being mated together. Aab average pixel value difference between Aab and Bba 135 will be equal in magnitude to Aba average pixel value difference between Bba and Aab 145 but opposite in sign.

Similarly, at Tfbac tiling focus intersection boundary ac 154 within image A 130, Aac value of A at the ac border 133 is a numerical average of a multiplicity of representative pixels. Within image C 150, Cca value of C at the ca border 152 is a numerical average of a multiplicity of representative pixels. As in Tfbab tiling focus intersection boundary ab 134 above, Aac value of A at the ac border 133 may not be equal to Cca value of C at the ca border 152 because they are two separate photographs being mated together. Δac average pixel value difference between Aac and Cca 136 will be equal in magnitude to Δca average pixel value difference between Cca and Aac 155 but opposite in sign.

Similarly, Tfbbc tiling focus intersection boundary bc 144 within image C 150, Ccb value of C at the cb border 153 is a numerical average of a multiplicity of representative pixels. Within image B 140, Bbc value of B at the bc border 143 is a numerical average of a multiplicity of representative pixels. As in Tfbab tiling focus intersection boundary ab 134 and Tfbac tiling focus intersection boundary ac 154 above, Ccb value of C at the cb border 153 may not be equal to Bbc value of B at the bc border 143 because they are two separate photographs being mated together. Δbc average pixel value difference between Bbc and Ccb 146 will be equal in magnitude to Δcb average pixel value difference between Ccb and Bbc 156 but opposite in sign.

The following example demonstrates the convergence algorithm where image A 130 has an area Aa 131 of 3 units of area, image B 140 has an area Ba 141 of 2 units of area, and image C 150 has an area Ca 151 of 1 unit of area. The composite image 129 has a total area of Aa+Ba+Ca, or 3+2+1, or 6 units of area. If Aab value of A at the ab border 132 were 155 and Bba value of B at the ba border 142 were 145, then Δab average pixel value difference between Aab and Bba 135 is 155-145, or 10 intensity units, and Aab would therefore have to decrease in value and Bba would have to increase in value so they can meet somewhere in the middle to eliminate the step in intensity value. For the first calculation in the convergence process, the Aa area of image A 131 has an area of 3 units of area and the Ba area of image B 141 has an area of 2 area units, or a combined total area of Aa+Ba, or 3+2, or 5 units of area. Image A 130 therefore comprises ⅗ or 60% of the combined A-B area, and image B 140 comprises ⅖ or 40% of the A-B area. Stated generally, A's fraction of the combined area is Aa/(Aa+Ba) and B's fraction of the combined area is Ba/(Aa+Ba). Because image A 130 has a larger area it will be altered by B's proportion of the combined area, or 40%, or 0.4×10=4 intensity units, and 155−4=151. Because image B 140 has the smaller area it will be altered by A's proportion of the combined area, or 60%, or 0.6×10=6 intensity units, and 145+6=151, so 151 becomes the new intensity value of both Aab and Bba. Because the value of Aab lowered in value, all the pixels within image A 130 also lower, and the value of Δac has also lowered by 4 intensity units or by an amount determined by the intensity and color map transform function. And because the value of Bba has raised in value, all the pixels within image B 140 also raises, and Bbc will also raise by 4 intensity units. So with every merging at a pixel focus locus boundary, all variables are redefined and given an incremented index whether they change or not.

Before any operations, the starting conditions with subscript i=0 are Aab0, Bba0, Aac0, Cca0, Ccb0, and Bbc0. And |Δab0|=|Δba0|=|Aab0−Bba0|=|Bba0−Aab0|, |Δac0|=|Δca0|=|Aac0−Cca0|=|Cca0−Aac0|, and |Δbc0|=|Δcb0|=|Bbc0−Ccb0|=|Ccb0−Bbc0|.

Operation 1: the merging of A with B at Tfbab with no effect on image C

After the 1^{st} operation with subscript i=1, the new conditions are: Aab1 has changed from Aab0, Bba1 has changed from Bba0, Aab1=Bba1, thus |Δba|=|Δab|=0, Aac1 has changed from Aac0, Cca1 still=Cca0, Ccb1 still=Ccb0, and Bbc1 has changed from Bbc0, thus |Δbc1|=|Δcb1|, which are new values as explained above and |Δac1|=|Δca1|, Aab1=(((Ba/(Aa+Ba)×Δab0)+Aab0)=Bba1=(((Aa/(Aa+Ba)×Δba0)+Bba0).

Operation 2: the merging of combined area A and B at Tfbac with area C. After the 2^{nd} operation with subscript i=2, the new conditions are: Aa=3, Ba=2, and Ca=1, and Aa+Ba+Ca=6, Aa+Ba=5, (Aa+Ba)/(Aa+Ba+Ca)=⅚=0.833=83.3%, which is the percentage amount Δca1 must move towards A, and Ca/(Aa+Ba+Ca)=⅙=0.166=16.67%, which is the percentage amount Δac1 must move towards C. Aac2=(((Ca/(Aa+Ba+Ca)×Δca1)+Aac1)=Cca2=(((Aa+Ba)/(Aa+Ba+Ca)×Δac1)+Cca1)Aab2=Bba2, Aac2=Cca2, Ccb2 and Bbc2 have changed producing a new |Δbc2| and |Δcb2|.

Operation 3: the merging of combined area A and C at Tfbbc with area B.

After the 3^{rd} operation with subscript i=3, the new conditions are: Aa=3, Ba=2, and Ca=1, and Aa+Ca=4, Ba=2, (Aa+Ca)/(Aa+Ba+Ca)= 4/6=0.666=66.67%, which is the percentage amount Δbc must move towards C, and Ba/(Aa+Ba+Ca)= 2/6=333=33.33%, which is the percentage amount Δcb must move towards B Ccb3=(((Ba/(Aa+Ba+Ca)×Δcb2)+Ccb2)=Bbc3=(((Aa+Ca)/(Aa+Ba+Ca)×Δbc12)+Bcb2)Aac3=Cca3, Ccb3=Bbc3, Bba3 and Aab3 have changed producing a new |Δba3| and |Δab3|.

Operation 4: the merging of combined area B and C at Tfbab with area A.

After the 4^{th} operation with subscript i=4, the new conditions are: Ba=2, and Ca=1, Aa=3, and Ba+Ca=3, Aa=3, (Ba+Ca)/(Aa+Ba+Ca)= 3/6 0.5=50%, which is the percentage amount Δab must move towards B, and Aa/(Aa+Ba+Ca)= 3/6 0.5=50%, which is the percentage amount Aba must move towards A, Bba4=(((Aa/(Aa+Ba+Ca)×Δba3)+Bba2)=Aab4=(((Ba+Ca)/(Aa+Ba+Ca)×Δba3)+Bba3), Aab4=Bba4, Ccb4=Bbc4, Aac4 and Ccb4 have changed producing a new Δac4.

Operation 5: same as operation 2, all subscripts of 4 increment to 5, all subscripts of 3 increment to 4.

Operation 6: same as operation 3, all subscripts of 5 increment to 6, all subscript of 4 increment to 5, and so on, with test after each operation until Δab=Δba, Δac=Δca, and Δab=Δba.

FIG. 11C depicts a donut topology of an in-focus composite image 260 composed of two in-focus sub-images that are stitched together at a continuous boundary wherein image B 271 of Ba area of B 272 is completely enclosed within image A 261 of Aa area A 262. The Aab value of A at the ab border 263 is separated from Bba value of B at the ba border 273 at the Tfbab tiling focus intersection boundary ab 264. Δab average pixel value difference between Aab and Bba 265, which is the same magnitude but opposite in sign of Δba average pixel value difference between Bba and Aab 275.

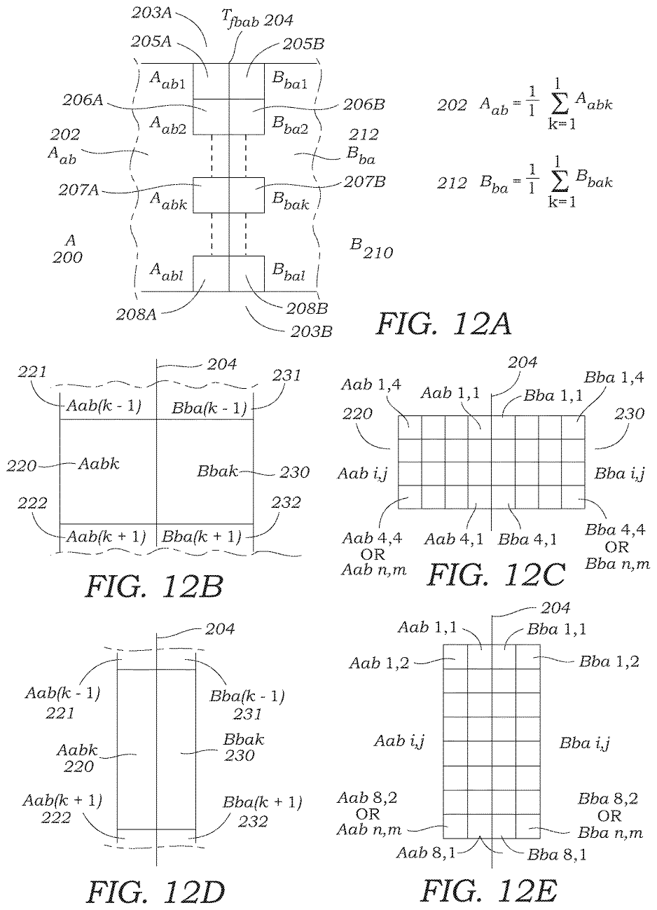

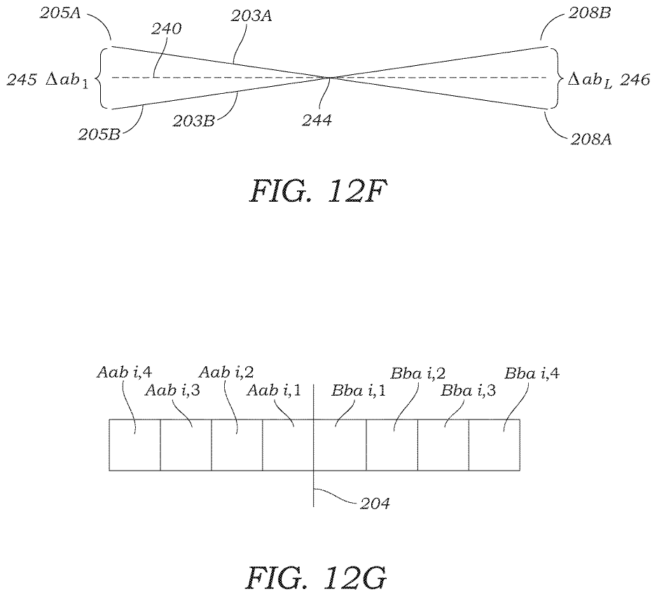

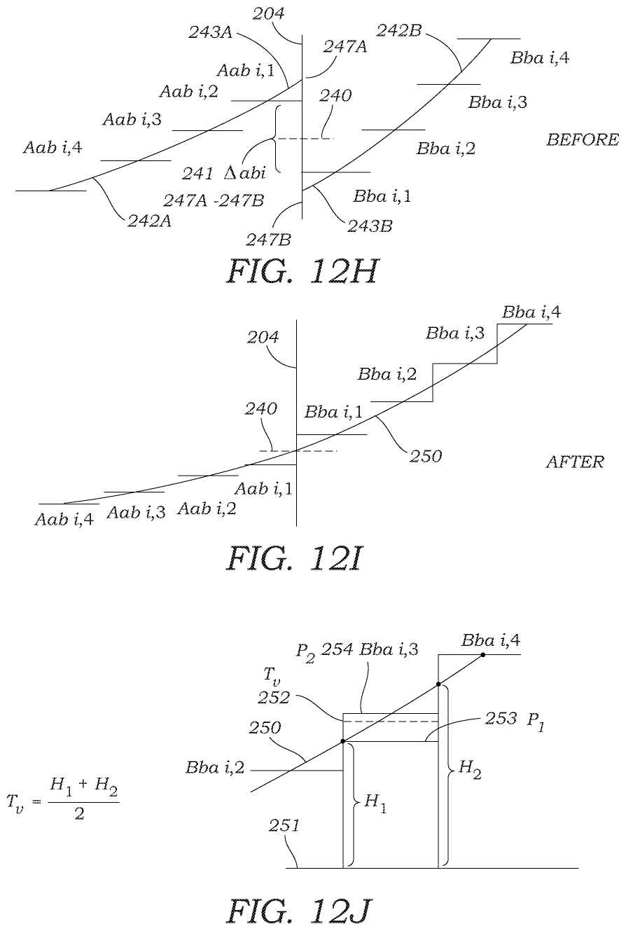

FIG. 12A depicts a closeup of a generalized Tfbab generalized tiling focus intersection boundary ab 204 which is the boundary of a generalized Aab value of A at the ab border 202 within generalized image A 200 and a generalized Bba value of B at the ba border 212 within generalized image B 210. The average brightness at a tiling focus intersection boundary is the average of all the pixel clusters at the boundary, and the brightness should be the same on both sides. To establish the average brightness, areas of pixel clusters are summed along each side of the boundary. Shown is Aab edge of interface boundary in image A 203A and Bba edge of interface boundary in image B 203B on both sides of the Tfbab generalized tiling focus intersection boundary ab 204. As shown, the generalized Aab value of A at the ab border 202 will be the sum of the first A side pixel cluster at boundary 205A, the second A side pixel cluster at boundary 206A, and so on through the Kth intermediate A side pixel cluster at boundary 207A, and finally to the Lth last A side pixel cluster at boundary 208A. There are L pixel clusters so the sum must be divided by L to obtain the average. And on the B side the generalized Bba value of B at the ba border 212 will be the sum of the first B side pixel cluster at boundary 205B, the second B side pixel cluster at boundary 206B, and so on through the Kth intermediate B side pixel cluster at boundary 207B, and finally to the Lth last B side pixel cluster at boundary 208B. And likewise there are L pixel clusters so the sum must be divided by L to obtain the average. Generally stated:

$$Aab = 1 / L {\sum }_{k = 1}^{l} Aabk and Bba = 1 / L {\sum }_{k = 1}^{l} Bbak$$

FIG. 12B depicts a closeup view of a square pixel cluster comprised of 16 pixels. Shown is a closeup view of the Kth pixel cluster on both sides of a generalized Tfbab generalized tiling focus intersection boundary ab 204. On the A side is shown the generalized Kth intermediate A side pixel cluster at boundary 220, the generalized (K−1)th intermediate A side pixel cluster at boundary 221 which is the pixel cluster before, and the generalized (K+1)th intermediate A side pixel cluster at boundary 222, which is the pixel cluster after. Likewise, on the B side is shown the generalized Kth intermediate B side pixel cluster at boundary 230, the generalized (K−1)th intermediate B side pixel cluster at boundary 231 which is the pixel cluster before, and the generalized (K+1)th intermediate B side pixel cluster at boundary 232, which is the pixel cluster after. Also, each A subscript pixel cluster is opposite the B subscript pixel cluster on the other side of the focus locus boundary.

FIG. 12C depicts one possible 16 pixel example of the generalized Kth intermediate A side pixel cluster at boundary 220 and the generalized Kth intermediate B side pixel cluster at boundary 230 depicted in FIG. 12B. The generalized Kth intermediate A side pixel cluster at boundary 220 is composed of 16 pixels arranged in four rows and four columns. In general there can be n rows and m columns. The designation of an individual pixel within its respective pixel cluster is Aabi,j, where the i subscript goes from 1 to n where n is the maximum number of rows, and the j subscript goes from 1 to m where the maximum number of columns, and thus there are n×m number of pixels in the pixel cluster. Likewise, the B side is a mirror image of the A side, with the designation of an individual pixel within its respective pixel cluster is Bbai,j, where the i subscript goes from 1 to n where n is the maximum number of rows, and the j subscript goes from 1 to m where m is the maximum number of column, and thus there are n×m number of pixels. Note that the column index j increases as the pixels move further from the Tfbab generalized tiling focus intersection boundary ab 204. The j index is 1 at the column on the Tfbab generalized tiling focus intersection boundary ab 204. Generally stated:

$$Aabk = 1 / nm {\sum }_{i = 1}^{n} {\sum }_{j = 1}^{m} Aabi , j and$$

$$Babk = 1 / nm {\sum }_{i = 1}^{n} {\sum }_{j = 1}^{m} Bbai , j$$

In the example of 12B and 12C n=4 and m=4, thus there are 16 pixels. 16 pixels are shown for example only and the process is not limited to both a square shape or 16 pixels.

FIG. 12D depicts a closeup view of a rectangular pixel cluster comprised of 16 pixels. Shown is a closeup view of the Kth pixel cluster on both sides of a generalized Tfbab generalized tiling focus intersection boundary ab 204. On the A side is shown the generalized Kth intermediate A side pixel cluster at boundary 220, the generalized (K−1)th intermediate A side pixel cluster at boundary 221 which is the pixel cluster before, and the generalized (K+1)th intermediate A side pixel cluster at boundary 222, which is the pixel cluster after. Likewise, on the B side is shown the generalized Kth intermediate B side pixel cluster at boundary 230, the generalized (K−1)th intermediate B side pixel cluster at boundary 231 which is the pixel cluster before, and the generalized (K+1)th intermediate B side pixel cluster at boundary 232, which is the pixel cluster after. Also, each A subscript pixel cluster is opposite the B subscript pixel cluster on the other side of the focus locus boundary.

FIG. 12E depicts one possible 16 pixel example of the generalized Kth intermediate A side pixel cluster at boundary 220 and the generalized Kth intermediate B side pixel cluster at boundary 230 depicted in FIG. 12B. The indexes i and j are the same as those in FIG. 12C except that n, the maximum number of rows=8 and m, the maximum number of columns is 2. The indexing and summation is the same, and thus

$$Aabk = 1 / nm {\sum }_{i = 1}^{n} {\sum }_{j = 1}^{m} Aabi , j and$$

$$Babl = 1 / nm {\sum }_{i = 1}^{n} {\sum }_{j = 1}^{m} Bbai , j$$

where n=8 and m=2, and n×m=16. 16 pixels are shown for example only and the process is not limited to both a rectangular shape or 16 pixels.

FIG. 12F depicts a generalized Tfbab generalized tiling focus intersection boundary ab 204 as shown in FIG. 12A. Aab edge of interface boundary in image A 203A covers the entire generalized Tfbab generalized tiling focus intersection boundary ab 204 from the first A side pixel cluster at boundary 205A to Lth last A side pixel cluster at boundary 208A. Likewise, Bba edge of interface boundary in image B 203B covers the entire generalized Tfbab generalized tiling focus intersection boundary ab 204 from the first B side pixel cluster at boundary 205B to Lth last B side pixel cluster at boundary 208B. Because Aab and Bba are averages, they may not be equal at many locations along their entire length. As shown, Aab edge of interface boundary in image A 203A only equals Bba edge of interface boundary in image B 203B at the spot where 203A=203B 244. As one moves from this equal point in either direction the differences grow as shown by Δab1=theoretical delta at the first interface Aab1,1-Bba1,1 245 at the first pixel cluster and ΔabL=theoretical delta at the first interface AabL,1-BbaL,1 246 at the Lth pixel cluster.

FIG. 12G shows four A image pixels adjoining four B image pixels along the ith row at the Tfbab generalized tiling focus intersection boundary ab 204. As shown, Aabi,1 abuts with Bbai,1. Moving outward in the A direction, Aabi,2 is next to Aabi,1, followed by Aabi,3, then Aabi,4 farthest away from the tiling focus boundary. And moving outward in the B direction, Bbai,2 is next to Bbai,1, followed by Bbai,3, then Bbai,4 farthest away from the tiling focus boundary.

FIG. 12H depicts a typical representation of the values of eight pixels shown in FIG. 12G. Shown is Tfbab generalized tiling focus intersection boundary ab 204. The theoretical Aabi curve of higher resolution derived from quantized intensities 242A is a best fit curve formed from pixels Aabi,1, Aabi,2, Aabi,3, and Aabi,4. This curve is the result of higher precision arithmetic than the quantized values of the individual pixels. The slope of the theoretical Aabi curve of higher resolution derived from quantized intensities 242A at the Tfbab generalized tiling focus intersection boundary ab 204 is slope of theoretical Aabi curve at the AB tiling border 243A. The theoretical Bbai curve of higher resolution derived from quantized intensities 242B is a best fit curve formed from pixels Bbai,1, Bbai,2, Bbai,3, and Bbai,4. This curve is also the result of higher precision arithmetic than the quantized values of the individual pixels. The slope of the theoretical Bbbi curve of higher resolution derived from quantized intensities 242B at the Tfbab generalized tiling focus intersection boundary ab 204 is slope of theoretical Bbai curve at the BA tiling border 243B. Shown is the point where 243A intersects 204 (247A) and the point where 243B intersects 204 (247B), and thus Δabi=theoretical delta at the ith interface 247A-247B 241. The theoretical AB curve match line for a given ith location 240 is the target location where modified theoretical Aabi curve of higher resolution derived from quantized intensities 242A and modified theoretical Bbai curve of higher resolution derived from quantized intensities 242B must center.

FIG. 12I depicts the merging process of the two curves depicted in FIG. 12H. The values of Aabi,4 and Bbai,4 are maintained as end points and the theoretical AB curve match line for a given ith location 240 define a new theoretical derived curve merging 242A with 242 B about 240 (250), and on the A side this defines new values for Aabi,1, Aabi,2, and Aabi,3, and on the B side this defines new values for Bbai,1, Bbai,2, and Bbai,3. And this process is repeated for all values of i between 1 and L. The fixed-point numerical values must be determined and is the rounded either up or down based on the new theoretical derived curve merging 242A with 242 B about 240 (250) as explained in FIG. 12J.

FIG. 12J shows how a quantified individual pixel value is established from a higher resolution theoretical target value. Shown is an enlargement of pixel Bbai,3, with Bbai,2 to the left and Bbai,4 to the right. Shown is the new theoretical derived curve merging 242A with 242 B about 240 (250), and a trapezoid is formed by H1, H2, the portion of the new theoretical derived curve merging 242A with 242 B about 240 (250) which is over the Bbai,3 pixel region, and the 0 datum 251. The average pixel height theoretical value Tv=(H1+H2)/2, where H1=value at lower pixel edge and H2=value at upper pixel edge 252, which is represented by the dotted line. As can be seen, the dotted line is bounded by the P1 rounded down value 253 and the P2=Bbai,3, rounded up value 254, which is closer numerically to the theoretical value, so this is the value chosen.

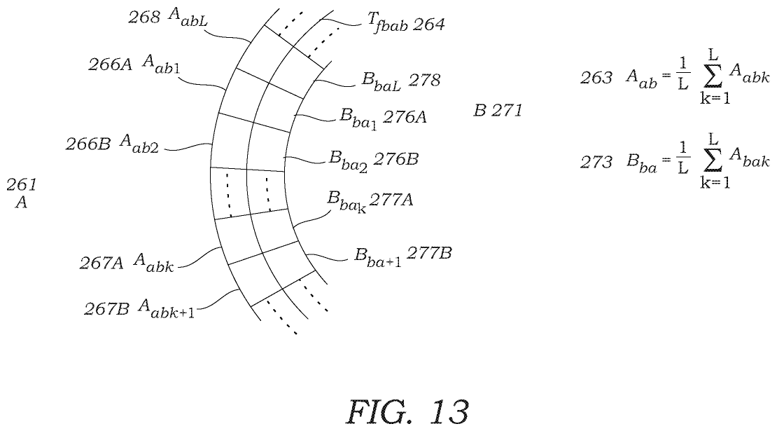

FIG. 13 depicts a closeup of Tfbab tiling focus intersection boundary ab 264 which is the boundary that separates the Aab value of A at the ab border 263 from the Bba value of B at the ba border 273. The average brightness at a tiling focus intersection boundary is the average of all the pixel clusters at the boundary, and the brightness should be the same on both sides. To establish the average brightness, areas of pixel clusters are summed along each side of the boundary. As shown, the generalized Aab value of A at the ab border 263 will be the sum of Aab1, the 1^{st} intermediate A side pixel cluster at boundary 266A, Aab2, the 2^{nd} intermediate A side pixel cluster at boundary 266B, on through Aabk, the Kth intermediate A side pixel cluster at boundary 267A, the Aabk+1, the K+1th intermediate A side pixel cluster at boundary 267B, and finally AabL, the Lth intermediate A side pixel cluster at boundary 268 which is adjacent to Aab1, the 1^{st} intermediate A side pixel cluster at boundary 266A because all these pixel clusters are topologically a toroid. There are L pixel clusters so the sum must be divided by L to obtain the average. And on the B side the generalized Bba value of B at the ba border 273 will be the sum of the Bba1, the 1^{st} intermediate B side pixel cluster at boundary 276A, Bba2, the 2^{nd} intermediate B side pixel cluster at boundary 276B, on through Bbak, the Kth intermediate B side pixel cluster at boundary 277A, Bbak+1, the K+1th intermediate B side pixel cluster at boundary 277B, and finally BbaL, the Lth intermediate B side pixel cluster at boundary 278, which is adjacent to Bba1, the 1^{st} intermediate B side pixel cluster at boundary 276A because all these pixel clusters are topologically a toroid. There are L pixel clusters so the sum must be divided by L to obtain the average. Generally stated:

$$Aab = 1 / L {\sum }_{k = 1}^{l} Aabk and Bba = 1 / L {\sum }_{k = 1}^{l} Bbak$$

It should be noted that the pixels depicted in FIG. 13 are not rectangular as might be acquired from a digital camera and displayed on a screen. These non-rectangular pixels will herein be referred to as parametric pixels. Digital cameras take pictures using a rectangular sensor grid. Irregular boundaries such as curved boundaries intersect a rectangular grid at many angles, with part of a specific pixel on one side of a boundary and part of the same pixel on the other. In this case an overlaying grid of non-rectangular parametric pixels can be applied at a curved boundary such as a focus locus boundary, and each parametric pixel can be the resultant average intensity and color value of all rectangular pixels intersecting the parametric pixel. So if parametric pixel PPi,j of area APPi,j in a parametric coordinate system were composed of rectangular pixels RPx,y of respective area percentage ARPx,y overlapping or underlapping parametric pixel PPi,j, in the rectangular coordinate system of the rectangular sensor grid, a number of rectangular pixels would intersect the parametric pixel.

$${PP}_{m} = {\sum }_{k = 1}^{n} ( {RP}_{k} ) \times ( {ARP}_{k} ) / ARP or$$

$${PP}_{m} = \frac{1}{ARP} {\sum }_{k = 1}^{n} ( {RP}_{k} ) \times ( {ARP}_{k} )$$

where PPₘ is intensity or color value of the mth parametric pixel mapped from n number of rectangular pixels, k is the pixel index of the kth component of rectangular pixels, n is the total number of rectangular pixels intersecting the area of the mth parametric pixel, RPk is intensity or color value of the kth rectangular pixel intersection of the area of the parametric pixel, ARPₖ is the area of the kth rectangular pixel intersecting the area of the parametric pixel, and ARP is the area of a rectangular pixel.

The calculations are of higher resolution than the resolution of either the rectangular sensor grid or any parametric pixels so that error introduction is minimized.

A mapped transform can go from the rectangular pixels of the rectangular sensor grid to the parametric pixels of the parametric space. Calculations and intensity and color alterations can then occur acting upon the parametric pixels within the parametric pixel space, then transformed back from the parametric pixel space to the rectangular grid.

$${RP}_{p} = {\sum }_{r = 1}^{s} ( {PP}_{r} ) \times ( {APP}_{r} ) / APP or$$

$${RP}_{p} = \frac{1}{APP} {\sum }_{r = 1}^{s} ( {PP}_{r} ) \times ( {APP}_{r} )$$

where RPₚ is intensity or color value of the pth rectangular pixel mapped from r number of parametric pixels, r is the pixel index of the rth component of parametric pixels, s is the total number of parametric pixels intersecting the area of the pth rectangular pixel, PPr is intensity or color value of the rth parametric pixel intersection the of area of the rectangular pixel, APPᵣ is the area of the rth parametric pixel intersecting the area of the rectangular pixel, and APP is the area of a parametric pixel.

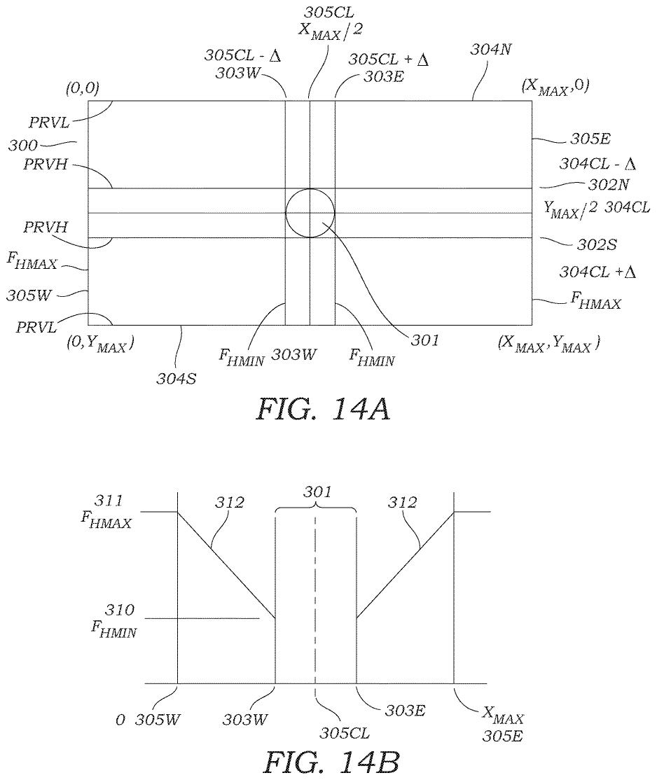

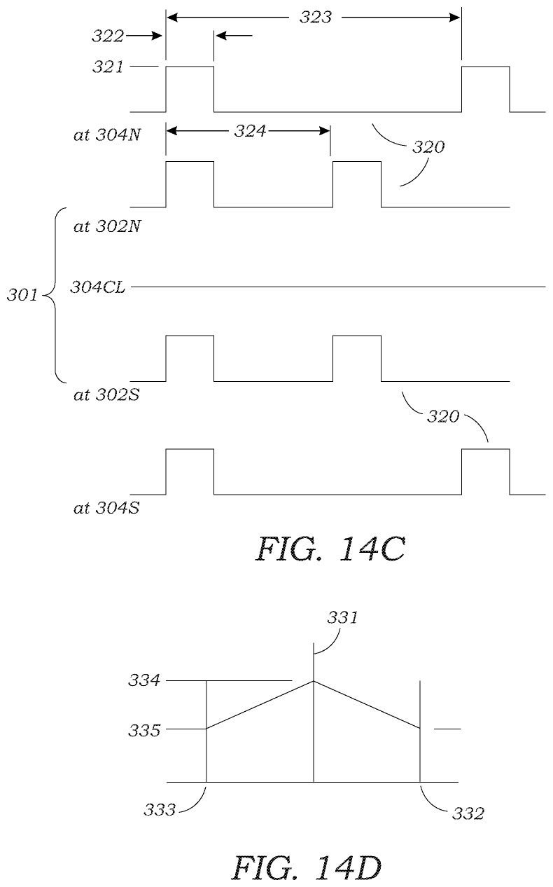

FIG. 14A depicts a display screen displaying what the camera sees, and the display shows the zones affecting the sounds produced as a function of the phone camera's position relative to an eye or target. In this example and for some phones, the origin is located in the upper left corner of the screen, and using standard Cartesian coordinate notation, the origin is designated as 0, 0. The X coordinate is the horizontal axis and the Y coordinate is the vertical axis. The screen is bounded by the leftmost vertical edge of display 305W to the left or west, the northmost horizontal edge of display 304N at the top or north, the rightmost vertical edge of display 305E to the right or east, and the southmost horizontal edge of display 304S at the bottom or south. The top of the screen has a vertical component value of 0 and a horizontal component of X. The upper right corner of the screen is Xmax, which is the maximum number of pixels defined by the camera's resolution, and this corner would be designated as Xmax, 0. The lower left corner has a horizontal component of 0 and a vertical component of Y. The lower left corner of the screen is Ymax, which is the maximum number of pixels defined by the camera's resolution, and this corner would be designated as 0, Ymax. The lower right corner of the screen is therefore Xmax, Ymax.

The vertical center line of the centered zone 305CL is located at Xmax/2 and the horizontal center line of the centered zone 304CL is located at Ymax/2. If A is the radius of the zone of centered position both horizontally and vertically 301, then the vertical center line of the centered zone 305CL-A is the westmost vertical edge of the centered zone 303W, the vertical center line of the centered zone 305CL+A is the eastmost vertical edge of the centered zone 303E, the northmost horizontal edge of the centered zone 302N-A is the northmost horizontal edge of the centered zone 302N, and the southmost horizontal edge of the centered zone 302S+A is the southmost horizontal edge of the centered zone 302S.

FIG. 14B depicts a first sound which is a horizontal frequency 312 that changes as the target or eye moves horizontally across from off the display screen to the left or west across the screen to off the display screen to the right or east. Off the screen to the left the phone produces an easily audible audio tone of FHMAX, or maximum horizontal frequency 311. Since the camera doesn't know where the phone is if there is no target anywhere on the camera display area 300, this sound will be produced until the target or eye is within the camera display area 300. As the target or eye moves in the positive X direction to the right of leftmost vertical edge of display 305W, the horizontal frequency 312 decreases to signal to the user that the phone is being both tilted and translated in the correct direction to center the camera over the target or eye. When at the westmost vertical edge of the centered zone 303W the FHMIN, or minimum horizontal frequency 310 is being produces. Once entering zone of centered position both horizontally and vertically 301 the horizontal frequency 312 drops to zero and no sound is produced. There is no horizontal frequency 312 as the target passes across the zone of centered position both horizontally and vertically 301 until crossing the eastmost vertical edge of the centered zone 303E, at which time FHMIN, or minimum horizontal frequency 310 is produced, and as positive X motion continues the horizontal frequency 312 continues to increase until touching the eastmost vertical edge of the centered zone 303E, at which time the horizontal frequency 312 is FHMAX, or maximum horizontal frequency 311, and continues to any place to the right or east and off the display screen. This is one convention of producing audio feedback to drive the user to the centered position. The convention of increasing frequency as one moves further off center is only one example of a changing audio signal that can guide the user back to center whether off center to the right or left. When the sound stops the camera is centered horizontally.

FIG. 14C depicts a second sound that is a pulse train which changes its period as a function of the vertical position, and this provides feedback to a user to center a phone's camera to be centered over a target or eye. In this embodiment pulse waveform 320 is produced if the phone is outside and beyond the northmost horizontal edge of display 304N. Since the camera doesn't know where the phone is if there is no target anywhere on the camera display area 300, this sound will be produced until the target or eye is within the camera display area 300. When the cameral is positioned at the northmost horizontal edge of display 304N, pulse waveform 320 produces pulse repetition period vertical low 323. This waveform is composed of a pulse on frequency 321 for a pulse on duration 322, and a pulse repetition period vertical low 323 as shown in FIG. 14C. As the camera proceeds in the positive Y direction and downward, the pulse repetition rate increases until at the northmost horizontal edge of the centered zone 302N where the pulse repetition period vertical high 324 is produced. When entering the zone of centered position both horizontally and vertically 301 the pulse waveform 320 ceases and there is no contribution of this component of sound. Continuing in the positive Y downward direction when at the southmost horizontal edge of the centered zone 302S the pulse repetition period vertical high 324 is produced. The pulse repetition period increases and thus the pulse repetition rate decreases as the target or eye approaches the southmost horizontal edge of display 304S where the pulse repetition period vertical low 323 is produced. Should the camera continue off the screen the pulse repetition period vertical low 323 will persist until the target or eye returns to the display area.

FIG. 14D depicts a third sound that is used to provide guiding distance feedback after the camera is centered over the target or eye is in the center of the camera display area 300. The optimal centered position is when the target or eye is at the intersection of the horizontal center line of the centered zone 304CL and the vertical center line of the centered zone 305CL. When the phone is in the zone of centered position both horizontally and vertically 301, both the horizontal frequency 312 and the pulse waveform 320 are no longer producing sound. When this condition is met, the third sound indicating distance to target 330 commences. As long as the phone remains centered, this third sound indicating distance to target 330 plays continuously. When the phone is at the optimal distance to target 331 the phone plays the frequency at optimal distance 334. In this embodiment as indicated in FIG. 14D the frequency is at a maximum. As the phone moves away from the optimal distance to target 331 the frequency changes, which in this embodiment drops. When the distance is too close 333 or the distance is too far 332 the frequency at maximum displacement from optimal distance 335 drops. This provides feedback to the user to maximize the frequency and maintain that distance while the phone takes at least one photo of the target or eye. If the phone moves out of the zone of centered position both horizontally and vertically 301, the third sound indicating distance to target 330 stops and is replaced by either the horizontal frequency 312 or the pulse waveform 320 or both, whichever is applicable to guide the user back to the zone of centered position both horizontally and vertically 301, and then the third sound indicating distance to target 330 resumes.

FIG. 15A depicts the CIE chromaticity chart 348, which depicts all colors perceivable by the human eye. The chart has an X axis and a Y axis, and each wavelength of light can be located along the perimeter of bounded area with a corresponding X and Y coordinate. Contained within and a subset of the CIE chromaticity chart 348 is the triangle of colors that can be matched by R, B, G 350, where R is red 350R, G is green 350G, and B is blue 350B. Located somewhere within the center of the triangle of colors that can be matched by R, B, G 350 is the E true achromatic point 351, also known as CIE white.

A camera has a blue filter to detect the blue component of light upon a light sensor in an array, a green filter to detect the green component of light upon a light sensor in an array, and a red filter to detect the red component of light upon a light sensor in an array. Filters vary in the peak wavelength of light detected, and the sensing element that measures the light level passing through each respective filter varies as well. While manufacturers strive to create repeatability there are variations in both peak filter wavelength and sensitivity perception by the light sensing elements, and there are variations from manufacturer to manufacturer.

Color correction compensates for variations in camera color perception of red, blue, and green camera filters and light level sensitivity quantization, which can vary from camera to camera and manufacturer to manufacturer. Color correction also compensates for the spectral composition of the illuminating light source which can be anything from including but not limited to natural sunlight, incandescent light, halogen light, LED lighting, fluorescent lighting, lighting panels, or any other light source now known or unknown.

The same holds true for an image display, which is produced by red, blue, and green LEDs. The same image can vary from display to display and from manufacturer to manufacturer. The display is unnecessary for diagnostic purposes however, only useful for human viewing. For diagnostics the system is only concerned with what the camera sees and how the system subsequently process the image to maximize its diagnostic usefulness.

FIG. 15B duplicates the triangle of colors that can be matched by R, B, G 350, and depicts the application of color correction to a camera's raw image or composite image. In FIG. 15B red 350R is a representative red wavelength, but the distribution of red wavelengths at this vertex of the triangle of colors that can be matched by R, B, G 350 is a cluster of red frequencies 352R. Green 350G is a representative green wavelength, but the distribution of green wavelengths at this vertex of the triangle of colors that can be matched by R, B, G 350 is a cluster of green frequencies 352G. And likewise, blue 350B is a representative blue wavelength, but the distribution of blue wavelengths at this vertex of the triangle of colors that can be matched by R, B, G 350 is a cluster of blue frequencies 352B. It is therefore necessary to color correct the generic composite image 340, which is either composite image 169 of FIG. 11A, the composite image 129 of FIG. 11B, or composite image 260 of FIG. 11C.

The generic composite image 340 in the raw digital red 350R, blue 350B, and green 350G is influenced by the spectrum of the illumination source, the frequency of the filters for each respective color, and the sensitivity of the light sensing elements themselves at each pixel location. Thus, after the stitching process, if necessary, the generic composite image 340 may require a color correction transform fcc(Xc, Yc) 360 applied to each color of each pixel to normalize the image. Each pixel has a spatial X and Y coordinate within the matrix of the generic composite image 340. The raw image, generic composite image 340 can in shorthand notation be represented as the raw color camera values of spatial image I(Xc, Yc) 354, where each pixel has three component colors associated. In this case Xc really spans from 1 to Xmax number of columns and Y spans from 1 to Ymax number of rows, thus representing a total of (Xmax) (Ymax) number of red pixels, blue pixels, and green pixels, each of which is color-mapped to different values after the color correction transform fcc(Xc, Yc) 360 is applied.

This color correction transform fcc(Xc, Yc) 360 can be experimentally determined by creating similar illumination to what would be used to take a photo of the target or an eye, and the camera can take photos of known color swatches of known color coordinates on the CIE chromaticity chart 348, and the colors perceived by the camera can be mapped from what the camera sees them as to what their absolute values should be under ideal lighting spectral conditions, and thus the color correction transform fcc(Xc, Yc) 360 can be established and applied to the generic composite image 340, and the transform can act upon I(Xc, Yc) 354 to yield the color correction transformed values (Xcc, Ycc) 360V. In this manner camera image 1^{st} pixel Xc1, Yc1 355A transforms into color corrected 1^{st} pixel Xcc1, Ycc1 355B, camera image ith pixel Xci, Yci 356A transforms into color corrected ith pixel Xcci, Ycci 356B, camera image nth pixel Xcn, Ycn 357A transforms into color corrected nth pixel Xccn, Yccn 357B, and camera image CIE white Ec, or Xce, Yce 358A transforms into color corrected CIE white Ecc, or Xcce, Ycce 358B.

FIG. 15C depicts the application of color conditioning to increase the sensitivity of machine based discrimination between having and not having a condition of interest. Shown is the application of color conditioning transform fcct(Xcc, Ycc) 361 to the color correction transformed values (Xcc, Ycc) 360V of FIG. 15B to yield the color conditioning transform values (Xcct, Ycct) 361V. The input values for the application of color conditioning transform fcct(Xcc, Ycc) 361 are the color correction transformed values (Xcc, Ycc) 360V generated by the color correction transform fcc(Xc, Yc) 360 as depicted in FIG. 15B.

Color corrected 1^{st} pixel Xcc1, Ycc1 355B becomes color condition transformed 1^{st} pixel Xcct1, Ycct1 355C, color corrected ith pixel Xcci, Ycci 356B becomes color condition transformed ith pixel Xccti, Yccti 356C, color corrected nth pixel Xccn, Yccn 357B becomes color condition transformed nth pixel Xcctn, Ycctn 357C, and finally color corrected CIE white Ecc, or Xcce, Ycce 358B becomes color condition transformed CIE white Ecct, or Xccte, Yccte 358C.

Application of the color conditioning transform fcct(Xcc, Ycc) 361 is to improve infection, medical condition, or impairment detection sensitivity. Because different conditionings may facilitate easier discrimination for different conditions such as Covid, SARS, Zika, diabetic retinopathy malaria or any infection now known or unknown, the application of the right color conditioning transform may raise the confidence level of machine generated diagnostics and lower the percentage of false negatives and false positives.

A summary of the starting image, the color corrected image, and the color conditioned image as described in FIG. 15B and FIG. 15C:

| | |
|---|---|
| l(Xc, Yc) = raw color camera values of spatial image | 110 in FIG. 10 |
| fcc(Xc, Yc) = (Xcc, Ycc) color correction values | 115 in FIG. 10 |
| fcct(Xcc, Ycc) = (Xcct, Ycct) color condition transform | 116 in FIG. 10 |

| composite | | color | | color |
|---|---|---|---|---|
| image from | | correction | | condition |
| camera | | transform | | transform |
| A | | B | | C |
| l(Xc, Yc) | → | fcc(Xc, Yc) | → | fcct(Xcc, Ycc) |
| Xc1, Yc1 | → | Xcc1, Ycc1 | → | Xcct1, Ycct1 |
| Xci, Yci | → | Xcci, Ycci | → | Xccti, Yccti |
| Xcn, Ycn | → | Xccn, Yccn | → | Xcctn, Ycctn |
| Ec = Xce, Yce | → | Ecc = Xcce, Ycce | → | Ecct = Xccte, Yccte |

Ec = achromatic point (358A)
Ecc = true color correction achromatic point (358B)
Ecct = color condition transformed point (358C)

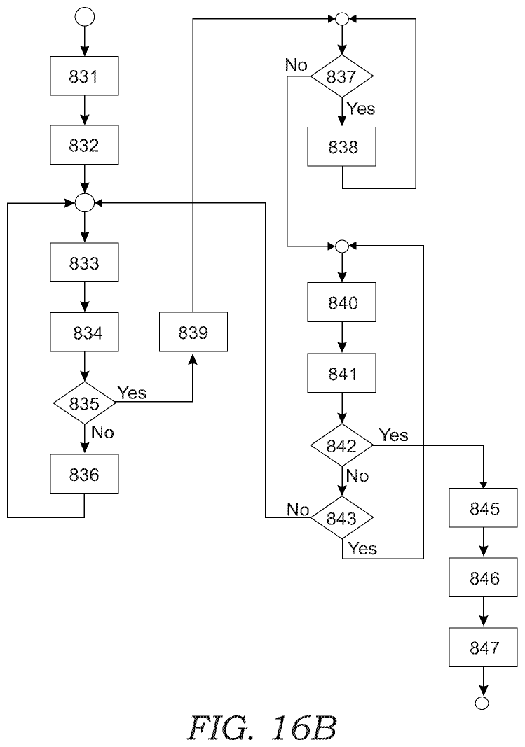

FIG. 16A is a flowchart defining steps necessary to generate three components of positional feedback to optimally position a phone camera and to take pictures of a target or eye. As defined earlier, when the phone is both translated along the X axis and rotated about the Y axis this produces displacement along the X axis of the phone's camera and also where a target or eye is positioned within the display. As per FIGS. 14A and 14B the phone produces a first sound proportional to X position. And when the phone is both translated along the Y axis and rotated about the X axis this produces displacement along the Y axis of the phone's camera and also where a target or eye is positioned within the display. As per FIGS. 14A and 14C the phone produces a second sound proportional to Y position.

Referring to FIG. 16A camera positioning and taking photos, two X-Y feedback sounds 800, the first step is video ON to establish position and face camera forward 801, followed by reset photo index (PI) to 0 802 and 3^{rd} sound off 803. The next step is to locate target or eye and establish X and Y values 804. These values are used to process the first and second sounds to be used for user feedback guidance. The test must be performed: is X in central zone? 805. If X is not within the central zone then the phone will generate 1^{st} sound proportional to position 806. This first sound will be proportional to the absolute value of difference between the central zone and east and west image boundaries as outlined in FIG. 14A. In this case the further X is to the right of the central zone and the further X is to the left of the central zone the greater will be the change of a parameter such as frequency to guide the user in a restoring direction toward the central zone. If X is within the central zone then no first sound needs to be produced and the next step is insure 1^{st} sound off 811 followed by evaluating: is Y in central zone? 807.

If Y is not within the central zone then the phone will generate 2^{nd} sound proportional to position 808. This second sound will be proportional to the absolute value of difference between the central zone and the north and south image boundaries as outlined in FIG. 14A. In this case the further Y is above the central zone and the further Y is below the central zone the greater will be the change of a parameter such as pulse repetition rate to guide the user in a restoring direction toward the central zone. If Y is within the central zone then no second sound needs to be produced, or 2^{nd} sound off 812. In this manner the first sound and the second sound guides the user to center the phone's camera. When centered both the first sound and second sounds are off, then the distance between the camera and the target is evaluated in the step is Z> or <optimal distance? 809, where Z is the distance between the camera and the target or eye. The phone will then generate 3^{rd} sound proportional to camera distance to eye 810. This third sound proportional to the absolute value of difference between camera distance from target or eye and optimal distance to target or eye as depicted in in FIG. 14D. As the third sound is continuously modified the program continues to test is Z> or <optimal distance? 809. When the camera is at the optimal distance the phone will then take a photo 820, which is followed by increment photo index PI 821. The photo index counts the number of pictures taken. PImax is the maximum number of photos to be taken in rapid succession or cumulatively. The next test is PI>=PImax? 822, which determines if more photos need to be taken. After each photo is taken the phone must continue to test is X in central zone? 823 and is Y in central zone? 824. If during the course of photos being taken the camera moves outside the central zone the phone requires repositioning and the third sound stops and either the first sound or the second sound or the first and second sounds are produced until the phone is centered once again, and then both first and second sounds are shut off and replaced by the third sound to reevaluate the distance between the camera and the target or eye. When all conditions are met more photos are taken until the PImax number of photos is taken, at which time the third sound is extinguished via 3^{rd} sound off 825, followed by reset photo index (PI) to 0 826, and finally pass PImax photos to focus process 827, which is completely in focus 71, partially in focus 72, completely in focus 76, partially in focus 77, and completely in focus 91, partially in focus 92, as depicted in FIG. 10.

FIG. 16B is a flowchart defining steps necessary to generate two components of positional feedback to optimally position a phone camera and to take pictures of a target or eye. As defined earlier, when the phone is both translated along the X axis and rotated about the Y axis this produces displacement along the X axis of the phone's camera and also where a target or eye is positioned within the display. And when the phone is both translated along the Y axis and rotated about the X axis this produces displacement along the Y axis of the phone's camera and also where a target or eye is positioned within the display. In this embodiment both X and Y displacements are combined to produce a single first sound to guide the user into the zone of centered position both horizontally and vertically 301, or simply the central zone. This can be quantified in any number of ways including but not limited to taking the absolute value of difference between central zone and east and west sides+the absolute value of difference between central zone and north and south sides. The first sound can be a frequency or a pulse train of varying pulse repetition rate or other sounds that change in ways to guide the user toward the central zone.

Referring to FIG. 16B camera positioning and taking photos, one X-Y feedback sound 830, the first step is video ON to establish position and face camera forward 831, followed by reset photo index (PI) to 0 832 and 2^{nd} sound off 833. The next step is to locate target or eye and establish X and Y values 834. These values are used to process the first and second sounds to be used for feedback guidance. The test must be performed is X and Y in central zone? 835. If X or Y or both are not within the central zone then the phone will generate 1^{st} sound proportional to X and Y positions 836. As stated, this can be absolute value of difference between central zone and east and west sides+absolute value of difference between central zone and north and south sides. Other combinations and methods of combining X and Y in both linear and non-linear ways can be employed without departing from the spirit of the imaging system.

This first sound will be proportional to any deviation from the central zone. If X and Y are both within the central zone then no first sound needs to be produced and the next step is insure 1^{st} sound off 839. In this manner the first sound guides the user to center the phone's camera. When centered the first sound is off, then the distance between the camera and the target or eye is evaluated in the step is Z> or <optimal distance? 837, where Z is the distance between the camera and the target or eye. The phone will then generate 2^{nd} sound proportional to camera distance to eye 838.

This second sound proportional to the absolute value of difference between camera distance from target or eye and optimal distance to target or eye as depicted in in FIG. 14D. As the second sound is continuously modified the program continues to test is Z> or <optimal distance? 837. When the camera is at the optimal distance the phone will then take a photo 840, which is followed by increment photo index PI 841. The photo index counts the number of pictures taken. PImax is the maximum number of photos to be taken in rapid succession or cumulatively. The next test is PI>=PImax? 842 and determines if more photos need to be taken. After each photo is taken the phone must continue to test is X and Y in central zone? 843. If, during the course of photos being taken the camera moves outside the central zone the phone requires repositioning and the second sound stops and the first sound resumes until the phone is centered once again, and then the first sound is shut off and replaced by the second sound to reevaluate the distance between the camera and the target or eye. When all conditions are met more photos are taken until the PImax number of photos is taken, at which time the second sound is extinguished via 2^{nd} sound off 845, followed by reset PI to 0 846, and finally pass PImax photos to focus process 847, which is completely in focus 71, partially in focus 72, completely in focus 76, partially in focus 77, and completely in focus 91, partially in focus 92, as depicted in FIG. 10.

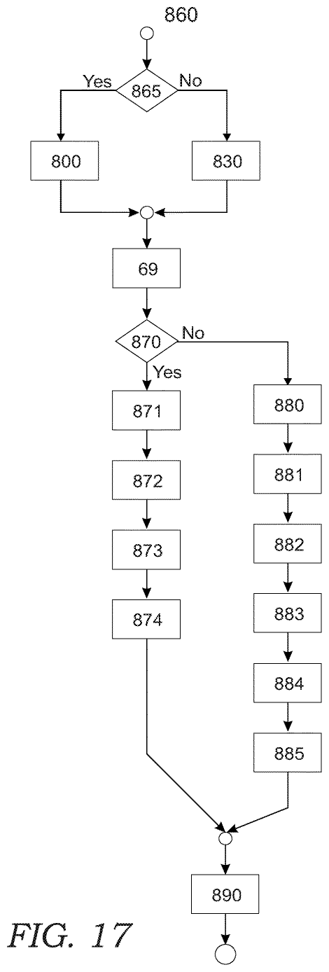

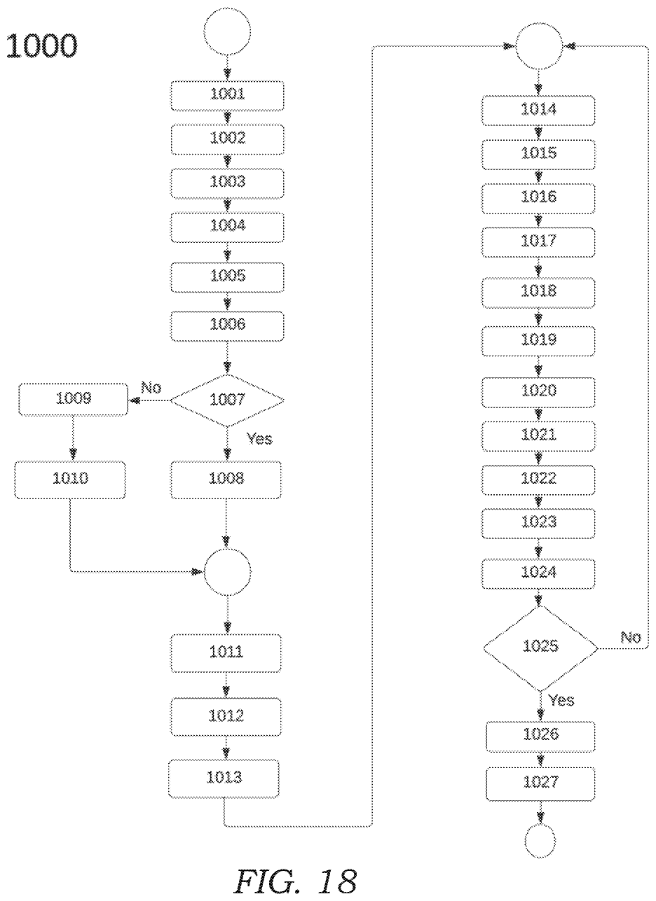

FIG. 17 depicts the global image process that incorporates positioning with the aid of audio feedback a camera to take a series of photos as depicted in FIGS. 16A and 16B, determining that at least one photograph or composite photograph is in focus, color correcting, and color conditioning as depicted in FIG. 10, then employing machine learning to compare at least one image to a first set of images possessing the shared characteristics of being representative of a condition and comparing that same image to a second set of images possessing a shared characteristic of not possessing the same condition to determine whether at least one image does or doesn't belong to the set possessing shared characteristics along with an associated confidence level for belonging to that set.

To position the phone directly over the target or eye and then at the correct distance from the target or eye the first audio guidance feedback decision is: are there two X-Y sounds? 865. If there are two sounds then a user performs camera positioning and taking photos, two X-Y feedback sounds 800, and if not the user performs camera positioning and taking photos, one X-Y feedback sound 830. Whether one or two feedback sounds are used to guide a user to position the camera properly, either process will produce a number of photos to be analyzed by creating an in-focus image or composite image 69, which is really at least one in-focus image or composite image. This step will yield at least one in-focus photograph that is cropped, color corrected, and color conditioned in preparation for image analysis via machine learning. Locally processed? 870 is determined depending on whether the medical or physiological condition determination is done on the same smart phone or local computer which took at least one photo of the target or eye or if the analysis is done remotely in what this imaging system refers to as the cloud. The cloud comprises at least one computer remotely located from the smart phone or device taking the photos of a target or eye to be used for analysis. If the analysis is to be done on the smart phone or device taking at least one photograph the next step is to process image with ML 871, where ML is machine learning, followed by save image(s), append results with confidence level 872. These results include any metadata associated with the brand of phone including but not limited to type of camera, resolution, and focal length to name a few. Next, encrypt image(s) 873, upload to cloud 874. and update local database with image(s), append results with confidence level 890.

As a rule, any medical and personal data is always encrypted before being uploaded to the cloud or any other machine via the internet. Any data processing on any smart device or computer can be performed on unencrypted files, but any passing to other computers and databases will be encrypted. If further processing on another smart device or at least one computer is performed the data will be decrypted for computational purposes but results will be encrypted to protect all personal information that resides on any machine or database.

If image data is not to be locally processed where the answer to locally processed? 870, then encrypt image(s) 880, upload to cloud 881, and when on at least one other smart device or computer decrypt image(s) 882, and process image with ML 883. Once again, encrypt image(s) 884 and update local database with image(s), append results with confidence level 890.

FIG. 18 depicts the downloading and launching of the user application, identification of the smart device parameters and acquisition of meta data, the registration process, and training on the use of a smart device to take photographs useful for diagnostic purposes. The first step is to download user application 1001 followed by launch user application 1002. The user application then will read device settings 1003 and commence user registration 1004. The user application will then send registration and device information to Server(s) 1005 and save registration to server 1006. Next, is device or phone model found within server? 1007. This encompasses the device data base or knowledge base contained within the platform. If yes then download device-specific configuration from server 1008. If no, then download default/best fit configuration from server 1009 and set unknown configuration flag 1010. Whether device-specific or default/best fit, save configuration data 1011. It is now time to begin training process 1012, play training video 1013 and display instructions on screen 1014.

The first step is to position camera with screen facing user 1015. Then perform X axis training 1016, perform Y axis training 1017, and perform Z axis training 1018. Next, rotate camera with screen facing away from user 1019 and perform X axis training 1020, perform Y axis training 1021, and perform Z axis training 1022. Then set feedback attributes 1023 which has optimized the sounds to guide the user to position the phone properly for taking photographs.

Now the user can run sample test on target 1024 to see if the process works. If the answer to is user application satisfied with results? 1025 is yes, the user application is satisfied with the results then save configuration data 1026 and upload data to cloud 1027.

If the answer to is user application satisfied with results? 1025 is no, the user application is not satisfied with results and returns to display instructions on screen 1014 and repeats the training process with the phone facing towards and away from the user and continue until the answer to is user application satisfied with results? 1025 is yes, in which case the user application will save configuration data 1026 and upload data to cloud 1027.

The imaging system further comprises the ability to take or capture videos or at least one still image of a person or a portion of a person and perform user impairment diagnostic analysis to determine at least one of: user response time to an optical or audio or optical and audio stimulus, ability to follow a target image displayed on a visual display, visually determined or measure and rate motor coordination based on user image parameter response, determine or measure and rate motor coordination based on acceleration data acquired by at least one accelerometer or at least one gyroscope or at least one accelerometer and gyroscope or at least sensor available on a smartphone, measure user pupil dilation, measure at least one user pupil movement and tracking, measure and quantify erratic movement of at least one user eyeball, measure and quantify user facial expressions parameters, measure and quantify user gait, measure, quantify, and assess other biometric parameters that can be sensed by a smartphone, and the ability to measure user response to at least one said stimulus sound in a prescribed manner.

This can involve generating at least one target image on the display screen of a smartphone and taking or capturing a video of the response of the eye to follow the target image or assess attributes of the target image. The stimulus target images or sounds or images and sounds can also be used as the target to guide a user in a prescribed manner to move at least one of head movement, at least one eye movement, at least one hand movement, at least one finger movement, walking, standing, turning, or any combination thereof.

Audio tones or verbal commands or instructions in at least one language or audio tones and verbal commands and instructions or audio tones and verbal commands can also be used to guide a user to respond to stimulus such as the generation of at least one frequency, at least one changing frequency, at least one frequency of at least one duration in time, at least one frequency of at least one duration in time followed by a dwell of at least one other duration of time followed by at least one frequency of at least one duration in time, or any combination thereof.

For instance, a baseline for sobriety or non-impairment can be determined during a training session or a series of training sessions and the same or similar test can be initiated to test the response at another time to determine whether the same user is impaired or under the influence of a substance such as including but not limited to at least one of alcohol, cannabis, opiates, fentanyl, amphetamines, barbiturates, psychedelic or psychoactive drugs, or any medication that can affect physical or mental performance or both. Impairment can also result from no ingestion of any intoxicant but could result from a stroke, heart attack or cardiac events with physical performance ramifications such as head or other physical trauma, asthma, illness or disease of any kind, motion sickness, altitude sickness, Parkinson's disease, essential tremor, migraines, diabetes, neuropathy, paralysis, dehydration, heat stroke, sleep deprivation, exhaustion, hunger, sleepiness, pregnancy, or other naturally occurring condition or a combination thereof that can impair a person and should be flagged to prevent operation of machinery, hand and power tools, motorized vehicles of all kinds and all purposes, boats, submarines, winged aircraft, helicopters, drones, recreational vehicles, jet skis, snowmobiles, or engaging in recreational activities where impairment can be dangerous to oneself or others such as professional or amateur sports including but not limited to baseball, football, ice hockey, basketball, soccer, polo, downhill skiing, cross country skiing, snowboarding, water skiing, surfing, swimming, skydiving, hang gliding, windsurfing, scuba diving using any diving technology, rock climbing, mountain climbing, hiking, caving of any type, rappelling and ascending or ropework, and rescue activities to name a few. Many occupations require top mental and physical performance such as air traffic controllers, pilots, navigators, train engineers, bus drivers, medical professionals such as doctors, surgeons, anesthesiologists, pharmacists, nurses, nursing home and assisted living facility workers, dentists, dental technicians, EMS workers, lifeguards, first responders, hospital workers, X-ray technicians, MRI and CAT scan operators, veterinarians, dispatchers, amusement park ride operators, administrators, food preparation personnel, actors and performers, repair people, ranchers, farmers, fishermen, operators of power plants of all technologies, oil rig personnel, baggage handlers, crane operators, construction workers, demolition workers, judges, law enforcement professionals including police and civilian employees, and in all occupations within all branches of the military to name a few—in short any occupation where an avoidable judgement error can result in loss of life, injury, or property damage, all of which can represent billions of dollars in insurance payouts after potentially expensive litigation.

In one embodiment, the impairment detection subsystem measures saccadic eye movement parameters. A saccade is a rapid, ballistic eye movement from one fixation point to another. When a target stimulus appears or moves on the display of the smartphone, the camera records the user's eye response. The latency of the saccadic response, measured as the time elapsed between target onset and initiation of eye movement, is a clinically established biomarker for impairment from alcohol, cannabis, benzodiazepines, opiates, sleep deprivation, and certain neurological conditions including early Parkinson's disease. In a non-impaired baseline state, saccadic latency is typically in the range of 150 to 250 milliseconds. Deviations of greater than a threshold from the user's established baseline are flagged as potential impairment indicators. The imaging system measures saccadic latency using the smartphone camera operating in video mode at a frame rate sufficient to resolve the onset of eye movement, which for standard smartphones is at least 30 frames per second and for high-speed video modes may be 120, 240, or 960 frames per second.

In another embodiment, the impairment detection subsystem measures smooth pursuit tracking accuracy. Smooth pursuit is the eye's ability to follow a slowly moving target with a continuous, smooth tracking motion. The imaging system displays a target that moves continuously across the smartphone display in a predetermined trajectory and records the user's eye movement via the smartphone camera. Impairment from alcohol, cannabis, stimulants, sedatives, or fatigue degrades smooth pursuit accuracy, producing characteristic tracking errors including: saccadic intrusions, in which the smooth tracking motion is interrupted by small rapid jumps; gain reduction, in which the eye velocity is lower than the target velocity; and increased phase error. The imaging system computes a smooth pursuit gain coefficient as the ratio of measured eye velocity to target velocity, and quantifies saccadic intrusion frequency and amplitude, comparing these metrics against the user's established baseline.

In another embodiment, the impairment detection subsystem quantifies nystagmus. Nystagmus is an involuntary, repetitive oscillatory movement of one or both eyes. Horizontal gaze nystagmus (HGN), the involuntary oscillation of the eyes when held at lateral gaze, is a primary field sobriety indicator for alcohol impairment and is legally admissible evidence of intoxication in many jurisdictions. The imaging system detects HGN by instructing the user to follow a target moving to the extreme left or right of the display and analyzing the video of the eye for the presence of involuntary oscillation at or near the angle of maximum deviation. The smartphone camera records the eye at close range, and image processing algorithms track the position of the pupil centroid at video frame rate to detect oscillatory displacement. The imaging system additionally measures the angle of onset of nystagmus, the frequency and amplitude of oscillatory motion, and whether distinct nystagmus before 45 degrees of lateral deviation is present.

In another embodiment, the impairment detection subsystem measures the pupillary light reflex (PLR). The imaging system presents a light stimulus of controlled intensity and duration via the smartphone display and records the pupil's response with the front-facing camera. Metrics extracted from the PLR include: latency from stimulus onset to initiation of constriction; maximum constriction amplitude; constriction velocity; re-dilation velocity after the stimulus is removed; and the pupil escape index, which measures re-dilation during sustained illumination. Stimulants such as amphetamines and cocaine cause pupil dilation and blunted constriction response. Opiates and sedatives cause pupil constriction and reduced PLR amplitude. By comparing the measured PLR metrics against the user's established baseline and population norms, the imaging system identifies patterns of drug-class-specific impairment with a specificity not achievable from a single static pupil diameter measurement.

In another embodiment, the impairment detection subsystem measures motor coordination using the smartphone's inertial sensors. The imaging system employs at least one accelerometer and at least one gyroscope integrated within the smartphone to measure fine motor performance of a user executing a guided physical task. In one configuration, the user holds the smartphone in a specified position while the accelerometer records involuntary postural tremor and sway. Elevated tremor frequency and amplitude are associated with alcohol intoxication, benzodiazepine use, Parkinson's disease, essential tremor, and fatigue. In another configuration, the user performs a finger-to-target task on the smartphone display, and the precision, path length, and reaction time of the touch are recorded. The combination of eye-based and motor-based biomarkers provides multi-modal impairment assessment with higher specificity and sensitivity than either modality alone.

In one embodiment a dot or target of any display type can appear on a display screen of a smartphone or smart device and a user can respond to the movement of the target and the response time can be measured. Pupil response can be measured as a function of changing light conditions. The target can do any of the following including but not limited to move in any direction, appear in one location, disappear and reappear in another location, blink on and off, change color or intensity, change shape, display several similar shapes and require identification of a different shape or attribute of at least one shape. The target can also change focus or require some user-action to maintain the focus of an image by moving a smart device closer or farther away from the user.

In another embodiment the smartphone can send a signal to at least one external visual display device which can receive the signal sent from the smartphone and can change the visual display that the user can respond to, and the smartphone camera can watch the user response. One embodiment of said external display can be a device with at least one LED or other light sources located on at least one arms or telescoping arm that when extended can increase the angular viewing field of a user to a greater angle than can be displayed on a smartphone screen. With known send and receive propagation times between the smartphone and at least one external visual display device the user response time can be accurately ascertained. One scenario for example only would be a central receiving device with a multiplicity of telescoping arms extending the visual field. Commands could be sent to turn on one light, then turn that light off and turn another light on and measure the user response. Different test patterns can be executed to measure user response. Communication can occur via Bluetooth, Wi-Fi, radio, acoustically, or can be via attached wire, and communication can be unidirectional or duplex.

In one embodiment the imaging system is a software application that transforms a smartphone containing at least one camera and a display with a display central axis into a medical imaging and diagnostic device capable of photographing a target or eye or other body part at both the correct focal length and centered about said display central axis.

It comprises the ability to take at least one positioning photograph or video of a target or eye or other body part to establish positioning information to generate at least one feedback sound to guide a user to move the smartphone to a position centered about the display central axis on the smartphone and at the correct focal length or distance.

This involves the ability to determine the horizontal displacement along a horizontal axis and the vertical displacement along a vertical axis between the target or eye central axis and the display central axis. This further involves the ability to establish a major axis and minor axis of an image of the target or eye or a portion thereof to establish an angle between at least one camera and the target or eye central axis. It should be noted that when a circle is viewed off axis it appears elliptical and the ellipse can be analyzed to establish a major and minor axis, and thus from this information alone the horizontal and vertical components of the major and minor axes can be used to produce feedback sounds to guide a user to position the smartphone normal to the pupil.

To take at least one photograph the angular alignment guidance subsystem is first engaged wherein at least one feedback sound comprises the generation of at least one audio sound type possessing at least one audio parameter that is proportional to the displacement from display central axis and rotation about at least one axis. This also involves the ability to turn off at least one audio sound type when the camera is aligned normal to the central axis, thus constituting angular alignment. After angular alignment is achieved the focal length guidance subsystem is engaged wherein at least one feedback sound comprises the generation of a focal audio sound type possessing at least one focal audio parameter. The focal audio sound type is produced only during the condition constituting angular alignment, and the focal audio parameter is proportional to the absolute value of the difference between the distance between the camera correct focal length and the target or eye, and the focal audio parameter further produces a peak condition indicating the correct focal length, which then triggers or is coincident with the taking of at least one diagnostic photograph until a preset number of diagnostic photographs have been taken. In the event that angular alignment is lost, the focal audio sound type is shut off, thus reinitiating the angular alignment guidance subsystem which continues until angular alignment is achieved, thus causing the re-initiation of the focal length guidance subsystem, and when the peak condition is achieved, this triggers or is coincident with the taking of at least one diagnostic photograph until a preset number of diagnostic photographs has been taken.

This involves the ability to determine that at least one diagnostic photograph of the target or eye or other body part or a portion thereof is in focus, which is followed by the ability to save, encrypt, and store at least one diagnostic photograph or at least one composite diagnostic photograph with identifying data.

For an angular alignment guidance subsystem with two sounds at least one feedback sound comprises at least one audio sound type possessing at least one audio parameter that is proportional to the displacement from the display central axis and rotation about at least one axis. More specifically at least one feedback sound is a first audio sound type possessing at least one first audio parameter that is proportional to both horizontal displacement from the display central axis and rotation about a vertical axis and a second audio sound type possessing at least one second audio parameter that is proportional to both the vertical displacement from the display central axis and rotation about a horizontal axis. The focal audio sound type possesses at least one focal audio parameter.

For an angular alignment guidance subsystem with one sound at least one feedback sound comprises at least one audio sound type possessing at least one audio parameter that is proportional to displacement from the display central axis and rotation about at least one axis and is a conical audio sound type possessing at least one conical audio parameter that is proportional to both the horizontal displacement from the display central axis and rotation about a vertical axis and is proportional to the vertical displacement from the display central axis and rotation about a horizontal axis. The focal audio sound type possessing at least one focal audio parameter.

The ability to turn off at least one audio sound type when the camera is aligned normal to the central axis is the ability to turn off the first audio sound type when the camera is aligned normal to the central axis and the ability to turn off the second audio sound type when the camera is aligned normal to the central axis, both conditions together thus constituting angular alignment.

The ability to turn off at least one audio sound type when the camera is aligned normal to the central axis is the ability to turn off the conical audio sound type when the camera is aligned normal to the central axis, thus constituting angular alignment.

The first audio sound type can comprise a continuously sounding first musical note or fundamental frequency that decreases or increases as the phone moves closer to alignment in a first sense of translation and rotation and the second audio sound type can comprise a pulse train that consists of a second musical note or fundamental frequency of a fixed time duration followed by a variable off time. The fixed time duration plus the variable off time defines the pulse repetition rate which increases or decreases as the phone moves closer to alignment in a second sense of translation and rotation.

The conical audio sound type can comprise either a continuously sounding first musical note or fundamental frequency that decreases or increases as the phone moves closer to alignment or a pulse train that consists of a second musical note or fundamental frequency of a fixed time duration followed by a variable off time. The fixed time duration plus the variable off time defines the pulse repetition rate which increases or decreases as the phone moves closer to alignment.

The continuously sounding first musical note or fundamental frequency that decreases or increases as the phone moves closer to alignment in a first sense of translation and rotation can be a lowest musical note of fundamental frequency that is 400 HZ+/−100 HZ and highest musical note of fundamental frequency that is 2,000 HZ+/−500 HZ, and the pulse train consists of a second musical note or fundamental frequency that is 1,000+/−500 HZ for a fixed time duration of 200 milliseconds+/−100 milliseconds followed by a variable off time duration ranging between 100 milliseconds+/−50 milliseconds and 888 milliseconds+/−150 milliseconds. The fixed time duration plus the variable off time defines the pulse repetition rate which increases or decreases as the phone moves closer to alignment in a second sense of translation and rotation.

The conical audio sound type can comprise a continuously sounding first musical note or fundamental frequency that decreases or increases as the phone moves closer to alignment, and the first musical note or fundamental frequency can be a lowest musical note or fundamental frequency that is 400 HZ+/−100 HZ and highest musical note or fundamental frequency that is 2,000 HZ+/−500 HZ.

The conical audio sound type can also comprise a pulse train that consists of a second musical note or fundamental frequency that is 1,000+/−500 HZ for a fixed time duration of 200 milliseconds+/−100 milliseconds followed by a variable off time duration ranging between 100 milliseconds+/−50 milliseconds and 888 milliseconds+/−150 milliseconds. The fixed time duration plus the variable off time defines the pulse repetition rate which increases or decreases as the phone moves closer to alignment in a second sense of translation and rotation.

The focal audio sound type possessing at least one focal audio parameter can be a continuous focal note or fundamental frequency that increases and is proportional to the absolute value of the difference between the distance between the camera correct focal length and the target or eye. The peak condition indicates the correct focal length is a maximum note or fundamental frequency.

The focal audio sound type can also be a continuous focal note or fundamental frequency that decreases and is proportional to the absolute value of the difference between the distance between the camera the correct focal length and the target or eye. The peak condition indicates the correct focal length is a minimum note or fundamental frequency.

The continuous focal note or fundamental frequency can range between 1,000 HZ+/−100 HZ when the camera is too close or too far from the target or eye and 1,300 HZ+/−100 HZ can indicate the peak condition at the correct focal length, thus executing either a glissando or portamento between when the camera is too close or too far and when the peak condition is achieved.

The continuous focal note or fundamental frequency can range between 1,300 HZ+/−100 HZ when the camera is too close or too far from the target or eye and 1,000 HZ+/−100 HZ, thus indicating the peak condition at the correct focal length, executing either a glissando or portamento between when the camera is too close or too far and when the peak condition is achieved.

In the context of this imaging system at least one composite photograph is composed of only the in-focus portions of at least two diagnostic photographs and comprises a simple two-component composite photograph that comprises a first in-focus portion of a first diagnostic photograph and a second in-focus portion of a second diagnostic photograph.

The first in-focus portion can be saved and separated from the first out of focus portion by a first focus locus boundary. And the second in-focus portion can be saved and separated from the second out of focus portion by a second focus locus boundary. The first in-focus portion overlaps the second in-focus portion, and a new first-second focus locus edge is located between the first focus locus boundary and the second focus locus boundary, and the first-second focus locus edge is now the new edge of the first in-focus portion of area A1 and the second in-focus portion of area B1 to produce a contiguous first-second composite photograph with no blank or overlapped pixels.

At least one composite photograph can also be a simple three-component composite photograph that comprises a first in-focus portion of a first diagnostic photograph and a second in-focus portion of a second diagnostic photograph and a third in-focus portion of a third diagnostic photograph. The first in-focus portion can be saved and separated from the first out of focus portion by a first focus locus boundary. The said second in-focus portion can be saved and separated from the second out of focus portion by a second focus locus boundary. And the third in-focus portion can be saved and separated from the third out of focus portion by a third focus locus boundary.

The first in-focus portion overlaps the second in-focus portion, and a new first-second focus locus edge is located between the first focus locus boundary and the second focus locus boundary, and the first-second focus locus edge is the new edge of the first in-focus portion of area A2 and the second in-focus portion of area B2 and the second in-focus portion overlaps the third in-focus portion, and a new second-third focus locus edge is located between the second focus locus boundary and the third focus locus boundary, and the second-third focus locus edge is the new edge of the second in-focus portion of area B2 and the third in-focus portion of area C2 to produce a contiguous first-second-third composite photograph with no blank or overlapped pixels.

At least one composite photograph can also be a complex three-component composite photograph comprises a first in-focus portion of a first diagnostic photograph and a second in-focus portion of a second diagnostic photograph a third in-focus portion of a third diagnostic photograph. The first in-focus portion can be saved and separated from the first out of focus portion by a first focus locus boundary. The second in-focus portion can be saved and separated from the second out of focus portion by a second focus locus boundary. The third in-focus portion can be saved and separated from the third out of focus portion by a third focus locus boundary.

The first in-focus portion overlaps the second in-focus portion, and a new first-second focus locus edge is located between the first focus locus boundary and the second focus locus boundary, and the first-second focus locus edge is the new edge of the first in-focus portion of area A3 and the second in-focus portion of area B3 and the first in-focus portion overlaps the third in-focus portion, and a new first-third focus locus edge is located between the first focus locus boundary and the third focus locus boundary, and the first-third focus locus edge is the new edge of the first in-focus portion of area A3 and the third in-focus portion of area C3 and the second in-focus portion overlaps the third in-focus portion, and a new second-third focus locus edge is located between the second focus locus boundary and the third focus locus boundary, and the second-third focus locus edge is the new edge of the second in-focus portion of area B3 and the third in-focus portion of area C3 to produce a contiguous first-second-third composite photograph with no blank or overlapped pixels.

The simple two-component composite photograph can comprise a two-component color and intensity equalization wherein the first in-focus portion of area A1 or more generally area Ag of the simple two-component composite photograph is the larger or equal area with an average color and intensity value at the first-second focus locus edge of Aab and the second in-focus portion of area B1 or more generally area Bg of the simple two-component composite photograph is the smaller or equal area with an average color and intensity value at the first-second focus locus edge of Bba, and the first in-focus portion of area A1 or more generally area Ag and the second in-focus portion of area B1 or more generally area Bg are color and intensity corrected at the first-second focus locus edge to produce no discontinuity in color and intensity at the first-second focus locus edge. The composite photograph has an area of A1+B1, or more generally Ag+Bg and the average color and intensity differential is Aab−Bba for the first in-focus portion and Bba−Aab for the second in-focus portion. All pixels of the first in-focus portion are altered proportional to an intermediate value according to the relation: new color and intensity in the first in-focus portion=Aabnew and Aabnew=Aab+(B1/(A1+B1))×(Aab−Bba), or more generally: new color and intensity in the first in-focus portion Aabnew and Aabnew=Aab+(Bg/(Ag+Bg))×(Aab−Bba), and this value or a percentage thereof is added to all pixel values within the first in-focus portion, and all pixels of the second in-focus portion are altered proportional to an intermediate value according to the relation: new color and intensity in the second in-focus portion=Bbanew and Bbanew=Bba+(A1/(A1+B1))×(Bba−Aab), or more generally: new color and intensity in the second in-focus portion=Bbanew and Bbanew=Bba+(Ag/(Ag+Bg))×(Bba−Aab), and this value or a percentage thereof is added to all pixel values within the second in-focus portion. The process of altering all pixels within the first in-focus portion and all pixels within the second in-focus portion constitutes the two-component color and intensity equalization to produce a color and intensity equalized composite photograph.

The simple three-component composite photograph is formed using a simple three-component color and intensity equalization which comprises a first two-component color and intensity equalization followed by a second two-component color and intensity equalization. The first in-focus portion of area Ag of the simple three-component composite photograph has an average color and intensity value at the first-second focus locus edge of Aab, and the second in-focus portion of area Bg of the simple three-component composite photograph has an average color and intensity value at the first-second focus locus edge of Bba and the second in-focus portion of area Bg of the simple three-component composite photograph has an average color and intensity value at the second-third focus locus edge of Bbc, and the third in-focus portion of area C or more generally Cg of the simple three-component composite photograph has an average color and intensity value at the second-third focus locus edge of Ccb.

The first two-component color and intensity equalization employs the generalized kernel equations: new color and intensity within in-focus portion of area Xn1=Xxy+(Y/(X+Y))×(Xxy−Yyx), or more specifically Aabn1=Aab+(Bg/(Ag+Bg))×(Aab−Bba), and this value or a percentage thereof is added to all pixel values within the first in-focus portion. The new color and intensity within in-focus portion of area Yn1=Yyx+(X/(X+Y))×(Yyx−Xxy), or more specifically Bban1=Bba+(Ag/(Ag+Bg))×(Bba−Aab), and this value or a percentage thereof is added to all pixel values within the second in-focus portion, where Xxy=Aab, Yyx=Bba, X=Ag, and Y=Bg are substituted into the generalized kernel equations to produce Xn1=Aabn1=Yn1=Bban1, and thus the first in-focus portion of area Ag and the second in-focus portion of area Bg are color and intensity corrected at the first-second focus locus edge to produce no discontinuity in color and intensity at the first-second focus locus edge, and this produces the new corresponding values of Aabn1 and Bbcn1.

The second two-component color and intensity equalization employs the generalized kernel equations: new color and intensity within in-focus portion of area Xn2=Xxy+(Y/(X+Y))×(Xxy−Yyx), or more specifically Bbcn2=Bbcn1+ (Cg/(Ag+Bg+Cg))×(Bbcn1−Ccb), and this value or a percentage thereof is added to all pixel values within the first in-focus portion and the second in-focus portion, and new color and intensity within in-focus portion of area Yn2=Yyx+(X/(X+Y))×(Yyx−Xxy), or more specifically Ccbn2=Ccb+((Ag+Bg)/(Ag+Bg+Cg))×(Ccb−Bbc), and this value or a percentage thereof is added to all pixel values within the third in-focus portion, where Xxy=Bbcn1, Yyx=Ccbn1, X=Ag+Bg, and Y=Cg are substituted into the generalized kernel equations to produce Xn2=Bbcn2=Yn1=Ccbn2, and thus the second in-focus portion of area Bg and the third in-focus portion of area Cg are color and intensity corrected at the second-third focus locus edge to produce no discontinuity in color and intensity at the second-third focus locus edge, and this produces a new corresponding value of Bban2 which replaces Bban1 and Aabn2 which replaces Aabn1, and Aabn2 is equal to Bban2.

The process of altering all pixels within the first in-focus portion and all pixels within the second in-focus portion and all pixels within the third in-focus portion constituting the simple three-component color and intensity equalization to produce a color and intensity equalized composite photograph.

The complex three-component composite photograph is formed using a complex three-component color and intensity equalization which comprises a first two-component color and intensity equalization followed by a second two-component color and intensity equalization followed by a third two-component color and intensity equalization followed by a fourth two-component color and intensity equalization, followed by at least one repetition of the above two-component color and intensity equalizations until there is no color and intensity discontinuity between any two of the component in-focus diagnostic photographs.

The first in-focus portion of area Ag of the complex three-component composite photograph has an average color and intensity value at the first-second focus locus edge of Aab, and the first in-focus portion of area Ag of the complex three-component composite photograph has an average color and intensity value at the first-third focus locus edge of Aac. The second in-focus portion of area Bg of the complex three-component composite photograph has an average color and intensity value at the first-second focus locus edge of Bba and the second in-focus portion of area Bg of the complex three-component composite photograph has an average color and intensity value at the second-third focus locus edge of Bbc. The third in-focus portion of area C or more generally Cg of the complex three-component composite photograph has an average color and intensity value at the first-third focus locus edge of Cca and the third in-focus portion of area Cg of the complex three-component composite photograph has an average color and intensity value at the second-third focus locus edge of Ccb.

The first two-component color and intensity equalization employs the generalized kernel equations: new color and intensity within in-focus portion of area Xn1=Xxy+(Y/(X+Y))×(Xxy−Yyx), or more specifically Aabn1=Aab+(Bg/(Ag+Bg))×(Aab−Bba), and this value or a percentage thereof is added to all pixel values within the first in-focus portion, and new color and intensity within in-focus portion of area Yn1=Yyx+(X/(X+Y))×(Yyx−Xxy), or more specifically Bban1=Bba+(Ag/(Ag+Bg))×(Bba−Aab), and this value or a percentage thereof is added to all pixel values within the second in-focus portion, where Xxy=Aab, Yyx=Bba, X=Ag, and Y=Bg are substituted into the generalized kernel equations to produce Xn1=Aabn1=Yn1=Bban1, and thus the first in-focus portion of area Ag and the second in-focus portion of area Bg are color and intensity corrected at the first-second focus locus edge to produce no discontinuity in color and intensity at the first-second focus locus edge, and this produces the new corresponding values of Aabn1, Aacn1, Bban1, Bbcn1, Ccan1, and Ccb1 where Aabn1 is equal to Bban1, Ccan1 is equal to Cca, and Ccbn1=Ccb.

The second two-component color and intensity equalization employs the generalized kernel equations: new color and intensity within in-focus portion of area Xn2=Xxy+(Y/(X+Y))×(Xxy−Yyx), or more specifically ABacn2=Aacn1+(Cg/(Ag+Bg+Cg))×(Aacn1−Ccan1), and this value or a percentage thereof is added to all pixel values within the first in-focus portion and the second in-focus portion, and new color and intensity within in-focus portion of area Yn2=Yyx+(X/(X+Y))×(Yyx−Xxy), or more specifically Ccan2=Ccan1+((Ag+Bg)/(Ag+Bg+Cg))×(Ccan1−Aacn1), and this value or a percentage thereof is added to all pixel values within the third in-focus portion, where Xxy=Aacn1, Yyx=Ccan1, X=Ag+Bg, and Y=Cg are substituted into the generalized kernel equations to produce Xn2=ABacn2=Yn2=Ccan2, and thus the first in-focus portion of area Ag and the third in-focus portion of area Cg are color and intensity corrected at the first-third focus locus edge to produce no discontinuity in color and intensity at the first-third focus locus edge.

This produces a new corresponding value of Bban2 which replaces Bban1 and Aabn2 which replaces Aabn1, and Aabn2 is equal to Bban2, and Bbcn2 replaces Bbcn1, and Ccbn2 replaces Ccbn1.

The third two-component color and intensity equalization employs the generalized kernel equations: new color and intensity within in-focus portion of area Xn3=Xxy+(Y/(X+Y))×(Xxy−Yyx), or more specifically ACcbn3=Ccbn2+(Bg/(Ag+Bg+Cg))×(Ccbn2−Bbcn2), and this value or a percentage thereof is added to all pixel values within the first in-focus portion and the second in-focus portion, and new color and intensity within in-focus portion of area Yn3=Yyx+(X/(X+Y))×(Yyx−Xxy), or more specifically Bbcn3=Ccbn2+((Ag+Cg)/(Ag+Bg+Cg))×(Bbcn2−Ccbn2), and this value or a percentage thereof is added to all pixel values within the third in-focus portion, where Xxy=Ccbn2, Yyx=Bbcn2, X=Ag+Cg, and Y=Bg are substituted into the generalized kernel equations to produce Xn3=ACcbn3=Yn3=Bbcn3, and thus the first in-focus portion of area Ag and the third in-focus portion of area Cg are color and intensity corrected at the second-third focus locus edge to produce no discontinuity in color and intensity at the first-third focus locus edge. This produces a new corresponding value of Ccbn3 which replaces Ccbn2 and Bbcn3 which replaces Bbcn2 and Ccbn3 equals Bbcn3, and Aacn3 replaces Aacn2, and Ccan3 replaces Ccan2, and Aacn3 is equal to Ccan3, and Bban3 replaces Bban2 and Aabn3 replaces Aabn2.

The fourth two-component color and intensity equalization employs the generalized kernel equations: new color and intensity within in-focus portion of area Xn4=Xxy+(Y/(X+Y))×(Xxy−Yyx), or more specifically BCbcn4=Bban3+(Ag/(Ag+Bg+Cg))×(Bban3−Aabn3), and this value or a percentage thereof is added to all pixel values within the second in-focus portion and the third in-focus portion, and new color and intensity within in-focus portion of area Yn4=Yyx+(X/(X+Y))×(Yyx−Xxy), or more specifically Aabn4=Aabn3+((Bg+Cg)/(Ag+Bg+Cg))×(Aabn3−Bban3), and this value or a percentage thereof is added to all pixel values within the first in-focus portion, where Xxy=Bban3, Yyx=Aabn3, X=Bg+Cg, and Y=Ag are substituted into the generalized kernel equations to produce Xn4=BCbcn4=Yn4=Aabn4, and thus the first in-focus portion of area Ag and the second in-focus portion of area Bg are color and intensity corrected at the first-second focus locus edge to produce no discontinuity in color and intensity at the first-second focus locus edge, and this produces a new corresponding value of Ccbn4 which replaces Ccbn3 and Bbcn4 which replaces Bbcn3 and Ccbn4 equals Bbcn4, and Aacn4 replaces Aacn3, and Ccan4 replaces Ccan3, and Bban4 replaces Bban3 and Aabn4 replaces Aabn3 and Bban4 is equal to Aabn4.

The second two-component color and intensity equalization followed by the third two-component color and intensity equalization which is followed by the fourth two-component color and intensity equalization repeats as many times as is necessary until there is no discontinuity in color and intensity at the first-second focus locus edge and at the first-third focus locus edge and at the second-third focus locus edge. The process of altering all pixels within the first in-focus portion and all pixels within the second in-focus portion and all pixels within the third in-focus portion constituting the complex three-component color and intensity equalization to produce a color and intensity equalized composite photograph.

In the tiling of multiple photographs or composite photographs at an irregular or curved boundary or focus locus boundary it is sometimes necessary to create non-rectangular parametric pixels to abut the edge of the irregular or curved boundary. Thus the software application can further comprise the creation of at least one parametric coordinate system comprising a multiplicity of parametric pixels that superimpose upon the rectangular pixel grid comprising a multiplicity of rectangular pixels taken by a digital camera. The intensity and color value of each parametric pixel contained within the set of a multiplicity of parametric pixels is determined by the weighted average of the intensity and color values a subset of the multiplicity of overlapped portions of rectangular pixels intersecting the parametric pixel. At least one mathematical operation can then be performed on and between at least one first subset of a multiplicity of parametric pixels and at least one second subset of a multiplicity of parametric pixels, which is where the intensity and color values at the boundary is blended between at least two parametric pixels. Then the intensity and color values of the rectangular pixels are redefined to become transformed rectangular pixels. The intensity and color value of at least one transformed rectangular pixel is determined by overlapped portions a multiplicity of parametric pixels that superimpose upon the transformed rectangular pixel. The intensity and color value of each transformed rectangular pixel is determined by the weighted average of the intensity and color values of a subset of the multiplicity of overlapped portions of parametric pixels intersecting the rectangular pixel.

The angular alignment guidance subsystem comprises or further comprises the ability to acquire the reflection of a smartphone within the eye to determine the horizontal component of displacement along a horizontal axis and the vertical component of displacement along a vertical axis between the eye central axis and the display central axis, and the horizontal component and the vertical component is used in whole or supplementally in part to create at least one feedback sound to position the smartphone by a user.

The imaging system further comprises the ability to recognize the reflection of a cellphone or smartphone, person, or other recognizable objects and subtract these features or a portion thereof from a photograph or composite photograph of an eye.

When the smartphone is used in the forward-facing camera configuration to photograph another person's eye, the corneal surface of the eye acts as a convex mirror and reflects a visible image of the smartphone itself. This reflection, commonly referred to as a Purkinje image or corneal reflex, appears in the captured image as a bright highlight that can obscure diagnostic features of the iris and pupil. The imaging system comprises a reflection detection and subtraction module that identifies and computationally removes these reflections from the diagnostic image.

In one embodiment, the reflection detection module employs a trained object recognition model to identify the characteristic rectangular or rounded-rectangular shape of a smartphone reflection within the corneal region of the eye image. The smartphone reflection is typically a bright, low-contrast rectangle positioned within the corneal reflex area, often containing recognizable features of the smartphone front face such as the camera aperture, speaker grille, or screen content. The object recognition model is trained on a dataset of labeled eye images containing smartphone reflections of varying sizes, orientations, and brightnesses. Once detected, the spatial extent of the reflection is segmented from the surrounding iris and scleral tissue.

In another embodiment, the reflection subtraction module replaces the segmented reflection region with synthesized image content. The synthesis method interpolates pixel values from the surrounding non-reflected iris and scleral tissue to reconstruct the obscured diagnostic features beneath the reflection. The interpolation can be performed using any method including but not limited to bilinear interpolation, bicubic interpolation, inpainting using partial differential equations, or neural network-based image inpainting trained on paired ocular images with and without reflections.

In a further embodiment, the position and size of the smartphone reflection within the corneal reflex can be used as an additional input to the angular alignment subsystem.

Because the corneal reflex position is a function of the angle between the smartphone camera and the optical axis of the eye, the deviation of the corneal reflex from the center of the cornea provides an independent measurement of the camera's angular misalignment with the target. This measurement can supplement or replace the ellipse-based angular measurement described elsewhere in this disclosure, providing a second independent channel of angular error information that improves the robustness of the audio feedback guidance system.

At least one diagnostic photograph or at least one composite diagnostic photograph is illumination and camera color-corrected to produce at least one illumination and camera color-corrected diagnostic photograph or at least one illumination and camera color-corrected composite diagnostic photograph by employing at least one illumination and camera color-correction transform or transfer function that maps every color and intensity of each pixel of the diagnostic photograph or composite diagnostic photograph into a different color and intensity of each respective pixel to produce an illumination and camera color-corrected diagnostic photograph or illumination and camera color-corrected composite diagnostic photograph. The illumination and camera color-correction process can compensate for the camera's deviation from accurate quantification of color. If the camera is calibrated then this process can compensate for both this effect and the particular spectrum of the light source. The idea is to produce a normalized image that can make more universal the images used for diagnostics.

At least one diagnostic photograph or at least one composite diagnostic photograph or at least one illumination and camera color-corrected diagnostic photograph or at least one illumination and camera color-corrected composite diagnostic photograph is condition-specific color-altered by employing at least one condition-specific color-correction transform or transfer function that maps every color and intensity of each pixel of the diagnostic photograph or the composite diagnostic photograph or the illumination and camera color-corrected diagnostic photograph or the illumination and camera color-corrected composite diagnostic photograph into a different color and intensity of each respective pixel to produce at least one condition-specific color-corrected diagnostic photograph or at least one condition-specific color-corrected composite diagnostic photograph or at least one condition-specific color-corrected illumination and camera color-corrected diagnostic photograph or at least one condition-specific color-corrected illumination and camera color-corrected composite diagnostic photograph. The process of applying condition-specific color-correction is to alter the color and intensity of a photograph or composite photograph to increase the signal to noise ration and improve the diagnostic and discrimination capability of machine learning. Each condition being tested for can have one or more condition-specific color-corrections applied to a photograph or composite photograph.

The machine learning (ML) diagnostic component of the imaging system operates on the fully processed diagnostic image, that is, the composite diagnostic photograph that has been illumination and camera color-corrected and condition-specific color-conditioned. By operating on this normalized and conditioned image rather than a raw photograph, the machine learning classifier receives an input whose color and spatial properties are standardized across devices, lighting environments, and cameras. This normalization is a critical differentiator from prior art approaches that apply machine learning directly to raw camera output, which introduces device-specific and environment-specific variability that degrades classifier performance.

In one embodiment, the machine learning classifier is a supervised binary classifier trained on a first training dataset comprising a plurality of labeled images each possessing the shared characteristic of representing a positive instance of a condition of interest (the condition-positive dataset), and a second training dataset comprising a plurality of labeled images each possessing the shared characteristic of not representing the condition of interest (the condition-negative dataset). The classifier produces a classification output comprising a binary determination of whether the processed diagnostic image belongs to the condition-positive class or the condition-negative class, together with a confidence level representing the posterior probability of class membership. The classifier can be implemented using any machine learning architecture now known or unknown, including but not limited to convolutional neural networks (CNNs), support vector machines (SVMs), gradient boosting classifiers, random forests, vision transformers, or ensemble methods combining any of the above.

In another embodiment, the machine learning classifier is a multi-class classifier capable of simultaneously discriminating among a plurality of conditions or a plurality of levels of a single condition. For example, a multi-class classifier applied to eye images may simultaneously classify an image as belonging to one of: no condition detected, early-stage diabetic retinopathy, moderate-stage diabetic retinopathy, advanced-stage diabetic retinopathy, malaria-associated retinal changes, or conjunctivitis. In such embodiments the classification output comprises a vector of per-class confidence levels. The imaging system can apply multiple condition-specific color-conditioning transforms sequentially or in parallel, each optimized for a different target condition, producing multiple processed diagnostic images from the same composite photograph, each submitted to its respective classifier.

In a further embodiment, the machine learning model executes locally on the same smart device that captured the diagnostic photographs. Local execution provides immediate results, requires no network connectivity, and eliminates latency. This is particularly valuable in data-limited environments, emergency settings, or locations where uploading protected health information is impractical or legally constrained. The smart device processor executes the model using on-device inference frameworks including but not limited to Apple Core ML, Google ML Kit, TensorFlow Lite, PyTorch Mobile, or any equivalent framework now known or unknown that enables inference on mobile hardware.

In another embodiment, the machine learning model is continuously updated using a federated learning architecture in which anonymized and encrypted diagnostic images and their associated ground-truth labels, confirmed by qualified healthcare professionals, are aggregated across a plurality of users and devices to iteratively refine model weights without centralizing raw patient data. The updated model weights are periodically distributed to enrolled devices, improving classifier performance over time. This federated approach enables the accumulation of diverse training data across geographies, demographics, camera hardware types, and lighting conditions, which further reduces the device-specific variability that the color-correction and color-conditioning pipeline was designed to address.

In the context of this imaging system the eye or other body part can be anything that can be photographed and is not limited to the eye or other body part.

The imaging system further comprises a master crop function to reduce the size of the diagnostic photograph or the composite diagnostic photograph to produce a normalized diagnostic photograph or normalized composite diagnostic photograph. The master crop function produces at least one of the following shapes: rectangular, circular, elliptical, or any bounding shape.

At least one transform can be applied to at least one diagnostic photograph or at least one composite diagnostic photograph or at least one illumination and camera color-corrected diagnostic photograph or at least one illumination and camera color-corrected composite diagnostic photograph or at least one condition-specific color-corrected diagnostic photograph or at least one condition-specific color-corrected composite diagnostic photograph or at least one condition-specific color-corrected illumination and camera color-corrected diagnostic photograph or at least one condition-specific color-corrected illumination and camera color-corrected composite diagnostic photograph for use in analysis and diagnostics. At least one transform can be or employs at least one of: an FFT, a discrete Fourier transform, Walsh transform, Hadamard transform, cosine transform, discrete wavelet transform, Laplace transform, convolution integrals and convolution processing, Canny edge detection, Prewitt algorithm for edge detection, Sobel operators for edge detection, or any transform now known or unknown that can be applied to or act upon a two-dimensional array or image or three-dimensional array or n-dimensional array to increase diagnostic discrimination between possessing and not possessing a particular medical condition or classification or impairment.

In another embodiment the imaging system is an application that transforms a smartphone camera into a change detection alarm which comprises setting up smartphone so camera is watching the subject field, followed by initiating video mode. The smartphone then analyzes in real time at least one video frame and detects at least one change of at least one attribute in at least one location within at least one video frame. Having established in advance a user-selectable threshold of at least one change of at least one attribute in at least one location, this is then used as the standard to which the changing video frames are compared. When the threshold of at least one change has been reached or exceeded at least one alarm is generated to alert a user.

A software application that transforms a smartphone containing at least one camera and a visual display with a display central axis into a user impairment diagnostic system comprising the ability to establish a user baseline response, establish at least one user test response, generating at least one stimulus target image or stimulus sound or stimulus target image and stimulus sound, measure the user response, compare user test performance to baseline performance, assess impairment, and send at least one notification.

Establishing a user baseline response comprises taking at least one video or at least one photograph of a user and aligning the smartphone so the user is centered about the display central axis. Then the smartphone generates at least one stimulus target image or stimulus sound or stimulus target image and stimulus sound. The stimulus target image can be displayed on the visual display, and the stimulus sound can be produced by the smartphone speaker.

The smartphone measures the user response to the stimulus that is at least one of: the user response to visually follow at least one stimulus target image, measure and assess motor coordination based on user image parameter response, determine motor coordination based on acceleration data acquired by at least one accelerometer or at least one gyroscope or at least one accelerometer and gyroscope or at least one sensor available on a the smartphone, measure user pupil dilation to at least one brightness change, measure and quantify user facial expression parameters, measure and quantify user gait, measure, quantify, and assess other biometric parameters that can be sensed by a smartphone, the measurement of user response to at least one stimulus sound in a prescribed manner, the user response comprising at least one of: head movement, at least one eye movement, pupil dilation, at least one hand movement, at least one finger movement, walking, standing, turning, or any combination thereof.

The smartphone then generates at least one metric to establish user baseline performance.

Establishing at least one user test response comprises taking at least one video or at least one photograph of a user and aligning the smartphone so the user is centered about the display central axis. Then the smartphone generates at least one stimulus target image or stimulus sound or stimulus target image and stimulus sound. The stimulus target image can be displayed on the visual display, and the stimulus sound can be produced by the smartphone speaker.

The smartphone measures the user response to the stimulus that is at least one of: the user response to visually follow at least one stimulus target image, measure and assess motor coordination based on user image parameter response, determine motor coordination based on acceleration data acquired by at least one accelerometer or at least one gyroscope or at least one accelerometer and gyroscope or at least one sensor available on a the smartphone, measure user pupil dilation to at least one brightness change, measure and quantify user facial expression parameters, measure and quantify user gait, measure, quantify, and assess other biometric parameters that can be sensed by a smartphone, the measurement of user response to at least one stimulus sound in a prescribed manner, the user response comprising at least one of: head movement, at least one eye movement, pupil dilation, at least one hand movement, at least one finger movement, walking, standing, turning, or any combination thereof.

The smartphone then generates at least one metric to establish user test performance.

The smartphone then compares at least one metric of the user test performance with at least one metric of the user baseline performance to establish the probability of user impairment, and associated confidence level of user impairment assessment. The smartphone then stores results in at least one database located on the smartphone or uploads the results to at least one remote database located in at least one remote location or stores results in at least one database located on the smartphone and upload the results to at least one remote database located in at least one remote location.

The smartphone then sends at least one notification.

In another embodiment the imaging system is a portable display device that generates at least two visible outputs comprising a microprocessor and at least one power source. There is also a power management strategy to power down the display device when not in use for at least one period of time to conserve power.

There is also a shell or plate or backplane or housing to contain or mount the electronics. On the back of the display device is located a hook or hole from which it can be hung against a wall or vertical surface and also a fold-out stand to enable it to stand on a horizontal surface.

There are at least two arms that can extend from the shell or plate or backplane or housing, and this extends the distance between the light sources and the corresponding viewing angle by a user. The two or more arms are either fixed or are hinged arms or arms attached by pivots that can rotate or splay radially outward or are telescoping arms that can extend radially outward or a combination of both. The fixed or hinged arms or arms attached by pivots or telescoping arms each contain at least one light source distally located that can be switched on or off by the microprocessor.

The shell or plate or backplane or housing can contain detents that secure or affix at at least two angles the fixed or hinged arms or arms attached by pivots or said telescoping arms. For instance, if hung from a wall, one embodiment can have four LEDs: a south LED at the 6 o'clock position that is attached to the bottom of the housing. A north LED at the 12 o'clock position can be on a telescoping arm. An east LED at the 3 o'clock position can telescope out and click into a position relative to the housing such that the east LED is located at a perpendicular bisector to the halfway point between the south LED and the north LED. And likewise, west LED at the 9 o'clock position can telescope out and click into a position relative to the housing such that the west LED is located at a perpendicular bisector to the halfway point between the south LED and the north LED, thus making both east and west LEDs mirror images of each other about the north-south axis. This configuration would produce the same orientation and distance between the four mentioned LEDs if placed on a horizontal surface or as a tabletop standing unit. If the north telescoping arm were at a different extended distance, the angles and lengths of the east and west LEDs on their respective arms would have to change as well.

In another wall mounted configuration the south LED can also be on a telescoping arm, and if the south and north telescoping arms extend equal distances then the east and west telescoping arms would be perpendicular to the north-south axis.

There is also a receiving subsystem that is at least one of wireless, wired, or acoustically communicated with. The receiving subsystem can receive signals and commands from at least one smartphone and communicate with or is controlled by the microprocessor. In a typical scenario the smartphone would send a command to turn on a light, and the user would follow the lights visually while the smartphone watches the user response and measures the user response time and also quantify any anomalous eye or facial movement in response to the user attempt to track the light. The smartphone can then repeat the process by turning off the light and turning on a different light, and so on. The portable display device increases the angle of the optical stimulus.

The portable display device of embodiment PP communicates with the smartphone via at least one wireless communication protocol selected from the group consisting of Bluetooth, Bluetooth Low Energy (BLE), Wi-Fi (IEEE 802.11), and near-field communication (NFC), and any combination thereof. In one embodiment, the smartphone acts as the central controller, transmitting commands to the portable display device to switch individual light sources on and off in a predetermined sequence. The portable display device receives these commands via its receiving subsystem and executes them with a response latency of less than 50 milliseconds to ensure accurate measurement of user response time. The round-trip communication latency between the smartphone sending a command and the light source changing state is measured and recorded by the smartphone so that it can be subtracted from the user's measured response time to yield an accurate net response latency attributable solely to the user's physiological reaction time.

In one embodiment, the portable display device comprises four light sources arranged symmetrically about a central axis. A first light source is located at the bottom of the device (6 o'clock position), a second light source is located at the top of the device on a telescoping arm (12 o'clock position), a third light source is located on a first lateral arm at a position equidistant between the first and second light sources (3 o'clock position), and a fourth light source is located on a second lateral arm symmetrically opposite the third light source (9 o'clock position). This configuration creates an angular testing field that spans a maximum horizontal angle of at least 60 degrees and a maximum vertical angle of at least 60 degrees as measured from the user's perspective at the intended testing distance of between 30 and 100 centimeters from the device. The extended angular field is specifically designed to test for horizontal gaze nystagmus, which requires lateral gaze at angles beyond the comfortable viewing range of a smartphone screen alone.

In another embodiment, each light source on the portable display device is an LED or an array of LEDs capable of emitting light at a controllable intensity and in at least one selectable color. The ability to vary light intensity enables the device to be used for pupillary light reflex testing, in which a bright flash of known intensity and duration is presented to the user and the pupillary response is recorded by the smartphone camera. The ability to vary color enables the device to present chromatic stimuli for testing color vision or for presenting stimuli with distinct contrast properties for different target populations. Each LED or LED array is individually addressable by the microprocessor, enabling complex stimulus sequences to be programmed and executed with millisecond precision.

In a further embodiment, the portable display device further comprises at least one orientation sensor, including at least one of: an accelerometer, a gyroscope, or a magnetometer, that measures the orientation of the device with respect to gravity and to the user. The orientation data is transmitted to the smartphone via the wireless communication link, enabling the smartphone to compensate for deviations of the device from its intended orientation and to calculate the precise angular position of each light source relative to the user's line of sight. This enables accurate angular calibration of the stimulus presentation and ensures that angular measurements of eye movement are referenced to a known and verified coordinate system rather than an assumed one. The orientation sensor data further enables detection of unintended movement of the device during testing, which can flag test results as potentially unreliable and prompt the user to repeat the test with the device properly stabilized.

In at least one embodiment, the imaging system further comprises a method of transforming a smart device into a medical diagnostic tool comprising the following steps. First the application is downloaded from at least one source such as a website, storage device, or a direct link with a source smart device. Next the user engages in a training session to learn to take a diagnostic worthy photograph by positioning with the aid of audio feedback the smart device to take at least one photograph containing at least one in-focus portion of a target image. The user is now ready to take a diagnostic photograph by positioning with the aid of audio feedback the smart device to take at least one photograph containing at least one in-focus portion of a target image to produce at least one diagnostic photograph. The application then creates at least one in-focus composite diagnostic photograph from the in-focus portions of at least one photograph. The composite diagnostic photograph is then illumination and camera color corrected to compensate for lighting and to create a normalized diagnostic composite photograph. At least one composite diagnostic photograph or illumination and camera color corrected composite diagnostic photograph is then condition-specific color corrected. The condition-specific color corrected composite diagnostic photograph is then cropped. Then at least one other transform can be applied to increase diagnostic signal to noise discrimination. Then the composite diagnostic photograph is compared to at least one database to determine the probability of possessing or not possessing at least one condition or level of substance impairment herein referred to as the result. Encryption or decryption or encryption and decryption is applied before or after any of the above operations depending on where a modification operation is being performed or stored. For instance if the database for diagnostic evaluation is not on the smart device taking the photographs then the composite diagnostic photograph is encrypted before being uploaded, and it will be decrypted at the destination smart device. Next the result is stored in at least one database along with other identifying data and metadata. And finally at least one notification is sent to at least one location.

Aspects of the present specification may also be described as the following embodiments:
AA1. A software application that transforms a smartphone containing at least one camera and a display with a display central axis into a medical imaging device capable of photographing a target or eye or other body part at both the correct focal length and centered about said display central axis comprising: the ability to take at least one positioning photograph or video of said target or eye to establish positioning information to generate at least one feedback sound to guide a user to move said smartphone to a position centered about said display central axis and at the said correct focal length; the ability to determine the horizontal displacement along a horizontal axis and the vertical displacement along a vertical axis between said target or eye central axis and said display central axis; the ability to establish a major axis and minor axis of an image of said target or eye or a portion thereof to establish an angle between said at least one camera and said target or eye central axis; the initiation of an angular alignment guidance subsystem wherein said at least one feedback sound comprises the generation of at least one audio sound type possessing at least one audio parameter that is proportional to displacement from said display central axis and rotation about at least one axis; the ability to turn off said at least one audio sound type when said camera is aligned normal to said central axis, thus constituting angular alignment; the initiation of a focal length guidance subsystem wherein said at least one feedback sound comprises the generation of a focal audio sound type possessing at least one focal audio parameter, said focal audio sound type being produced only during the condition constituting said angular alignment, said focal audio parameter being proportional to the absolute value of the difference between the distance between the camera said correct focal length and said target or eye, and said focal audio parameter further producing a peak condition indicating said correct focal length, which then triggers or is coincident with the taking of at least one diagnostic photograph until a preset number of diagnostic photographs have been taken; the ability to turn off said focal audio sound type in the event that said angular alignment is lost, thus reinitiating said angular alignment guidance subsystem which continues until said angular alignment is achieved, thus causing the re-initiation of said focal length guidance subsystem, and when said peak condition is achieved triggering or is coincident with the taking of at least one diagnostic photograph until said a preset number of diagnostic photographs have been taken; the ability to determine that said at least one diagnostic photograph of said target or eye or other body part or a portion thereof is in focus; the ability to save, encrypt, and store said at least one diagnostic photograph or at least one composite diagnostic photograph with identifying data.
BB1. The software application of embodiment AA1 wherein said at least one feedback sound comprises: at least one audio sound type possessing at least one audio parameter that is proportional to displacement from said display central axis and rotation about at least one axis, more specifically: a first audio sound type possessing at least one first audio parameter that is proportional to both said horizontal displacement from said display central axis and rotation about a vertical axis and a second audio sound type possessing at least one second audio parameter that is proportional to both said vertical displacement from said display central axis and rotation about a horizontal axis; a focal audio sound type possessing at least one focal audio parameter.
BB2. The software application of embodiment AA1 wherein said at least one feedback sound comprises: at least one audio sound type possessing at least one audio parameter that is proportional to displacement from said display central axis and rotation about at least one axis and is a conical audio sound type possessing at least one conical audio parameter that is proportional to both said horizontal displacement from said display central axis and rotation about a vertical axis and is proportional to said vertical displacement from said display central axis and rotation about a horizontal axis; a focal audio sound type possessing at least one focal audio parameter.
CC1. The software application of embodiment BB1 wherein said ability to turn off said at least one audio sound type when said camera is aligned normal to said central axis is the ability to turn off said first audio sound type when said camera is aligned normal to said central axis and the ability to turn off said second audio sound type when said camera is aligned normal to said central axis, both conditions together thus constituting angular alignment.
CC2. The software application of embodiment BB2 wherein said ability to turn off said at least one audio sound type when said camera is aligned normal to said central axis is the ability to turn off said conical audio sound type when said camera is aligned normal to said central axis, thus constituting angular alignment.
HH1. The software application of embodiment BB1 wherein said first audio sound type comprises a continuously sounding first musical note or fundamental frequency that decreases or increases as the phone moves closer to alignment in a first sense of translation and rotation and said second audio sound type comprises a pulse train that consists of a second musical note or fundamental frequency of a fixed time duration followed by a variable off time, said fixed time duration plus said variable off time defines the pulse repetition rate which increases or decreases as the phone moves closer to alignment in a second sense of translation and rotation.
HH2. The software application of embodiment BB2 wherein said conical audio sound type comprises either a continuously sounding first musical note or fundamental frequency that decreases or increases as the phone moves closer to alignment or a pulse train that consists of a second musical note or fundamental frequency of a fixed time duration followed by a variable off time, said fixed time duration plus said variable off time defines the pulse repetition rate which increases or decreases as the phone moves closer to alignment.
JJ1. The software application of embodiment HH1 wherein said continuously sounding first musical note or fundamental frequency that decreases or increases as the phone moves closer to alignment in a first sense of translation and rotation is a lowest musical note of fundamental frequency that is 400 HZ+/−100 HZ and highest musical note of fundamental frequency that is 2,000 HZ+/−500 HZ, and said pulse train consists of a second musical note or fundamental frequency that is 1,000+/−500 HZ for a fixed time duration of 200 milliseconds+/−100 milliseconds followed by a variable off time duration ranging between 100 milliseconds+/−50 milliseconds and 888 milliseconds+/−150 milliseconds, said fixed time duration plus said variable off time defining the pulse repetition rate which increases or decreases as the phone moves closer to alignment in a second sense of translation and rotation.
JJ2. The software application of embodiment HH2 wherein said conical audio sound type comprises either a said continuously sounding first musical note or fundamental frequency that decreases or increases as the phone moves closer to alignment, said first musical note or fundamental frequency is a lowest musical note of fundamental frequency that is 400 HZ+/−100 HZ and highest musical note or fundamental frequency that is 2,000 HZ+/−500 HZ, or said pulse train consists of a second musical note or fundamental frequency that is 1,000+/−500 HZ for a fixed time duration of 200 milliseconds+/−100 milliseconds followed by a variable off time duration ranging between 100 milliseconds+/−50 milliseconds and 888 milliseconds+/−150 milliseconds, said fixed time duration plus said variable off time defining the pulse repetition rate which increases or decreases as the phone moves closer to alignment in a second sense of translation and rotation.
ZZ. The software application of embodiment AA1 wherein said focal audio sound type possessing at least one focal audio parameter is a continuous focal note or fundamental frequency that increases and is proportional to the absolute value of the difference between the distance between the camera said correct focal length and said target or eye, and said peak condition indicating said correct focal length is a maximum note or fundamental frequency or is a continuous focal note or fundamental frequency that decreases and is proportional to the absolute value of the difference between the distance between the camera said correct focal length and said target or eye, and said peak condition indicating said correct focal length is a minimum note or fundamental frequency.
ZZ1. The software application of embodiment ZZ wherein said continuous focal note or fundamental frequency ranges between 1,000 HZ+/−100 HZ when said camera is too close or too far from said target or eye and 1,300 HZ+/−100 HZ, thus indicating said peak condition at said correct focal length, executing either a glissando or portamento between when said camera is too close or too far and when said peak condition is achieved, or said continuous focal note or fundamental frequency ranges between 1,300 HZ+/−100 HZ when said camera is too close or too far from said target or eye and 1,000 HZ+/−100 HZ indicating said peak condition at said correct focal length, executing either a glissando or portamento between when said camera is too close or too far and when said peak condition is achieved.
FF. The software application of embodiment AA1 wherein said at least one composite photograph is composed of only the in-focus portions of at least two diagnostic photographs and comprises: a simple two-component composite photograph comprising: a first in-focus portion of a first diagnostic photograph, said first in-focus portion being saved and separated from the first out of focus portion by a first focus locus boundary; a second in-focus portion of a second diagnostic photograph, said second in-focus portion being saved and separated from the second out of focus portion by a second focus locus boundary; said first in-focus portion overlapping said second in-focus portion, and a new first-second focus locus edge being located between said first focus locus boundary and said second focus locus boundary, and said first-second focus locus edge being the new edge of said first in-focus portion of area A1 and said second in-focus portion of area B1 to produce a contiguous first-second said composite photograph with no blank or overlapped pixels; or a simple three-component composite photograph comprising: a first in-focus portion of a first diagnostic photograph, said first in-focus portion being saved and separated from the first out of focus portion by a first focus locus boundary; a second in-focus portion of a second diagnostic photograph, said second in-focus portion being saved and separated from the second out of focus portion by a second focus locus boundary; a third in-focus portion of a third diagnostic photograph, said third in-focus portion being saved and separated from the third out of focus portion by a third focus locus boundary; said first in-focus portion overlapping said second in-focus portion, and a new first-second focus locus edge being located between said first focus locus boundary and said second focus locus boundary, and said first-second focus locus edge being the new edge of said first in-focus portion of area A2 and said second in-focus portion of area B2 and said second in-focus portion overlapping said third in-focus portion, and a new second-third focus locus edge being located between said second focus locus boundary and said third focus locus boundary, and said second-third focus locus edge being the new edge of said second in-focus portion of said area B2 and said third in-focus portion of area C2 to produce a contiguous first-second-third said composite photograph with no blank or overlapped pixels; or a complex three-component composite photograph comprising: a first in-focus portion of a first diagnostic photograph, said first in-focus portion being saved and separated from the first out of focus portion by a first focus locus boundary; a second in-focus portion of a second diagnostic photograph, said second in-focus portion being saved and separated from the second out of focus portion by a second focus locus boundary; a third in-focus portion of a third diagnostic photograph, said third in-focus portion being saved and separated from the third out of focus portion by a third focus locus boundary; said first in-focus portion overlapping said second in-focus portion, and a new first-second focus locus edge being located between said first focus locus boundary and said second focus locus boundary, and said first-second focus locus edge being the new edge of said first in-focus portion of area A3 and said second in-focus portion of area B3 and said first in-focus portion overlapping said third in-focus portion, and a new first-third focus locus edge being located between said first focus locus boundary and said third focus locus boundary, and said first-third focus locus edge being the new edge of said first in-focus portion of said area A3 and said third in-focus portion of area C3 and said second in-focus portion overlapping said third in-focus portion, and a new second-third focus locus edge being located between said second focus locus boundary and said third focus locus boundary, and said second-third focus locus edge being the new edge of said second in-focus portion of said area B3 and said third in-focus portion of said area C3 to produce a contiguous first-second-third said composite photograph with no blank or overlapped pixels.
FF1. The software application of embodiment FF wherein said simple two-component composite photograph comprises a two-component color and intensity equalization wherein said first in-focus portion of area A1 or more generally area Ag of said simple two-component composite photograph is the larger or equal area with an average color and intensity value at said first-second focus locus edge of Aab and said second in-focus portion of area B1 or more generally area Bg of said simple two-component composite photograph is the smaller or equal area with an average color and intensity value at said first-second focus locus edge of Bba, and said first in-focus portion of area A1 or more generally area Ag and said second in-focus portion of area B1 or more generally area Bg are color and intensity corrected at said first-second focus locus edge to produce no discontinuity in color and intensity at said first-second focus locus edge, said composite photograph having an area of A1+B1, or more generally Ag+Bg and the average color and intensity differential is Aab−Bba for said first in-focus portion and Bba−Aab for said second in-focus portion, and all pixels of said first in-focus portion are altered proportional to an intermediate value according to the relation: new color and intensity in said first in-focus portion=Aabnew=Aab+ (B1/(A1+B1))×(Aab−Bba), or more generally: new color and intensity in said first in-focus portion Aabnew=Aab+ (Bg/(Ag+Bg))×(Aab−Bba), and this value or a percentage thereof is added to all pixel values within said first in-focus portion, and all pixels of said second in-focus portion are altered proportional to an intermediate value according to the relation: new color and intensity in said second in-focus portion=Bbanew=Bba+(A1/(A1+B1))×(Bba−Aab), or more generally: new color and intensity in said second in-focus portion=Bbanew=Bba+(Ag/(Ag+Bg))×(Bba−Aab), and this value or a percentage thereof is added to all pixel values within said second in-focus portion, the process of altering all pixels within said first in-focus portion and all pixels within said second in-focus portion constituting said two-component color and intensity equalization to produce a color and intensity equalized said composite photograph.
FF2. The software application of embodiment FF1 wherein said simple three-component composite photograph is formed using a simple three-component color and intensity equalization which comprises a first two-component color and intensity equalization followed by a second two-component color and intensity equalization, said first in-focus portion of said area Ag of said simple three-component composite photograph has an average color and intensity value at said first-second focus locus edge of Aab, and said second in-focus portion of said area Bg of said simple three-component composite photograph has an average color and intensity value at said first-second focus locus edge of Bba and said second in-focus portion of said area Bg of said simple three-component composite photograph has an average color and intensity value at said second-third focus locus edge of Bbc, and said third in-focus portion of area C or more generally Cg of said simple three-component composite photograph has an average color and intensity value at said second-third focus locus edge of Ccb; said first two-component color and intensity equalization employing the generalized kernel equations: new color and intensity within in-focus portion of area Xn1=Xxy+(Y/(X+Y))×(Xxy−Yyx), or more specifically Aabn1=Aab+(Bg/(Ag+Bg))×(Aab−Bba), and this value or a percentage thereof is added to all pixel values within said first in-focus portion, and new color and intensity within in-focus portion of area Yn1=Yyx+(X/(X+Y))×(Yyx−Xxy), or more specifically Bban1=Bba+(Ag/(Ag+Bg))×(Bba−Aab), and this value or a percentage thereof is added to all pixel values within said second in-focus portion, where Xxy=Aab, Yyx=Bba, X=Ag, and Y=Bg are substituted into the generalized kernel equations to produce said Xn1=Aabn1=Yn1=Bban1, and thus said first in-focus portion of said area Ag and said second in-focus portion of said area Bg are color and intensity corrected at said first-second focus locus edge to produce no discontinuity in color and intensity at said first-second focus locus edge, and this produces the new corresponding values of Aabn1 and Bbcn1; said second two-component color and intensity equalization employs the generalized kernel equations: new color and intensity within in-focus portion of area Xn2=Xxy+(Y/(X+Y))×(Xxy−Yyx), or more specifically Bbcn2=Bbcn1+(Cg/(Ag+Bg+Cg))×(Bbcn1−Ccb), and this value or a percentage thereof is added to all pixel values within said first in-focus portion and said second in-focus portion, and new color and intensity within in-focus portion of area Yn2=Yyx+(X/(X+Y))×(Yyx−Xxy) or more specifically Ccbn2=Ccb+((Ag+Bg)/(Ag+Bg+Cg))×(Ccb−Bbc), and this value or a percentage thereof is added to all pixel values within said third in-focus portion, where Xxy=Bbcn1, Yyx=Ccbn1, X=Ag+Bg, and Y=Cg are substituted into the generalized kernel equations to produce said Xn2=Bbcn2=Yn1=Ccbn2, and thus said second in-focus portion of said area Bg and said third in-focus portion of said area Cg are color and intensity corrected at said second-third focus locus edge to produce no discontinuity in color and intensity at said second-third focus locus edge, and this produces a new corresponding value of Bban2 which replaces Bban1 and Aabn2 which replaces Aabn1, and Aabn2 is equal to Bban2, the process of altering all pixels within said first in-focus portion and all pixels within said second in-focus portion and all pixels within said third in-focus portion constituting said simple three-component color and intensity equalization to produce a color and intensity equalized said composite photograph.
FF3. The software application of embodiment FF1 wherein said complex three-component composite photograph is formed using a complex three-component color and intensity equalization which comprises a first two-component color and intensity equalization followed by a second two-component color and intensity equalization followed by a third two-component color and intensity equalization followed by a fourth two-component color and intensity equalization, followed by at least one repetition of the above two-component color and intensity equalizations until there is no color and intensity discontinuity between any two of the component in-focus diagnostic photographs, said first in-focus portion of said area Ag of said complex three-component composite photograph has an average color and intensity value at said first-second focus locus edge of Aab, and said first in-focus portion of said area Ag of said complex three-component composite photograph has an average color and intensity value at said first-third focus locus edge of Aac, and said second in-focus portion of said area Bg of said complex three-component composite photograph has an average color and intensity value at said first-second focus locus edge of Bba and said second in-focus portion of said area Bg of said complex three-component composite photograph has an average color and intensity value at said second-third focus locus edge of Bbc, and said third in-focus portion of area C or more generally Cg of said complex three-component composite photograph has an average color and intensity value at said first-third focus locus edge of Cca and said third in-focus portion of said area Cg of said complex three-component composite photograph has an average color and intensity value at said second-third focus locus edge of Ccb, said first two-component color and intensity equalization employing the generalized kernel equations: new color and intensity within in-focus portion of area Xn1=Xxy+(Y/(X+Y))×(Xxy−Yyx), or more specifically Aabn1=Aab+(Bg/(Ag+Bg))×(Aab−Bba), and this value or a percentage thereof is added to all pixel values within said first in-focus portion, and new color and intensity within in-focus portion of area Yn1=Yyx+(X/(X+Y))×(Yyx−Xxy), or more specifically Bban1=Bba+(Ag/(Ag+Bg))×(Bba−Aab), and this value or a percentage thereof is added to all pixel values within said second in-focus portion, where Xxy=Aab, Yyx=Bba, X=Ag, and Y=Bg are substituted into the generalized kernel equations to produce said Xn1=Aabn1=Yn1=Bban1, and thus said first in-focus portion of said area Ag and said second in-focus portion of said area Bg are color and intensity corrected at said first-second focus locus edge to produce no discontinuity in color and intensity at said first-second focus locus edge, and this produces the new corresponding values of Aabn1, Aacn1, Bban1, Bbcn1, Ccan1, and Ccb1 where Aabn1 is equal to Bban1, Ccan1 is equal to Cca, and Ccbn1=Ccb; said second two-component color and intensity equalization employs the generalized kernel equations: new color and intensity within in-focus portion of area Xn2=Xxy+(Y/(X+Y))×(Xxy−Yyx), or more specifically ABacn2=Aacn1+(Cg/(Ag+Bg+Cg))×(Aacn1−Ccan1), and this value or a percentage thereof is added to all pixel values within said first in-focus portion and said second in-focus portion, and new color and intensity within in-focus portion of area Yn2=Yyx+(X/(X+Y))×(Yyx−Xxy) or more specifically Ccan2=Ccan1+((Ag+Bg)/(Ag+Bg+Cg))×(Ccan1−Aacn1), and this value or a percentage thereof is added to all pixel values within said third in-focus portion, where Xxy=Aacn1, Yyx=Ccan1, X=Ag+Bg, and Y=Cg are substituted into the generalized kernel equations to produce said Xn2=ABacn2=Yn2=Ccan2, and thus said first in-focus portion of said area Ag and said third in-focus portion of said area Cg are color and intensity corrected at said first-third focus locus edge to produce no discontinuity in color and intensity at said first-third focus locus edge, and this produces a new corresponding value of Bban2 which replaces Bban1 and Aabn2 which replaces Aabn1, and Aabn2 is equal to Bban2, and Bbcn2 replaces Bbcn1, and Ccbn2 replaces Ccbn1, said third two-component color and intensity equalization employs the generalized kernel equations: new color and intensity within in-focus portion of area Xn3=Xxy+(Y/(X+Y))×(Xxy−Yyx), or more specifically ACcbn3=Ccbn2+(Bg/(Ag+Bg+Cg))×(Ccbn2−Bbcn2), and this value or a percentage thereof is added to all pixel values within said first in-focus portion and said second in-focus portion, and new color and intensity within in-focus portion of area Yn3=Yyx+(X/(X+Y))×(Yyx−Xxy) or more specifically Bbcn3=Ccbn2+((Ag+Cg)/(Ag+Bg+Cg))×(Bbcn2−Ccbn2), and this value or a percentage thereof is added to all pixel values within said third in-focus portion, where Xxy=Ccbn2, Yyx=Bbcn2, X=Ag+Cg, and Y=Bg are substituted into the generalized kernel equations to produce said Xn3=ACcbn3=Yn3=Bbcn3, and thus said first in-focus portion of said area Ag and said third in-focus portion of said area Cg are color and intensity corrected at said second-third focus locus edge to produce no discontinuity in color and intensity at said first-third focus locus edge, and this produces a new corresponding value of Ccbn3 which replaces Ccbn2 and Bbcn3 which replaces Bbcn2 and Ccbn3 equals Bbcn3, and Aacn3 replaces Aacn2, and Ccan3 replaces Ccan2, and Aacn3 is equal to Ccan3, and Bban3 replaces Bban2 and Aabn3 replaces Aabn2, said fourth two-component color and intensity equalization employs the generalized kernel equations: new color and intensity within in-focus portion of area Xn4=Xxy+(Y/(X+Y))×(Xxy−Yyx), or more specifically BCbcn4=Bban3+(Ag/(Ag+Bg+Cg))×(Bban3−Aabn3), and this value or a percentage thereof is added to all pixel values within said second in-focus portion and said third in-focus portion, and new color and intensity within in-focus portion of area Yn4=Yyx+(X/(X+Y))×(Yyx−Xxy) or more specifically Aabn4=Aabn3+ ((Bg+Cg)/(Ag+Bg+Cg))×(Aabn3−Bban3), and this value or a percentage thereof is added to all pixel values within said first in-focus portion, where Xxy=Bban3, Yyx=Aabn3, X=Bg+Cg, and Y=Ag are substituted into the generalized kernel equations to produce said Xn4=BCbcn4=Yn4=Aabn4, and thus said first in-focus portion of said area Ag and said second in-focus portion of said area Bg are color and intensity corrected at said first-second focus locus edge to produce no discontinuity in color and intensity at said first-second focus locus edge, and this produces a new corresponding value of Ccbn4 which replaces Ccbn3 and Bbcn4 which replaces Bbcn3 and Ccbn4 equals Bbcn4, and Aacn4 replaces Aacn3, and Ccan4 replaces Ccan3, and Bban4 replaces Bban3 and Aabn4 replaces Aabn3 and Bban4 is equal to Aabn4, said second two-component color and intensity equalization followed by said third two-component color and intensity equalization followed by said fourth two-component color and intensity equalization repeats as many times as is necessary until there is no discontinuity in color and intensity at said first-second focus locus edge and at said first-third focus locus edge and at said second-third focus locus edge, the process of altering all pixels within said first in-focus portion and all pixels within said second in-focus portion and all pixels within said third in-focus portion constituting said complex three-component color and intensity equalization to produce a color and intensity equalized said composite photograph.
FF4. The software application of embodiment FF further comprising: the creation of at least one parametric coordinate system comprising a multiplicity of parametric pixels that superimpose upon the rectangular pixel grid comprising a multiplicity of rectangular pixels taken by a digital camera, and the intensity and color value of each parametric pixel being contained within the set of said multiplicity of parametric pixels is determined by the weighted average of the intensity and color values a subset of said multiplicity of overlapped portions of rectangular pixels intersecting said parametric pixel; at least one mathematical operation performed on and between at least one first subset of said multiplicity of parametric pixels and at least one second subset of said multiplicity of parametric pixels, the redefining of the intensity and color value of at least one transformed rectangular pixel overlapped by a multiplicity of parametric pixels that superimpose upon said transformed rectangular pixel, and the intensity and color value of each transformed rectangular pixel is determined by the weighted average of the intensity and color values of a subset of said multiplicity of overlapped portions of parametric pixels intersecting said rectangular pixel.
GG. The software application of embodiment AA1 wherein said angular alignment guidance subsystem comprises or further comprises the ability to acquire the reflection of a smartphone within the eye to determine the horizontal component of displacement along a horizontal axis and the vertical component of displacement along a vertical axis between the eye central axis and said display central axis, and said horizontal component and said vertical component is used in whole or supplementally in part to create at least one feedback sound to position said smartphone by a user.
GG1. The software application of embodiment AA1 further comprising the ability to recognize the reflection of a cellphone, smartphone, person, or other recognizable objects and subtract these features or a portion thereof from a photograph or composite photograph of an eye.
KK. The software application of embodiment AA1 wherein said at least one diagnostic photograph or at least one composite diagnostic photograph is illumination and camera color-corrected to produce at least one illumination and camera color-corrected diagnostic photograph or at least one illumination and camera color-corrected composite diagnostic photograph by employing at least one illumination and camera color-correction transform or transfer function that maps every color and intensity of each pixel of said diagnostic photograph or composite diagnostic photograph into a different color and intensity of each respective pixel to produce said illumination and camera color-corrected diagnostic photograph or illumination and camera color-corrected composite diagnostic photograph.
KK1. The software application of embodiment KK wherein said at least one diagnostic photograph or at least one composite diagnostic photograph or at least one illumination and camera color-corrected diagnostic photograph or at least one illumination and camera color-corrected composite diagnostic photograph is condition-specific color-altered by employing at least one condition-specific color-correction transform or transfer function that maps every color and intensity of each pixel of said diagnostic photograph or said composite diagnostic photograph or said illumination and camera color-corrected diagnostic photograph or said illumination and camera color-corrected composite diagnostic photograph into a different color and intensity of each respective pixel to produce at least one condition-specific color-corrected diagnostic photograph or at least one condition-specific color-corrected composite diagnostic photograph or at least one condition-specific color-corrected illumination and camera color-corrected diagnostic photograph or at least one condition-specific color-corrected illumination and camera color-corrected composite diagnostic photograph.
DD. The software application of embodiment AA1 wherein said eye or other body part is anything that can be photographed.
LL. The software application of embodiment AA1 further comprising a master crop function to reduce the size of said diagnostic photograph or said composite diagnostic photograph to produce a normalized diagnostic photograph or normalized composite diagnostic photograph, said master crop function producing at least one of the following shapes: rectangular, circular, elliptical, or any bounding shape.
MM. The software application of embodiment KK1 further comprising at least one transform applied to said at least one diagnostic photograph or at least one composite diagnostic photograph or at least one illumination and camera color-corrected diagnostic photograph or at least one illumination and camera color-corrected composite diagnostic photograph or at least one condition-specific color-corrected diagnostic photograph or at least one condition-specific color-corrected composite diagnostic photograph or at least one condition-specific color-corrected illumination and camera color-corrected diagnostic photograph or at least one condition-specific color-corrected illumination and camera color-corrected composite diagnostic photograph for use in analysis and diagnostics, said at least one transform is or employs at least one of: an FFT, a discrete Fourier transform, Walsh transform, Hadamard transform, cosine transform, discrete wavelet transform, Laplace transform, convolution integrals and convolution processing, Canny edge detection, Prewitt algorithm for edge detection, Sobel operators for edge detection, or any transform now known or unknown that can be applied to or act upon a two-dimensional array or image or three-dimensional array or n-dimensional array to increase diagnostic discrimination between possessing and not possessing a particular medical condition or classification or impairment.
RR. An application that transforms a smartphone camera into a change detection alarm comprising: setting up smartphone so camera is watching the subject field; initiating video mode; analyzing in real time at least one video frame; detecting at least one change of at least one attribute in at least one location within said at least one video frame; establishing in advance a user-selectable threshold of at least one change of at least one attribute in at least one location; generating at least one alarm when said threshold of at least one change has been reached or exceeded.
NN1. A software application that transforms a smartphone containing at least one camera and a visual display with a display central axis into a user impairment diagnostic system comprising the ability to: establish a user baseline response comprising: taking at least one video or at least one photograph of a user; aligning the smartphone so said user is centered about said display central axis; generating at least one stimulus target image or stimulus sound or stimulus target image and stimulus sound, said stimulus target image to be displayed on said visual display, said stimulus sound to be produced by the smartphone speaker; measuring the user response to said stimulus that is at least one of: the user response to visually follow said at least one stimulus target image, user response to keep an image in focus, measure and assess motor coordination based on user image parameter response, determine motor coordination based on acceleration data acquired by at least one accelerometer or at least one gyroscope or at least one accelerometer and gyroscope or at least one sensor available on a said smartphone, measure user pupil dilation to at least one brightness change, measure and quantify user facial expression parameters, measure and quantify user gait, measure, quantify, and assess other biometric parameters that can be sensed by a smartphone, the measurement of user response to at least one said stimulus sound in a prescribed manner, said user response comprising at least one of: head movement, at least one eye movement, pupil dilation, at least one hand movement, at least one finger movement, walking, standing, turning, or any combination thereof; generate at least one metric to establish user baseline performance; establish at least one user test response comprising: taking a at least one video or at least one photograph of a user; aligning the smartphone so said user is centered about said display central axis; generating at least one stimulus target image or stimulus sound or stimulus target image and stimulus sound, said stimulus target image to be displayed on said visual display, said stimulus sound to be produced by the smartphone speaker; measuring the user response to said stimulus that is at least one of: the user response to visually follow said at least one stimulus target image, user response to keep an image in focus, measure and assess motor coordination based on user image parameter response, determine motor coordination based on acceleration data acquired by at least one accelerometer or at least one gyroscope or at least one accelerometer and gyroscope or at least one sensor available on a said smartphone, measure user pupil dilation to at least one brightness change, measure and quantify user facial expression parameters, measure and quantify user gait, measure, quantify, and assess other biometric parameters that can be sensed by a cellphone, the measurement of user response to at least one said stimulus sound in a prescribed manner, said user response comprising at least one of: head movement, at least one eye movement, pupil dilation, at least one hand movement, at least one finger movement, walking, standing, turning, or any combination thereof; generate at least one metric to establish user test performance; compare said at least one metric of said user test performance with said at least one metric of said user baseline performance to establish the probability of user impairment, and associated confidence level of user impairment assessment; store results in at least one database located on said smartphone or upload said results to at least one remote database located in at least one remote location or store results in at least one database located on said smartphone and upload said results to at least one remote database located in at least one remote location; send at least one notification.
PP. A portable display device that generates at least two visible outputs comprising: a microprocessor; at least one power source; a power management strategy to power down when not in use for at least one period of time to conserve power; a shell or plate or backplane or housing; a hook or hole from which said portable display device can be hung against a wall or vertical surface and a fold-out stand to enable standing on a horizontal surface; at least two arms that can extend from said shell or plate or backplane or housing; said at least two arms are either fixed or hinged arms or arms attached by pivots that can rotate or splay radially outward or are telescoping arms that can extend radially outward or a combination of both; said shell or plate or backplane or housing contains detents that secure or affix at at least two angles said fixed or hinged arms or arms attached by pivots or said telescoping arms; said fixed or hinged arms or arms attached by pivots or said telescoping arms each contain at least one light source distally located that can be switched on or off by said microprocessor; a receiving subsystem that is at least one of wireless, wired, acoustically communicated with; said receiving subsystem receiving signals and commands from at least one smartphone and communicating with or is controlled by said microprocessor.
SS. A method of transforming a smart device into a medical diagnostic tool comprising the following steps: downloading an application from at least one source; positioning with the aid of audio feedback a smart device to take at least one photograph containing at least one in-focus portion of a target image during a training session; positioning with the aid of audio feedback a smart device to take at least one photograph containing at least one in-focus portion of a target image to produce at least one diagnostic photograph; creating at least one in-focus composite diagnostic photograph from the in-focus portions of said at least one photograph; illumination and camera color correcting said at least one composite diagnostic photograph; condition-specific color correcting said at least one composite diagnostic photograph or illumination and camera color corrected composite diagnostic photograph; cropping said composite diagnostic photograph; applying at least one other transform to increase diagnostic signal to noise discrimination; comparing said diagnostic composite photograph to at least one database to determine the probability of possessing or not possessing at least one condition or level of substance impairment herein referred to as the result; encrypting or decrypting or encrypting and decrypting before or after any of the above operations depending on where a modification operation is being performed or stored; storing the result to at least one database; sending at least one notification.
1. A computer-implemented method for capturing a diagnostic image of a target using a computing device having at least one camera, at least one processor, and at least one audio output, the method comprising the steps of: initiating a video stream from the at least one camera to establish a real-time display of the target within a display area of the computing device, the display area having a display central axis; determining, from at least one image frame of the video stream, a horizontal displacement and a vertical displacement of the target relative to the display central axis, and an angular orientation of the target relative to the at least one camera; generating, via the at least one audio output, at least one angular alignment feedback sound having at least one audio parameter that varies proportionally to the horizontal displacement, the vertical displacement, and the angular orientation, to guide a user to position the computing device so that the target is centered about the display central axis and the camera is oriented normal to the target; ceasing the at least one angular alignment feedback sound upon achieving angular alignment, wherein angular alignment is the condition in which the target is centered about the display central axis and the camera is normal to the target; generating, upon achieving angular alignment, a focal distance feedback sound having a focal audio parameter that varies proportionally to the absolute value of the difference between a current distance from the camera to the target and a predetermined optimal focal distance, the focal audio parameter reaching a peak condition when the camera is at the optimal focal distance; capturing at least one diagnostic photograph upon achieving the peak condition; evaluating the at least one diagnostic photograph to determine at least one in-focus portion and at least one out-of-focus portion thereof; when the at least one diagnostic photograph is only partially in focus, capturing at least one additional diagnostic photograph and computing a composite diagnostic photograph from only the in-focus portions of the at least one diagnostic photograph and the at least one additional diagnostic photograph; and applying at least one image processing transform to the composite diagnostic photograph to produce a processed diagnostic image suitable for machine learning-based diagnostic analysis.
2. The method according to embodiment 1, further comprising the step of implementing a user application residing in memory on the computing device.
3. The method according to embodiments 1-2, wherein the at least one angular alignment feedback sound comprises a first audio sound type and a second audio sound type generated simultaneously, the first audio sound type being a continuously varying fundamental frequency proportional to the absolute value of the horizontal displacement and rotation about the vertical axis, and the second audio sound type being a pulse train having a pulse repetition rate proportional to the absolute value of the vertical displacement and rotation about the horizontal axis.
4. The method according to embodiments 1-3, wherein the at least one image processing transform comprises an illumination and camera color-correction transform followed by a condition-specific color-conditioning transform, the illumination and camera color-correction transform mapping the color and intensity values of each pixel of the composite diagnostic photograph to normalized values compensating for camera filter spectral response and illumination source spectrum, and the condition-specific color-conditioning transform further mapping the normalized values to conditioned values selected to maximize discrimination between images possessing and not possessing a specific medical condition or level of substance impairment.
5. The method according to embodiments 1-4, further comprising the steps of: comparing the processed diagnostic image against a first labeled dataset comprising images representative of a condition of interest and against a second labeled dataset comprising images not representative of the condition of interest using a machine learning classifier; generating a classification result comprising a determination of whether the processed diagnostic image belongs to the first labeled dataset; and generating a confidence level associated with the classification result.
6. The method according to embodiments 1-5, further comprising the steps of: encrypting the processed diagnostic image and the classification result prior to transmission; transmitting the encrypted processed diagnostic image and classification result to at least one remote location; and storing the processed diagnostic image, the classification result, and the confidence level in at least one database.
7. The method according to embodiments 1-6, wherein the step of encrypting the processed diagnostic image further comprises the steps of: generating a per-image symmetric encryption key; encrypting the processed diagnostic image using the per-image symmetric encryption key; and encrypting the per-image symmetric encryption key using a public key of at least one authorized recipient.
8. The method according to embodiments 1-7, further comprising the steps of: computing a cryptographic hash of the processed diagnostic image and associated metadata at the time of image creation; storing the cryptographic hash in a local encrypted database on the computing device; and verifying, upon retrieval of the processed diagnostic image, that a recomputed cryptographic hash matches the stored cryptographic hash.
9. The method according to embodiments 1-8, further comprising the steps of: establishing a mutual authentication session between the computing device and at least one remote server using a forward-secrecy key exchange protocol; and transmitting the encrypted processed diagnostic image within the mutual authentication session together with a digital signature generated using a private key of the user.
10. The method according to embodiments 1-9, wherein the machine learning classifier is executed on the computing device that captured the at least one diagnostic photograph using an on-device inference framework.
11. The method according to embodiments 1-10, wherein the machine learning classifier is executed on at least one remote server, the method further comprising the steps of: encrypting the processed diagnostic image prior to transmission to the at least one remote server; decrypting the encrypted processed diagnostic image at the at least one remote server; executing the machine learning classifier on the decrypted processed diagnostic image; encrypting the classification result and confidence level; and transmitting the encrypted classification result and confidence level to the computing device.
12. The method according to embodiments 1-11, wherein: the machine learning classifier is a multi-class classifier trained to simultaneously classify the processed diagnostic image into one of a plurality of condition classes; and the classification result comprises a vector of per-class confidence levels.
13. The method according to embodiments 1-12, further comprising the steps of: displaying a stimulus target on the computing device display; capturing a video of a user's eye response to the stimulus target; determining a saccadic latency by measuring a time elapsed between onset of the stimulus target and initiation of eye movement toward the stimulus target; comparing the saccadic latency to a baseline saccadic latency established for the user; and generating an impairment indicator when the saccadic latency deviates from the baseline saccadic latency by more than a threshold amount.
14. The method according to embodiments 1-13, further comprising the steps of: displaying a second stimulus target moving continuously across the computing device display; capturing a video of the user's eye tracking the second stimulus target; computing a smooth pursuit gain coefficient as a ratio of measured eye velocity to second stimulus target velocity; detecting saccadic intrusions in the tracking motion; and incorporating the smooth pursuit gain coefficient and a frequency of saccadic intrusions into the impairment indicator.
15. The method according to embodiments 1-14, further comprising the steps of: presenting a light stimulus of a controlled intensity and duration via the computing device display; capturing a video of a user's pupil response to the light stimulus; extracting at least one pupillary light reflex metric comprising at least one of pupil constriction latency, maximum constriction amplitude, constriction velocity, or re-dilation velocity; and incorporating the at least one pupillary light reflex metric into the impairment indicator.
16. The method according to embodiments 1-15, further comprising the steps of: acquiring accelerometer and gyroscope data from the computing device while the user performs at least one guided motor task; computing at least one motor coordination metric comprising at least one of postural tremor frequency, postural tremor amplitude, finger trajectory smoothness, or reaction time; and incorporating the at least one motor coordination metric into the impairment indicator.
17. The method according to embodiments 1-16, wherein the impairment indicator comprises a probability of impairment and an identification of at least one probable impairment category selected from alcohol impairment, cannabis impairment, stimulant impairment, opioid impairment, sedative impairment, fatigue impairment, neurological impairment, and no impairment detected.
18. The method according to embodiments 1-17, wherein: the target is a skin lesion; the at least one image processing transform comprises a dermatological color-conditioning transform configured to enhance contrast between melanin distribution patterns and surrounding skin tissue; and the method further comprises the step of classifying the processed diagnostic image using a machine learning classifier trained on labeled skin lesion images.
19. The method according to embodiments 1-18, wherein: the target is an interior surface of an oral cavity; the at least one image processing transform comprises an oral mucosal color-conditioning transform; and the method further comprises the step of classifying the processed diagnostic image using a machine learning classifier trained on labeled oral cavity images.
20. The method according to embodiments 1-19, wherein: the target is a wound bed; and the method further comprises the steps of: capturing serial diagnostic photographs of the wound bed at a plurality of time points; computing a change in at least one wound characteristic over time comprising at least one of: wound area, wound depth, tissue composition, or presence of infection indicators; and transmitting the computed change to a remote healthcare provider.
21. The method according to embodiments 1-20, wherein the at least one audio parameter of the angular alignment feedback sound varies as a function of both a current position sample and at least one previous position sample to produce a corrective sound that anticipates the user's motion trajectory and reduces positioning time.
22. A diagnostic imaging system comprising: a computing device comprising at least one camera, at least one display, at least one processor, at least one audio output device, and at least one non-transitory computer-readable medium storing instructions that, when executed by the at least one processor, cause the computing device to: determine a horizontal displacement, a vertical displacement, and an angular orientation of a target relative to a central axis of the at least one display; generate a first audio sound type having a first audio parameter proportional to the horizontal displacement and rotation about a vertical axis of the computing device; generate a second audio sound type having a second audio parameter proportional to the vertical displacement and rotation about a horizontal axis of the computing device; cease the first audio sound type and the second audio sound type upon achieving angular alignment of the computing device with the target; generate a focal audio sound type having a focal audio parameter proportional to a distance between the at least one camera and the target, the focal audio parameter reaching a peak condition at a predetermined optimal focal distance; capture at least one diagnostic photograph of the target upon achieving the peak condition; construct a composite diagnostic photograph from in-focus portions of the at least one diagnostic photograph; apply an illumination and camera color-correction transform to the composite diagnostic photograph; and apply a condition-specific color-conditioning transform to the color-corrected composite diagnostic photograph to produce a conditioned diagnostic image.
23. The system according to embodiment 22, wherein: the target is a human eye; and the at least one processor is further configured to determine the angular orientation by: detecting a boundary of the iris or pupil of the eye; fitting a best-fit ellipse to the detected boundary; computing an ellipse major axis, an ellipse minor axis, and an angle between the ellipse minor axis and a horizontal axis of the display; and using the angle to compute a component of the first audio parameter and a component of the second audio parameter.
24. The system according to embodiments 22-23, wherein the condition-specific color-conditioning transform is selected from a plurality of condition-specific color-conditioning transforms each associated with a respective medical condition or substance, the plurality of medical conditions or substances comprising at least two of: malaria, diabetic retinopathy, COVID-19, conjunctivitis, alcohol impairment, cannabis impairment, opiate impairment, and amphetamine impairment.
25. The system according to embodiments 22-24, wherein the instructions further cause the computing device to: detect at least one specular reflection within a corneal region of a diagnostic photograph of a human eye; segment the at least one specular reflection from surrounding iris and scleral tissue; and replace the segmented region with synthesized image content interpolated from surrounding non-reflected tissue to produce a reflection-corrected diagnostic photograph.
26. The system according to embodiments 22-25, wherein detecting the at least one specular reflection comprises applying a trained object recognition model to identify a computing device reflection having a characteristic rectangular shape within the corneal region.
27. The system according to embodiments 22-26, wherein the instructions further cause the computing device to: determine, from a position of a corneal reflex within the corneal region of the eye, a component of the angular misalignment between the camera and the optical axis of the eye; and incorporate the component of the angular misalignment derived from the corneal reflex position into the at least one angular alignment feedback sound.
28. The system according to embodiments 22-27, further comprising a portable display device in wireless communication with the computing device, the portable display device comprising at least two arms extending from a housing, each arm having at least one light source at a distal end that is individually controllable by a microprocessor of the portable display device in response to commands received from the computing device, the at least two arms being configured to position the at least one light source at an angular displacement from a central axis of the portable display device sufficient to test for horizontal gaze nystagmus.
29. The system according to embodiments 22-28, wherein: the portable display device further comprises at least one orientation sensor selected from the group consisting of an accelerometer, a gyroscope, and a magnetometer; and the computing device receives orientation data from the at least one orientation sensor via the wireless communication link and uses the orientation data to compute the precise angular position of each light source relative to the user's line of sight.
30. The system according to embodiments 22-29, wherein: each light source of the portable display device is an LED or LED array capable of emitting light at a controllable intensity and in at least one selectable color; and the microprocessor of the portable display device executes light source switching commands received from the computing device with a response latency of less than 50 milliseconds.
31. The system according to embodiments 22-30, wherein: the computing device measures a round-trip communication latency between transmitting a light source command and confirming execution of the command; and the computing device subtracts the round-trip communication latency from a measured user response time to yield a net physiological response latency attributable to the user.
32. A computer-implemented method for constructing a fully in-focus composite image from a plurality of partially in-focus photographs, the method comprising the steps of: receiving a first photograph and determining a first in-focus portion and a first out-of-focus portion thereof, the first in-focus portion being bounded by a first focus locus boundary; receiving a second photograph and determining a second in-focus portion and a second out-of-focus portion thereof, the second in-focus portion being bounded by a second focus locus boundary; computing a tiling focus intersection boundary between the first focus locus boundary and the second focus locus boundary; cropping the first photograph at the tiling focus intersection boundary to produce a first cropped in-focus portion and cropping the second photograph at the tiling focus intersection boundary to produce a second cropped in-focus portion; logically combining the first cropped in-focus portion and the second cropped in-focus portion to form a composite image in which the entire composite image is in focus and no pixels are blank or overlapped; determining an average color and intensity value of pixels of the first cropped in-focus portion at the tiling focus intersection boundary and an average color and intensity value of pixels of the second cropped in-focus portion at the tiling focus intersection boundary; and applying a weighted color and intensity equalization to all pixels of the first cropped in-focus portion and all pixels of the second cropped in-focus portion to eliminate color and intensity discontinuity at the tiling focus intersection boundary.
33. The method according to embodiment 32, wherein the step of computing the tiling focus intersection boundary further comprises the step of determining a midpoint boundary between the first focus locus boundary and the second focus locus boundary such that the tiling focus intersection boundary lies entirely within the in-focus region of both the first photograph and the second photograph.

In closing, regarding the exemplary embodiments of the present invention as shown and described herein, it will be appreciated that a system and associated methods are disclosed and configured for capturing and processing images of a target body part for diagnostic purposes. Because the principles of the invention may be practiced in a number of configurations beyond those shown and described, it is to be understood that the invention is not in any way limited by the exemplary embodiments, but is generally directed to an image capture and processing system and is able to take numerous forms to do so without departing from the spirit and scope of the invention.

Certain embodiments of the present invention are described herein, including the best mode known to the inventor(s) for carrying out the invention. Of course, variations on these described embodiments will become apparent to those of ordinary skill in the art upon reading the foregoing description. The inventor(s) expect skilled artisans to employ such variations as appropriate, and the inventor(s) intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the terms “about” and “approximately.” As used herein, the terms “about” and “approximately” mean that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein. Similarly, as used herein, unless indicated to the contrary, the term “substantially” is a term of degree intended to indicate an approximation of the characteristic, item, quantity, parameter, property, or term so qualified, encompassing a range that can be understood and construed by those of ordinary skill in the art, or at least encompassing a range of plus or minus ten percent above and below the value of the stated characteristic, item, quantity, parameter, property, or term.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators-such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (along with equivalent open-ended transitional phrases thereof such as “including,” “containing” and “having”) encompasses all the expressly recited elements, limitations, steps and/or features alone or in combination with un-recited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amendment for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim, whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (along with equivalent open-ended transitional phrases thereof) includes within its meaning, as a limiting case, claimed subject matter specified by the closed-ended transitional phrases “consisting of” or “consisting essentially of.” As such, embodiments described herein or so claimed with the phrase “comprising” are expressly or inherently unambiguously described, enabled and supported herein for the phrases “consisting essentially of” and “consisting of.”

Any claims intended to be treated under 35 U.S.C. § 112 (f) will begin with the words “means for,” but use of the term “for” in any other context is not intended to invoke treatment under 35 U.S.C. § 112 (f). Accordingly, Applicant reserves the right to pursue additional claims after filing this application, in either this application or in a continuing application.

It should be understood that any logic code, programs, modules, processes, and/or methods disclosed herein, along with the order in which the respective elements of any such method are performed, are purely exemplary. Depending on the implementation, they may be performed in any order or in parallel, unless indicated otherwise in the present disclosure. Further, the logic code is not related, or limited to any particular programming language, and may comprise one or more modules that execute on one or more processors in a distributed, non-distributed, or multiprocessing environment. Additionally, the various illustrative logical blocks, modules, methods, and algorithm processes and sequences described in connection with the embodiments disclosed herein can be implemented as electronic hardware, computer software, or combinations of both. To clearly illustrate this interchangeability of hardware and software, various illustrative components, blocks, modules, and process actions have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of this document.

The phrase “non-transitory,” in addition to having its ordinary meaning, as used in this document means “enduring or long-lived”. The phrase “non-transitory computer readable medium,” in addition to having its ordinary meaning, includes any and all computer readable mediums, with the sole exception of a transitory, propagating signal. This includes, by way of example and not limitation, non-transitory computer-readable mediums such as register memory, processor cache and random-access memory (“RAM”).

The methods as described above may be used in the fabrication of integrated circuit chips. The resulting integrated circuit chips can be distributed by the fabricator in raw wafer form (that is, as a single wafer that has multiple unpackaged chips), as a bare die, or in a packaged form. In the latter case, the chip is mounted in a single chip package (such as a plastic carrier, with leads that are affixed to a motherboard or other higher level carrier) or in a multi-chip package (such as a ceramic carrier that has either or both surface interconnections or buried interconnections). In any case, the chip is then integrated with other chips, discrete circuit elements, and/or other signal processing devices as part of either (a) an intermediate product, such as a motherboard, or (b) an end product. The end product can be any product that includes integrated circuit chips, ranging from toys and other low-end applications to advanced computer products having a display, a keyboard or other input device, and a central processor.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and does not constitute any admission as to the correctness of the dates or contents of these documents.

While aspects of the invention have been described with reference to at least one exemplary embodiment, it is to be clearly understood by those skilled in the art that the invention is not limited thereto. Rather, the scope of the invention is to be interpreted only in conjunction with the appended claims and it is made clear, here, that the inventor(s) believe that the claimed subject matter is the invention.

All of the material in this patent document issue subject to copyright protection under the copyright laws of the United States and other countries. The copyright owner has no objection to the facsimile reproduction by anyone of the patent document or the patent disclosure, as it appears in official governmental records but, otherwise, all other copyright rights whatsoever are reserved.

1. A computer-implemented method for capturing a diagnostic image of a target using a computing device having at least one camera, at least one processor, and at least one audio output, the method comprising the steps of:
initiating a video stream from the at least one camera to establish a real-time display of the target within a display area of the computing device, the display area having a display central axis;
determining, from at least one image frame of the video stream, a horizontal displacement and a vertical displacement of the target relative to the display central axis, and an angular orientation of the target relative to the at least one camera;
generating, via the at least one audio output, at least one angular alignment feedback sound having at least one audio parameter that varies proportionally to the horizontal displacement, the vertical displacement, and the angular orientation, to guide a user to position the computing device so that the target is centered about the display central axis and the camera is oriented normal to the target;
ceasing the at least one angular alignment feedback sound upon achieving angular alignment, wherein angular alignment is the condition in which the target is centered about the display central axis and the camera is normal to the target;
generating, upon achieving angular alignment, a focal distance feedback sound having a focal audio parameter that varies proportionally to the absolute value of the difference between a current distance from the camera to the target and a predetermined optimal focal distance from the camera to the target;
capturing at least one diagnostic photograph upon the camera reaching the optimal focal distance from the camera to the target;
evaluating the at least one diagnostic photograph to determine at least one in-focus portion and at least one out-of-focus portion thereof;
when the at least one diagnostic photograph is only partially in focus, capturing at least one additional diagnostic photograph and computing a composite diagnostic photograph from only the in-focus portions of the at least one diagnostic photograph and the at least one additional diagnostic photograph; and
applying at least one image processing transform to the composite diagnostic photograph to produce a processed diagnostic image suitable for machine learning-based diagnostic analysis.

2. The method of claim 1, wherein the at least one angular alignment feedback sound comprises a first audio sound type and a second audio sound type generated simultaneously, the first audio sound type being a continuously varying fundamental frequency proportional to the absolute value of the horizontal displacement and rotation about the vertical axis, and the second audio sound type being a pulse train having a pulse repetition rate proportional to the absolute value of the vertical displacement and rotation about the horizontal axis.

3. The method of claim 1, wherein the at least one image processing transform comprises an illumination and camera color-correction transform followed by a condition-specific color-conditioning transform, the illumination and camera color-correction transform mapping the color and intensity values of each pixel of the composite diagnostic photograph to normalized values compensating for camera filter spectral response and illumination source spectrum, and the condition-specific color-conditioning transform further mapping the normalized values to conditioned values selected to maximize discrimination between images possessing and not possessing a specific medical condition or level of substance impairment.

4. The method of claim 1, further comprising the steps of:
comparing the processed diagnostic image against a first labeled dataset comprising images representative of a condition of interest and against a second labeled dataset comprising images not representative of the condition of interest using a machine learning classifier;
generating a classification result comprising a determination of whether the processed diagnostic image belongs to the first labeled dataset; and
generating a confidence level associated with the classification result.

5. The method of claim 4, wherein:
the machine learning classifier is a multi-class classifier trained to simultaneously classify the processed diagnostic image into one of a plurality of condition classes; and
the classification result comprises a vector of per-class confidence levels.

6. The method of claim 1, further comprising the steps of:
displaying a stimulus target on the computing device display;
capturing a video of a user's eye response to the stimulus target;
determining a saccadic latency by measuring a time elapsed between onset of the stimulus target and initiation of eye movement toward the stimulus target;
comparing the saccadic latency to a baseline saccadic latency established for the user; and
generating an impairment indicator when the saccadic latency deviates from the baseline saccadic latency by more than a threshold amount.

7. The method of claim 6, further comprising the steps of:
displaying a second stimulus target moving continuously across the computing device display;
capturing a video of the user's eye tracking the second stimulus target;
computing a smooth pursuit gain coefficient as a ratio of measured eye velocity to second stimulus target velocity;
detecting saccadic intrusions in the tracking motion; and
incorporating the smooth pursuit gain coefficient and a frequency of saccadic intrusions into the impairment indicator.

8. The method of claim 6, further comprising the steps of:
presenting a light stimulus of a controlled intensity and duration via the computing device display;
capturing a video of a user's pupil response to the light stimulus;
extracting at least one pupillary light reflex metric comprising at least one of pupil constriction latency, maximum constriction amplitude, constriction velocity, or re-dilation velocity; and
incorporating the at least one pupillary light reflex metric into the impairment indicator.

9. The method of claim 6, further comprising the steps of:
acquiring accelerometer and gyroscope data from the computing device while the user performs at least one guided motor task;
computing at least one motor coordination metric comprising at least one of postural tremor frequency, postural tremor amplitude, finger trajectory smoothness, or reaction time; and
incorporating the at least one motor coordination metric into the impairment indicator.

10. A diagnostic imaging system comprising:
a computing device comprising at least one camera, at least one display, at least one processor, at least one audio output device, and at least one non-transitory computer-readable medium storing instructions that, when executed by the at least one processor, cause the computing device to:
determine a horizontal displacement, a vertical displacement, and an angular orientation of a target relative to a central axis of the at least one display;
generate a first audio sound type having a first audio parameter proportional to the horizontal displacement and rotation about a vertical axis of the computing device;
generate a second audio sound type having a second audio parameter proportional to the vertical displacement and rotation about a horizontal axis of the computing device;
cease the first audio sound type and the second audio sound type upon achieving angular alignment of the computing device with the target;
generate a focal audio sound type having a focal audio parameter proportional to a distance between the at least one camera and the target;
capture at least one diagnostic photograph of the target upon the camera reaching a predetermined optimal focal distance from the camera to the target;
construct a composite diagnostic photograph from in-focus portions of the at least one diagnostic photograph;
apply an illumination and camera color-correction transform to the composite diagnostic photograph; and
apply a condition-specific color-conditioning transform to the color-corrected composite diagnostic photograph to produce a conditioned diagnostic image.

11. The system of claim 10, wherein:
the target is a human eye; and
the at least one processor is further configured to determine the angular orientation by:
detecting a boundary of the iris or pupil of the eye;
fitting a best-fit ellipse to the detected boundary;
computing an ellipse major axis, an ellipse minor axis, and an angle between the ellipse minor axis and a horizontal axis of the display; and
using the angle to compute a component of the first audio parameter and a component of the second audio parameter.

12. The system of claim 10, wherein the instructions further cause the computing device to:
detect at least one specular reflection within a corneal region of a diagnostic photograph of a human eye;
segment the at least one specular reflection from surrounding iris and scleral tissue; and
replace the segmented region with synthesized image content interpolated from surrounding non-reflected tissue to produce a reflection-corrected diagnostic photograph.

13. The system of claim 12, wherein detecting the at least one specular reflection comprises applying a trained object recognition model to identify a computing device reflection having a characteristic rectangular shape within the corneal region.

14. The system of claim 10, wherein the instructions further cause the computing device to:
determine, from a position of a corneal reflex within the corneal region of the eye, a component of the angular misalignment between the camera and the optical axis of the eye; and
incorporate the component of the angular misalignment derived from the corneal reflex position into the at least one angular alignment feedback sound.

15. The system of claim 10, further comprising a portable display device in wireless communication with the computing device, the portable display device comprising at least two arms extending from a housing, each arm having at least one light source at a distal end that is individually controllable by a microprocessor of the portable display device in response to commands received from the computing device, the at least two arms being configured to position the at least one light source at an angular displacement from a central axis of the portable display device sufficient to test for horizontal gaze nystagmus.

16. The system of claim 15, wherein:
the portable display device further comprises at least one orientation sensor selected from the group consisting of an accelerometer, a gyroscope, and a magnetometer; and
the computing device receives orientation data from the at least one orientation sensor via the wireless communication link and uses the orientation data to compute the precise angular position of each light source relative to the user's line of sight.

17. The system of claim 15, wherein:
each light source of the portable display device is an LED or LED array capable of emitting light at a controllable intensity and in at least one selectable color; and
the microprocessor of the portable display device executes light source switching commands received from the computing device with a response latency of less than 50 milliseconds.

18. The system of claim 15, wherein:
the computing device measures a round-trip communication latency between transmitting a light source command and confirming execution of the command; and
the computing device subtracts the round-trip communication latency from a measured user response time to yield a net physiological response latency attributable to the user.

19. A computer-implemented method for constructing a fully in-focus composite image from a plurality of partially in-focus photographs, the method comprising the steps of:
receiving a first photograph and determining a first in-focus portion and a first out-of-focus portion thereof, the first in-focus portion being bounded by a first focus locus boundary;
receiving a second photograph and determining a second in-focus portion and a second out-of-focus portion thereof, the second in-focus portion being bounded by a second focus locus boundary;
computing a tiling focus intersection boundary between the first focus locus boundary and the second focus locus boundary;
cropping the first photograph at the tiling focus intersection boundary to produce a first cropped in-focus portion and cropping the second photograph at the tiling focus intersection boundary to produce a second cropped in-focus portion;
logically combining the first cropped in-focus portion and the second cropped in-focus portion to form a composite image in which the entire composite image is in focus and no pixels are blank or overlapped;
determining an average color and intensity value of pixels of the first cropped in-focus portion at the tiling focus intersection boundary and an average color and intensity value of pixels of the second cropped in-focus portion at the tiling focus intersection boundary; and
applying a weighted color and intensity equalization to all pixels of the first cropped in-focus portion and all pixels of the second cropped in-focus portion to eliminate color and intensity discontinuity at the tiling focus intersection boundary.

20. The method of claim 19, wherein the step of computing the tiling focus intersection boundary further comprises the step of determining a midpoint boundary between the first focus locus boundary and the second focus locus boundary such that the tiling focus intersection boundary lies entirely within the in-focus region of both the first photograph and the second photograph.
